SEQUENCE LISTING (24 sequences)

SEQ ID NO: 1 - DNA, 604 bp, Artificial sequence
  Synthetic construct
atggcacatcaccaccaccatcacgtgggtaccggcagcggcaagggcaagggcagcggcagcggtttttggcgtcacaacagcggtcgtaacccggaggagaaagacaacttcctgagccgtaacaaggagaaccacaaaccgaccgagagcagcctggacgaaaaggttgcgccgagcaaagcgagccagaccaccggtggcttcagccaaccgtgcctgccgaactggatcatgcacggcaagagctgctacctgttcagctttagcggtaacagctggtatggcagcaaacgtcattgcagccagctgggtgcgcacctgctgaagatcgacaacagcaaagagttcgaatttattgagagccagaccagcagccaccgtatcaacgcgttttggattggtctgagccgtaaccaaagcgagggtccgtggttctgggaagatggcagcgcgttctttccgaacagctttcaagtgcgtaacaccgcgccgcaagaaagcctgctgcacaactgcgtttggattcacggcagcgaggtttacaatcaaatctgcaataccagcagctacagcatctgcgagaaggaactggcttaattaa

SEQ ID NO: 2 - protein, 199 aa, Artificial sequence
  Synthetic construct
MAHHHHHHVGTGSGKGKGSGSGFWRHNSGRNPEEKDSFLSRNKENHKPTESSLDEKVAPSKASQTTGGFSQSCLPNWIMHGKSCYLFSFSGNSWYGSKRHCSQLGAHLLKIDNSKEFEFIESQTSSHRINAFWIGLSRNQSEGPWFWEDGSAFFPNSFQVRNAVPQESLLHNCVWIHGSEVYNQICNTSSYSICEKELA

SEQ ID NO: 3 - DNA, 574 bp, Artificial sequence
  Synthetic construct
atggcacatcaccaccaccatcacgtgggtaccggcagcggcaagggcaagggcagcggcagcggtataatggatcaaccctcaagaaggctatatgagctgcacacgtaccacagctccctcacgtgcttttctgagggtactatggtgtccgagaaaatgtggggctgctgcccgaatcattggaaatcttttggtagcagctgttatctgatcagcaccaaagagaacttctggagtaccagcgagcaaaactgcgtccagatgggcgcacacctggttgtgattaacaccgaagcggaacagaacttcatcacccagcaattaaatgaaagcttgtcttacttcctgggtctgtcggatccgcagggcaacggcaagtggcagtggattgacgacaccccgttctcccaaaacgtgcgcttttggcatccgcatgaaccgaatctgccggaagaacgttgtgtaagcattgtttattggaatccaagcaagtggggttggaacgacgttttttgtgatagcaagcacaactcgatctgcgagatgaaaaagatctacttggcttaattaa

SEQ ID NO: 4 - protein, 189 aa, Artificial sequence
  Synthetic construct
MAHHHHHHVGTGSGKGKGSGSGIMDQPSRRLYELHTYHSSLTCFSEGTMVSEKMWGCCPNHWKSFGSSCYLISTKENFWSTSEQNCVQMGAHLVVINTEAEQNFITQQLNESLSYFLGLSDPQGNGKWQWIDDTPFSQNVRFWHPHEPNLPEERCVSIVYWNPSKWGWNDVFCDSKHNSICEMKKIYLA

SEQ ID NO: 5 - DNA, 604 bp, Artificial sequence
  Synthetic construct
atggctcaccatcaccaccaccattatggtaccggttccggcaaaggcaagggctccggctctggtcactatttcctgcgttggacccgcggttccgtggtgaaactgagcgactaccatacgcgcgtgacttgcattcgtgaagagccgcagccgggcgcaaccggcggtacatggacgtgctgcccggttagctggcgtgcgttccagtctaactgttatttcccactgaatgacaaccaaacgtggcatgagagcgaacgtaactgcagcggcatgagcagtcacctggttaccattaacaccgaggcggagcaaaactttgtgacccaattgctcgacaagcgcttcagctacttcctgggtttggccgatgaaaatgttgagggtcagtggcagtgggtagataagaccccgtttaatccgcacaccgtcttttgggaaaagggtgagtcgaacgacttcatggaagaagattgtgttgttctggtgcacgtgcacgagaagtgggtttggaatgatttcccgtgtcattttgaagtcagacgtatctgcaaattaccgggtatcacctttaactggaaaccgagcaaagcttaattaa

SEQ ID NO: 6 - protein, 199 aa, Artificial sequence
  Synthetic construct
MAHHHHHHVGTGSGKGKGSGSGHYFLRWTRGSVVKLSDYHTRVTCIREEPQPGATGGTWTCCPVSWRAFQSNCYFPLNDNQTWHESERNCSGMSSHLVTINTEAEQNFVTQLLDKRFSYFLGLADENVEGQWQWVDKTPFNPHTVFWEKGESNDFMEEDCVVLVHVHEKWVWNDFPCHFEVRRICKLPGITFNWKPSKA

SEQ ID NO: 7 - DNA, 649 bp, Artificial sequence
  Synthetic construct
atggcacatcaccaccaccatcacgtgggtaccggttcgaatgatgacgacgacaagagtccggatcccggaagtggaaaaggcaagggttcagggtctggcatctggcgtagcaacagcggtagcaataccctggaaaacggttactttttgagccgcaacaaggagaaccattctcagccgacgcaaagcagcttggaggactcggtgaccccaacgaaagctgtgaagaccacgggtgtcctgtcatccccgtgcccgccgaattggatcatctatgagaaaagctgctatctctttagcatgagcctgaatagctgggacggctccaaacgtcagtgttggcagctgggctcgaacttgctgaagatcgactcctctaacgaattaggtttcattgttaagcaagttagcagccaaccggataattccttttggattggcctgtctcgtccgcagaccgaagttccgtggctgtgggaagatggttccactttcagctctaacctgttccagattcgcaccaccgcaacccaagagaatcctagtccgaactgcgtttggattcacgtgagcgtgatctacgatcagctgtgttccgtcccgagctactcgatctgcgagaaaaagttctctatggcttaattaa

SEQ ID NO: 8 - protein, 214 aa, Artificial sequence
  Synthetic construct
MAHHHHHHYGTGSNDDDDKSPDPGSGKGKGSGSGIWRSNSGSNTLENGYFLSRNKENHSQPTQSSLEDSVTPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSMA

SEQ ID NO: 9 - DNA, 616 bp, Artificial sequence
  Synthetic construct
atggcacatcaccaccaccatcacgtgggtaccggttcgaatgatgacgacgacaagagtccggatcccgggtctggaaaaggcaagggaagtggttcaggcacctaccactttacctacggcgaaaccggtaaacgtctgtccgagctccactcatatcactcctctctgacgtgctttagcgagggtactaaagtgccagcgtggggttgttgtccggcgagctggaagtcgttcggcagcagctgctatttcatcagctcggaggaaaaagtttggagcaagagcgagcaaaactgcgtggaaatgggtgcacatttggttgtcttcaacaccgaagcggagcaaaactttatcgtgcagcagctgaacgaaagcttctcctacttcctgggtctgtccgacccgcagggtaataacaactggcagtggattgataaaaccccgtatgaaaagaacgtgcgcttttggcatttgggcgagccgaatcattctgccgaacaatgtgcgagcattgttttctggaagccgaccggctggggttggaatgacgttatttgcgagacgcgtcgtaacagcatctgcgagatgaataaaatctacctggcttaattaa

SEQ ID NO: 10 - protein, 203 aa, Artificial sequence
  Synthetic construct
MAHHHHHHVGTGSNDDDDKSPDPGSGKGKGSGSGTYHFTYGETGKRLSELHSYHSSLTCFSEGTKVPAWGCCPASWKSFGSSCYFISSEEKVWSKSEQNCVEMGAHLVVFNTEAEQNFIVQQLNESFSYFLGLSDPQGNNNWQWIDKTPYEKNVRFWHLGEPNHSAEQCASIVFWKPTGWGWNDVICETRRNSICEMNKIYLA

SEQ ID NO: 11 - DNA, 628 bp, Artificial sequence
  Synthetic construct
atggctcaccatcaccaccaccattatggtaccggttcgaatgatgacgacgacaagagtccggatcccgggtctggaaaaggcaagggaagtggttcaggccacaacttcagccgttgtaagcgcggtacgggcgtgcataagttggagcaccacgccaagctcaagtgcatcaaagaaaaatccgagctgaaatctgctgagggcagcacctggaactgctgcccgattgattggcgtgcgtttcaaagcaattgctacttcccgctgaccgataacaaaacctgggcggaaagcgagcgcaactgcagcggtatgggtgcacatctgatgaccatttcgaccgaagcggagcagaatttcatcatccaatttttggaccgccgtctgtcctacttcctgggtctgcgtgatgaaaatgcaaaaggccaatggcgttgggttgaccagaccccgtttaacccgcgtcgtgttttttggcataagaacgaaccagacaacagccagggtgaaaactgcgtcgtgttagtttataaccaggataaatgggcgtggaacgacgtgccgtgtaatttcgaggcttctcgcatttgtaagatcccgggtacgactctgaatgcttaattaa

SEQ ID NO: 12 - protein, 207 aa, Artificial sequence
  Synthetic construct
MAHHHHHHVGTGSNDDDDKSPDPGSGKGKGSGSGHNFSRCKRGTGVHKLEHHAKLKCIKEKSELKSAEGSTWNCCPIDWRAFQSNCYFPLTDNKTWAESERNCSGMGAHLMTISTEAEQNFIIQFLDRRLSYFLGLRDENAKGQWRWVDQTPFNPRRVFWHKNEPDNSQGENCVVLVYNQDKWAWNDVPCNFEASRICKIPGTTLNA

SEQ ID NO: 13 - DNA, 1168 bp, Artificial sequence
  Synthetic construct
atggcacatcaccaccaccatcacgtgggtaccgggagcggcatggtatcaaaaggtgaagagttgttcaccggggtggttccgattttggttgaactggacggagatgtgaatggccacaaatttagtgttagcggcgagggtgagggcgacgctacctacggcaaattgaccttaaagcttatctgcaccaccggtaagctgccggttccgtggccgacgctggtaactacgctgggttacggcctgcagtgcttcgcacgttacccggatcatatgaaacaacatgatttcttcaaaagcgccatgccggagggttacgtgcaggaacgtaccattttttttaaggacgatggtaattataaaacccgtgcagaagtgaaattcgagggtgataccttggtcaaccgtattgaactgaaaggcattgacttcaaggaggacggcaacatcctgggccacaagttggaatataattataacagccataatgtctacattatggcgggttccgggtctggtagtggttctggttcaggcagcggctctggctcgggctctggttccggctcaggttctggcagcggttcgggtagcggcaagggtaagggttctggcagtggcagcggtttctggcgtcacaactccggcagaaacccggaagagaaagactcgttcctgagccgcaacaaagaaaaccacaaaccgacggaatccagcctcgatgagaaggtggcaccgagcaaggccagccagaccactggtggtttcagccaaagttgcctgcctaattggatcatgcacggcaagagctgttatctgtttagcttcagcggtaacagctggtacggttccaaacgtcattgtagccaactgggtgcgcacctgctcaagatcgacaactccaaggagtttgagttcatcgagtcgcaaaccagcagccaccgtatcaacgcgttttggattggtctgtcccgcaatcagagcgaaggaccgtggttttgggaagatggctccgcgtttttcccgaacagctttcaggttcgcaacgcggttccgcaggagtctctgttgcacaactgcgtgtggattcatggtagcgaggtttacaaccaaatctgtaataccagcagctatagcatctgcgaaaaagaactggcttaattaa

SEQ ID NO: 14 - protein, 387 aa, Artificial sequence
  Synthetic construct
MAHHHHHHVGTGSGMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYIMAGSGSGSGSGSGSGSGSGSGSGSGSGSGSGSGKGKGSGSGSGFWRHNSGRNPEEKDSFLSRNKENHKPTESSLDEKVAPSKASQTTGGFSQSCLPNWIMHGKSCYLFSFSGNSWYGSKRHCSQLGAHLLKIDNSKEFEFIESQTSSHRINAFWIGLSRNQSEGPWFWEDGSAFFPNSFQVRNAVPQESLLHNCVWIHGSEVYNQICNTSSYSICEKELA

SEQ ID NO: 15 - DNA, 955 bp, Artificial sequence
  Synthetic construct
atggcacatcaccaccaccatcacgtgggtaccggtagcggcgctgataagcaaaaaaatgggatcaaggctaattttaaaatccgtcacaatattgaggacggtggggtgcagttggcggaccactaccagcagaatactccgatcggtgatggtccggttctgttgccggataatcactacctgtcctaccagagcgcgctgtcgaaagacccgaatgaaaagcgcgatcacatggttctgcttgagttcgtcaccgcggccggtattaccctgggtatggatgaatcacgcggtagcggttctggttccggctccggcagcggcagcggctcgggctctggctccggttccggctctggctccggcagtggcagcggcagcggtaaaggcaagggttctggctccggttcaggattttggcgtcacaactccggtcgtaacccggaagaaaaagacagcttcttgagcagaaacaaagagaaccacaaaccgaccgaatcgagcttggacgagaaggtggcaccgagcaaggcgagccaaaccacgggtggtttctcccaaagctgcctgccaaactggattatgcacggcaaaagctgctatctgttttctttcagcggtaacagctggtatggtagcaagcgccattgtagccaactgggcgcacatctgctcaagatcgacaattctaaggagtttgaattcatcgagagccaaaccagcagccatcgtattaacgccttctggatcggcttgtcacgtaaccagagcgagggtccgtggttttgggaagatgggtctgcgtttttcccgaactcgttccaggttcgtaatgctgtgccgcaagaatctctgctgcataactgcgtttggatccacggttccgaggtgtataaccagatttgcaacacgagcagctacagcatttgtgaaaaagagctggcttaattaa

SEQ ID NO: 16 - protein, 316 aa, Artificial sequence
  Synthetic construct
MAHHHHHHVGTGSGADKQKNGIKANFKIRHNIEDGGVQLADHYQQNTPIGDGPVLLPDNHYLSYQSALSKDPNEKRDHMVLLEFVTAAGITLGMDESRGSGSGSGSGSGSGSGSGSGSGSGSGSGSGSGKGKGSGSGSGFWRHNSGRNPEEKDSFLSRNKENHKPTESSLDEKVAPSKASQTTGGFSQSCLPNWIMHGKSCYLFSFSGNSWYGSKRHCSQLGAHLLKIDNSKEFEFIESQTSSHRINAFWIGLSRNQSEGPWFWEDGSAFFPNSFQVRNAVPQESLLHNCVWIHGSEVYNQICNTSSYSICEKELA

SEQ ID NO: 17 - DNA, 1084 bp, Artificial sequence
  Synthetic construct
atggcacatcaccaccaccatcacgtgggtaccggcagcggcaagggcaagggcagcggcagcggtataatggatcaaccctcaagaaggctatatgagctgcacacgtaccacagctccctcacgtgcttttctgagggtactatggtgtccgagaaaatgtggggctgctgcccgaatcattggaaatcttttggtagcagctgttatctgatcagcaccaaagagaacttctggagtaccagcgagcaaaactgcgtccagatgggcgcacacctggttgtgattaacaccgaagcggaacagaacttcatcacccagcaattaaatgaaagcttgtcttacttcctgggtctgtcggatccgcagggcaacggcaagtggcagtggattgacgacaccccgttctcccaaaacgtgcgcttttggcatccgcatgaaccgaatctgccggaagaacgttgtgtaagcattgtttattggaatccaagcaagtggggttggaacgacgttttttgtgatagcaagcacaactcgatctgcgagatgaaaaagatctacttgggtagcggcagcggaagtggaggtggaggtggaagcggcagcggaatggtatcaaaaggtgaagagttgttcaccggggtggttccgattttggttgaactggacggagatgtgaatggccacaaatttagtgttagcggcgagggtgagggcgacgctacctacggcaaattgaccttaaagcttatctgcaccaccggtaagctgccggttccgtggccgacgctggtaactacgctgggttacggcctgcagtgcttcgcacgttacccggatcatatgaaacaacatgatttcttcaaaagcgccatgccggagggttacgtgcaggaacgtaccattttttttaaggacgatggtaattataaaacccgtgcagaagtgaaattcgagggtgataccttggtcaaccgtattgaactgaaaggcattgacttcaaggaggacggcaacatcctgggccacaagttggaatataattataacagccataatgtctacattatggcggcttaattaa

SEQ ID NO: 18 - protein, 359 aa, Artificial sequence
  Synthetic construct
MAHHHHHHYGTGSGKGKGSGSGIMDQPSRRLYELHTYHSSLTCFSEGTMVSEKMWGCCPNHWKSFGSSCYLISTKENFWSTSEQNCVQMGAHLVVINTEAEQNFITQQLNESLSYFLGLSDPQGNGKWQWIDDTPFSQNVRFWHPHEPNLPEERCVSIVYWNPSKWGWNDVFCDSKHNSICEMKKIYLGSGSGSGGGGGSGSGMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYIMAA

SEQ ID NO: 19 - DNA, 871 bp, Artificial sequence
  Synthetic construct
atggcacatcaccaccaccatcacgtgggtaccggcagcggcaagggcaagggcagcggcagcggtataatggatcaaccctcaagaaggctatatgagctgcacacgtaccacagctccctcacgtgcttttctgagggtactatggtgtccgagaaaatgtggggctgctgcccgaatcattggaaatcttttggtagcagctgttatctgatcagcaccaaagagaacttctggagtaccagcgagcaaaactgcgtccagatgggcgcacacctggttgtgattaacaccgaagcggaacagaacttcatcacccagcaattaaatgaaagcttgtcttacttcctgggtctgtcggatccgcagggcaacggcaagtggcagtggattgacgacaccccgttctcccaaaacgtgcgcttttggcatccgcatgaaccgaatctgccggaagaacgttgtgtaagcattgtttattggaatccaagcaagtggggttggaacgacgttttttgtgatagcaagcacaactcgatctgcgagatgaaaaagatctacttgggtagcggcagcggaagtggaggtggaggtggaagcggcagcggagctgataagcaaaaaaatgggatcaaggctaattttaaaatccgtcacaatattgaggacggtggggtgcagttggcggaccactaccagcagaatactccgatcggtgatggtccggttctgttgccggataatcactacctgtcctaccagagcgcgctgtcgaaagacccgaatgaaaagcgcgatcacatggttctgcttgagttcgtcaccgcggccggtattaccctgggtatggatgaatcacgcgcttaattaa

SEQ ID NO: 20 - protein, 288 aa, Artificial sequence
  Synthetic construct
MAHHHHHHYGTGSGKGKGSGSGIMDQPSRRLYELHTYHSSLTCFSEGTMVSEKMWGCCPNHWKSFGSSCYLISTKENFWSTSEQNCVQMGAHLVVINTEAEQNFITQQLNESLSYFLGLSDPQGNGKWQWIDDTPFSQNVRFWHPHEPNLPEERCVSIVYWNPSKWGWNDVFCDSKHNSICEMKKIYLGSGSGSGGGGGSGSGADKQKNGIKANFKIRHNIEDGGVQLADHYQQNTPIGDGPVLLPDNHYLSYQSALSKDPNEKRDHMVLLEFVTAAGITLGMDESRA

SEQ ID NO: 21 - DNA, 8 bp, Artificial sequence
  Synthetic construct
ttaattaa

SEQ ID NO: 22 - protein, 6 aa, Artificial sequence
  Synthetic construct
HHHHHH

SEQ ID NO: 23 - DNA, 6 bp, Artificial sequence
  Synthetic construct
ggtacc

SEQ ID NO: 24 - DNA, 6 bp, Artificial sequence
  Synthetic construct
ggatcc

(19) United States
(12) Patent Application Publication
(10) Pub. No.: US 2022/0331349 A1
(43) Pub. Date: Oct. 20, 2022

(54) TARGETED NANOPARTICLES AND THEIR USES RELATED TO FUNGAL INFECTIONS

(71) Applicant: UNIVERSITY OF GEORGIA RESEARCH FOUNDATION, INC., Athens, GA (US)

(72) Inventors: Richard B. Meagher, Athens, GA (US); Zachary Lewis, Athens, GA (US); Xiaorong Lin, Athens, GA (US); Michelle Momany, Athens, GA (US); Suresh Ambati, Athens, GA (US)

(73) Assignee: UNIVERSITY OF GEORGIA RESEARCH FOUNDATION, INC., Athens, GA (US)

(21) Appl. No.: 17/421,069

(22) PCT Filed: Jan. 8, 2020
(86) PCT No.: PCT/US2020/012763

§ 371 Date: Jul. 7, 2021

Related U.S. Application Data
(60) Provisional application No. 62/789,862, filed on Jan. 8, 2019.
(60) Provisional application No. 62/913,489, filed on Oct. 10, 2019.

(51) Int. Cl. A61K 31/7048; A61K 9/127; A61K 47/64; A61K 47/69; A61K 49/00; A61P 31/10

(52) U.S. Cl. CPC A61K 31/7048; A61K 9/1271; A61K 47/64; A61K 47/6911; A61K 49/0086; A61P 31/10

(57) ABSTRACT

Provided herein are targeted nanoparticles for the diagnosis, treatment or prevention of a fungal infection.

### CROSS-REFERENCE TO RELATED APPLICATIONS

This application is a U.S. National Phase under 35 U.S.C. § 371 of International Patent Application No. PCT/US2020/012763, filed Jan. 8, 2020, which claims priority to U.S. Provisional Application No. 62/789,862, filed Jan. 8, 2019, and U.S. Provisional Application No. 62/913,489, filed Oct. 10, 2019, both of which are hereby incorporated in their entireties by this reference.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy, and is hereby incorporated by reference into the specification. The name of the text file containing the Sequence Listing is UGA_007WO1_SEQ_LIST.txt. The text file is 35 KB, was created on Jul. 3, 2021, and is being submitted electronically via EFS-Web. The information the text file is identical to the sequence listing contained in the application.

### BACKGROUND

Hundreds of species of indigenous fungi cause a wide variety of diseases including aspergillosis, blastomycosis, candidiasis, coccidioidomycosis (valley fever), cryptococcosis, histoplasmosis, dermatomycosis, and Pneumocystis pneumonia (PCP), to name a few. Collectively pathogenic fungi infect many different organs, but skin and lungs are the most common site. Some fungal diseases are merely disabling, while others are life threatening. Despite advances in the understanding of the pathology of fungal infections, current methods for diagnosing and treating fungal infections are deficient.

### SUMMARY

Provided herein are targeted nanoparticles, for example, liposomes, for the diagnosis, treatment or prevention of a fungal infection. Some liposomes comprise an antifungal agent and a targeting molecule that binds a target antigen on a fungal cell. In the liposomes provided herein, the targeting molecule is incorporated into the outer surface of the liposome and the antifungal agent is encapsulated in the liposome.

Further provided is a method of treating or preventing a fungal infection in a subject comprising administering to the subject having a fungal infection or at risk of developing a fungal infection a plurality of liposomes, wherein each liposome in the plurality comprises an antifungal agent and a targeting molecule that binds a target antigen on a fungal cell, wherein the targeting molecule is incorporated into the outer surface of the liposome and the antifungal agent is encapsulated in the liposome.

Also provided is a method of making a plurality of liposomes comprising an antifungal agent and a targeting molecule that binds a target antigen on a fungal cell, wherein the targeting molecule is incorporated into the outer surface of each liposome and the antifungal agent is encapsulated in each liposome. The method comprises (a) dissolving or suspending the antifungal agent in solvent for about 10 minutes to about 30 minutes, at about 60° C., (b) encapsulating the antifungal agent into each liposome by mixing a plurality of liposomes in suspension with the antifungal/solvent solution of step a), for about 3 to about 5 hours, at about 60° C. or for about 24-120 hours, at about 37° C., and (c) incorporating the targeting molecule into the outer surface of each liposome by contacting the liposomes comprising the encapsulated antifungal agent with the targeting molecule conjugated to a lipid, for about 45 minutes to about 90 minutes, at about 60° C.

Also provided are liposomes comprising a targeting molecule that binds a target fungal antigen, wherein the targeting molecule is incorporated into the outer surface of the liposome, and wherein the targeting molecule is linked to a molecule that generates a signal when the targeting molecule binds the target fungal antigen. In some examples, the targeting molecule is linked to the C-terminal and/or an N-terminal fragment of a fluorescent protein or fragments of fluorescent proteins.

Further provided is a method for detecting a fungal infection in a subject or a sample from the subject comprising: a) contacting the subject or a sample from the subject with a plurality of liposomes, wherein each liposome in the plurality comprises a targeting molecule that binds a target fungal antigen, wherein the targeting molecule is incorporated into the outer surface of the liposome, and wherein the targeting molecule is linked to a molecule that generates a signal when the targeting molecule binds the target fungal antigen; and b) detecting a signal, wherein a signal indicates the presence of a fungal infection.

### DESCRIPTION OF THE DRAWINGS

The present application includes the following figures. The figures are intended to illustrate certain embodiments and/or features of the compositions and methods and to supplement any description(s) of the compositions and methods. The figures do not limit the scope of the compositions and methods, unless the written description expressly indicates that such is the case.

FIG. 1A shows a nucleic acid sequence encoding an exemplary codon optimized soluble mouse Dectin-1 (sDectin-1) (SEQ ID NO: 1). A vector pET-45b+ sequence of 9 codons is boxed with the start codon underlined. Sites for cloning into pET-45B+ KpnI (GGTACC)(SEQ ID NO: 23) and PacI (TTAATTAA) (SEQ ID NO: 21), respectively, are underlined. Codons for Gly Ser (G,S) flexible linker residues are shown in bold and codons for reactive lys (K) residues (AAG) are shown in bold, with lysine codons in italic). The mouse sDectin-1 sequence (CLEC7A, GenBank No. AAS37670.1) is shown in plain text; an Ala codon GCT and stop codons TAA and TTA are underlined, with stop codons in bold. Alternative gene name MmsDectin1lyshis. The length of the nucleotide sequence is 604 base pairs, with 597 base pairs encoding a protein of 199 amino acids in length. The nucleic acid encoding the exemplary codon-optimized mouse sDectin-1 was cloned into pET-45B+.

FIG. 1B shows the amino acid sequence (SEQ ID NO: 2) encoded by SEQ ID NO: 1. This is a polypeptide comprising a mouse sDectin-1 polypeptide. The N-terminal amino acid sequence and (His)₆ (HHHHHH) (SEQ ID NO: 22) affinity tag from pET-45B+ are boxed. The Gly Ser (GS) flexible linker residues and reactive lys (K) residues appear in bold with lysines in italic. Mouse sDectin-1 amino acid residues appear in plain text (amino acids 23-199 of SEQ ID NO: 2), ending in a C-terminal Ala residue (A) in bold, the codon for which was used to put stop codons and a PacI site in frame. It is understood that, optionally, a stop codon in any of the polypeptide sequences disclosed herein, if not part of the native polypeptide from which the polypeptide is derived, can be removed, to produce a polypeptide that does not include one or more stop codons. The protein comprising the mouse sDectin-1 polypeptide is 199 amino acids in length, with a molecular weight (MW) of 22,389.66 g/mole. The theoretical pI is 7.74. It is understood that any protein described herein comprising an affinity tag, for example, a (His)6 affinity tag, can be modified to remove the His tag. In some examples, any nucleotide sequence described herein can further comprise a protease cleavage site for post-translational and/or post-purification removal of the affinity tag.

FIG. 1C shows a nucleic acid sequence encoding an exemplary codon optimized soluble mouse Dectin-2 (sDectin-2) (SEQ ID NO: 3). The vector pET-45b+ sequence of 9 codons is boxed with the start codon underlined. Sites for cloning into pET-45B+ KpnI (GGTACC)(SEQ ID NO: 23) and PacI (TTAATTAA) (SEQ ID NO: 21), respectively, are underlined. Codons for Gly Ser (G,S) flexible linker residues appear in bold and the codons for reactive lys (K) residues (AAG) appear in bold, with lysine codons in italic. Codon optimized sDectin-2 from the CLEC6A mouse Dectin 2 gene appears in plain text, with an Ala codon (GCT) and stop codons, TAA and TTA, underlined and stop codons in bold. The alternative gene name is MmsDectin2lyshis. The length of the nucleic acid sequence is 574 base pairs, with 567 base pairs encoding a protein that is 190 amino acids in length. The nucleic acid encoding the codon-optimized mouse sDectin-2 exemplary was cloned into pET-45B+.

FIG. 1D shows the amino acid sequence (SEQ ID NO: 4) encoded by SEQ ID NO: 3. This polypeptide comprises a mouse sDectin-2 protein. The N terminal amino acid and (His)₆ (HHHHHH)(SEQ ID NO: 22) affinity tag from pET-45B+ is boxed. The Gly Ser (GS) flexible linker residues and reactive lys (K) residues appear in bold, with lysines in italic. Mouse sDectin-2 amino acid residues appear in plain text (amino acids 23-189 of SEQ ID NO: 4), ending in a C-terminal Ala residue (A) (bold), the codon for which was used to put stop codons and PacI site in frame. The polypeptide comprising the mouse sDectin-2 that is produced has 189 amino acids, with a MW of 21,699.25 g/mole and a theoretical pI of 6.33.

FIG. 1E shows a nucleic acid sequence encoding an exemplary codon optimized soluble mouse Dectin-3 (sDectin-3) (SEQ ID NO: 5). The vector pET-45b+ sequence of 9 codons is boxed with the start codon underlined. Sites for cloning into pET-45b+ KpnI (GGTACC) (SEQ ID NO: 23) and PacI (TTAATTAA) (SEQ ID NO: 21), respectively, are underlined. Codons for Gly Ser (G,S) flexible linker residues are shown in bold. Reactive lys (K) codons (AAG) are shown in bold, with lysines in italic. Codon optimized sDectin-3 from the CLEC4D mouse Dectin-3 gene (GenBank Accession No. NP_034949.3) is shown in plain text, with an Ala codon (GCT) and stop codons TAA and TTA underlined. Stop codons are shown in bold. The alternative gene name is MmsDectin3lyshis. The length of the nucleotide sequence is 604 base pairs, with 597 base pairs encoding a protein that is 199 amino acids in length. The nucleic acid encoding the exemplary codon-optimized mouse sDectin-3 was cloned into pET-45B+.

FIG. 1F shows the amino acid sequence (SEQ ID NO: 6) encoded by SEQ ID NO: 5. This polypeptide comprises a mouse sDectin-3 protein. The N terminal amino acid and (His)₆ (HHHHHH)(SEQ ID NO: 22) affinity tag from pET-45B+ is boxed. Gly Ser (GS) flexible linker residues and reactive lys (K) residues are shown in bold, with lysines in italic. Mouse sDectin-3 amino acid residues are shown in plain text (amino acids 23-199 of SEQ ID NO: 6), ending in a C-terminal Ala residue (A) in bold, the codon for which was used to put stop codons and PacI site in frame. The polypeptide is 199 amino acids in length with a MW of 23,023.72 g/mole and a theoretical pI or 6.52.

FIG. 1G shows a nucleic acid sequence encoding an exemplary codon optimized soluble human Dectin-1 (sDectin-1) (SEQ ID NO: 7). The human sDectin-1 DNA sequence is expressed from vector pET-45B+. The vector pET-45b+ sequence and His tag of 9 codons is boxed with the start codon underlined. Cloning sites BamHI (GGATCC)(SEQ ID NO: 24) and PacI (TTAATTAA)(SEQ ID NO: 21), respectively, are underlined. Codons for enterokinase processing site in lower case font, Codons for Gly Ser (G,S) flexible linker residues and reactive lys (K) residues (AAA and AAG) are shown in bold with lysine codons in italic. The human sDectin-1 sequence (CLEC7A, GenBank Accession No. NM_197947) is shown in plain text, codon optimized for expression. An Ala codon GCT and stop codons TAA and TTA underlined, with stop codons in bold. An alternate name for this sequence is HssDectin1lyshis. The nucleotide sequence encoding human sDectin-1 has a length of 649 base pairs, encoding a polypeptide that is 214 amino acids in length. The nucleic acid encoding the exemplary codon-optimized human sDectin-1 was cloned into pET-45B+.

FIG. 1H shows the amino acid sequence (SEQ ID NO: 8) encoded by SEQ ID NO: 7. This is a polypeptide comprising a human sDectin-1 protein. The N-terminal amino acid and (His)₆ (HHHHHH)(SEQ ID NO: 22) affinity tag from pET-45B+ is boxed. The enterokinase processing site in lower case font. The Gly Ser (GS) flexible linker residues and reactive lys (K) residues are shown in bold, with lysines in italic. Human sDectin-1 amino acid residues are shown in plain text (amino acids 35-214 of SEQ ID NO: 8), ending in a C-terminal Ala residue (A) in bold, the codon for which was used to put stop codons and PacI site in frame. The polypeptide is 214 amino acids in length, with a MW of 23,703.20 g/mole and a theoretical pI of 6.22.

FIG. 1I shows a nucleic acid sequence encoding an exemplary codon optimized soluble human Dectin-2 (sDectin-2) (SEQ ID NO: 9). The human sDectin-2 nucleotide sequence is expressed from vector pET-45B+. The length of the nucleotide sequence is about 580 base pairs with 616 base pairs encoding a protein of 203 amino acids in length. The vector pET-45b+ sequence of 9 codons including the His tag is boxed with the start codon underlined. Cloning sites BamHI (GGATCC)(SEQ ID NO: 24) and PacI (TTAATTAA)(SEQ ID NO: 21), respectively, are underlined. Codons for enterokinase processing site in lower case font. Codons for Gly Ser (G,S) flexible linker residues are shown in bold, and reactive lys (K) residues (AAG) are shown in bold, with lysines in italic. Codon optimized sDectin-2 from the CLEC6A human Dectin 2 gene (cDNA GenBank Accession No. NM_001317999) is shown in plain text. An Ala codon (GCT) and stop codons, TAA and TTA, are underlined, with stop codons shown in bold. The alternative gene name is HssDectin2lyshis. The nucleic acid encoding the codon-optimized human sDectin-2 exemplary was cloned into pET-45B+.

FIG. 1J shows the amino acid sequence (SEQ ID NO: 10) encoded by SEQ ID NO: 9. This polypeptide comprises a human sDectin-2 protein. The N-terminal amino acid and (His)₆ (HHHHHH)(SEQ ID NO: 22) affinity tag from pET-45B+ are boxed. The enterokinase processing site is in lower case font. Gly Ser (GS) flexible linker residues and reactive lys (K) residues are shown in bold, with lysines in italic. Human sDectin-2 amino acid residues are shown in plain text (GenBank Accession No. NP_001007034.1) (amino acids 36-203 of SEQ ID NO: 10), ending in a C-terminal Ala residue (A) in bold, the codon for which was used to put stop codons and PacI site in frame. The polypeptide is 203 amino acids in length, with a MW of 22,969 g/mole and a theoretical pI of 5.91.

FIG. 1K shows a nucleic acid sequence encoding an exemplary codon optimized soluble human Dectin-3 (sDectin-3) (SEQ ID NO: 11). The human sDectin-3 DNA sequence is expressed from vector pET-45B+ in E. coli. The vector pET-45b+ sequence of 9 codons with hist tag is boxed, with the start codon underlined. Sites for cloning into pET-45b+ BamHI (GGATCC)(SEQ ID NO: 24) and PacI (TTAATTAA)(SEQ ID NO: 21), respectively, are underlined. Codons for enterokinase processing site are in lower case font. Codons for Gly Ser (G,S) flexible linker residues are shown in bold and reactive lys (K) residues (AAG) are shown in bold, with lysines in italic. Codon optimized sDectin-3 from the CLEC4D human Dectin-3 gene, (GenBank Accession NM_080387) is shown in plain text. An Ala codon (GCT) and stop codons, TAA and TTA, are underlined, with stop codons in bold. The alternative gene name is HssDectin3lyshis. The nucleotide sequence has a length of 628 base pairs, encoding a polypeptide of 207 amino acids in length. The nucleic acid encoding the exemplary codon-optimized human sDectin-3 was cloned into pET-45B+.

FIG. 1L shows the amino acid sequence (SEQ ID NO: 12) encoded by SEQ ID NO: 11. This polypeptide comprises the human Dectin-3 protein. The N-terminal amino acid and (His)6 (HHHHHH)(SEQ ID NO: 22) affinity tag from pET-45B+ is boxed. The enterokinase processing site is in lower case font. The Gly Ser (GS) flexible linker residues and reactive lys (K) residues are shown in bold, with lysines in italic. The human sDectin-3 amino acid residues (GenBank Accession No. NP_525126) are shown in plain text (amino acids 35-207 of SEQ ID NO: 12), ending in a C-terminal Ala residue (A) in bold, the codon for which was used to put stop codons and a PacI site in frame. The protein is 207 amino acids in length with a MW of 23,662 g/mole and a theoretical pI of 7.64.

FIG. 1M shows a nucleic acid sequence encoding an exemplary codon optimized soluble mouse Dectin-1 (sDectin-1) fused to the N-terminal portion of Venus (SEQ ID NO: 13). It is understood that the soluble mouse Dectin-1 amino acid sequence that does not comprise a membrane domain is interchangeable with a soluble Dectin-1 amino acid sequence from another species, for example, a human Dectin-1 amino acid sequence as set forth in SEQ ID NO: 8 or a fragment thereof. Dectin-2 and Dectin-3 amino acid sequences, or fragments thereof, from mouse or other species can also be used in any of the constructs described herein to detect a fungal infection. MmsDECTIN1VyN is a codon optimized DNA sequence expressed in pET-45B+ encoding half of a BiFC diagnostic. Length: 1,168 base pairs, with 1,161 base pairs encoding a protein that is 385 amino acids in length. The vector pET-45b+ sequence of 9 codons is boxed with the start codon underlined. Cloning sites KpnI (GGTACC)(SEQ ID NO: 23) and PacI (TTAATTAA)(SEQ ID NO: 21), respectively, are underlined. The codons for short Gly Ser Gly flexible linker residues appear in bold, followed in lower case letters by a nucleotide sequences (465 base pairs) encoding the sequence for Venus VyN T154M, which is the N terminal half of a mutant Venus protein modified from GenBank Accession No. AKA95335, followed by a long GlySer spacer in bold with reactive lysines in italic, followed by a 528 residue long Mouse sDectin-1 sequence (CLEC7A, GenBank Accession No. AAS37670.1) in plain text, ending in stop codons TAA and TTA, in bold.

FIG. 1N shows the amino acid sequence (SEQ ID NO: 14) encoded by SEQ ID NO: 13. This polypeptide comprises a mouse MmsDectin1VyN protein. The N-terminal amino acid and (His)₆ (HHHHHH)(SEQ ID NO 22) affinity tag from pET-45B+ are boxed. The first Gly Ser (GS) flexible linker is shown in bold, followed by, in lower case letters the C-terminal portion of Venus fluorescent protein VyN, representing Venus residues 1-155 with mutation T154M (amino acids 15-169 of SEQ ID NO: 14). This is followed by a long GlySer spacer sequence that contains reactive lys (K) residues in italic for coupling to a lipid carrier, and then mouse sDectin-1 amino acid residues in plain text (amino acids 211-387 of SEQ ID NO: 14). The polypeptide is 387 amino acids in length with a MW of 42,181.62 g/mole and a predicted pI of 6.55.

FIG. 1O shows a nucleic acid sequence encoding an exemplary codon optimized soluble mouse Dectin-1 (sDectin-1) fused to the C-terminal portion of Venus (SEQ ID NO: 15). MmsDECTIN1VC is a codon optimized DNA sequence that can be expressed in pET-45B+ encoding half of a BiFC diagnostic. Length: 955 with 948 encoding a protein of 316 amino acids in length. The vector pET-45b+ sequence of 9 codons is boxed, with start codon underlined. Cloning sites KpnI (GGTACC)(SEQ ID NO: 23) and PacI (TTAATTAA)(SEQ ID NO: 21) at the start and the end, respectively, are underlined. The codons for Gly Ser (G,S) flexible linker residues are shown in bold. Then, the encoding sequence (252 nucleotides) for the C-terminal amino acid encoding sequence of Venus (a.a. residues 155 to 238, protein modified from GenBank Accession NoAKA95335 and containing the T154M mutation) is shown in lower case letters. Then, a long GlySer flexible linker, with reactive lysines in italic for coupling to a lipid carrier, are shown in bold. This is followed by a 528 residue long Mouse sDectin-1 sequence (CLEC7A, GenBank Accession No. NAAS37670.1) (shown in plain text) ending in stop codons TAA and TTA in bold.

FIG. 1P shows the amino acid sequence (SEQ ID NO: 16) encoded by SEQ ID NO: 15. This polypeptide comprises a mouse MmsDectin1VC protein. The N-terminal amino acid and (His)₆ (HHHHHH)(SEQ ID NO: 22) affinity tag from pET-45B+ are boxed. The first Gly Ser (GS) flexible linker sequence is shown in bold font. Then, in lower case letters, the C-terminal half of Venus fluorescent protein representing Venus residues 155 to 238 (amino acids 15-98 of SEQ ID NO: 16)(Genbank Accession AKA95335) is shown. Then, in bold, a long GlySer flexible spacer that contains reactive lys (K) residues in italic is shown. Mouse sDectin-1 amino acid residues are shown in plain text (amino acids 140-316), followed by a C-terminal Ala residue (A) used to put stop codons in frame. The polypeptide is 316 amino acids in length, with a MW of 34,081.21 g/mole and a predicted pI of 6.8.

FIG. 1Q. MmDEC2VyN also MmDEC2VyN CODON OPTIMIZED DNA SEQUENCE (SEQ ID NO: 17) expressed IN pET-45B. Length: 577 bp, Vector pET-45b+ sequence of 9 codons boxed with start codon underlined, cloning sites KpnI (GGTACC)(SEQ ID NO: 23) and PacI (TTAATTAA)(SEQ ID NO: 21), respectively, are underlined, Codons for Gly, Ser (G,S) flexible linker residues in bold and reactive Lys (K) residues AAG bold in italic, sDectin-2 sequence from the mouse Dectin 2 gene CLEC6A that was codon optimized for E. coli expression is in plain text, a Gly ser rich flexible spacer of 15 residues, followed in lower case letters by the 465 nucleotide long encoding sequence for Venus VyN T154M the N terminal half of a mutant Venus protein modified from AKA95335, an Ala (A) codon GCT and two stop codons TAA and TTA underlined, with stop codons in bold. Alternative gene name: MmsDectin2lyshis, Length: 1084 bp less 7 for termination and following cleavage site, Encoding 359 residue protein. But order from GenScript shorter 1057 bp version starting with KpnI site, GGTACC (SEQ ID NO: 23) to subclone into pET-45b+.

FIG. 1R. Final MmsDEC2VyN protein being synthesized (SEQ ID NO: 18). N terminal amino acid and (His)6 (HHHHHH)(SEQ ID NO: 22) affinity tag from pET-45B+ boxed, Gly Ser (GS) flexible linker residues and reactive lys (K) residues in bold with lysines in italic, 166 mouse sDectin-2 amino acid residues in plain text (amino acids 23-188 of SEQ ID NO: 18), gly ser rich flexible spacer of 15 residues, Venus residues 1-155 with mutation T154M (amino acids 204-359 of SEQ ID NO: 18), ending in a C-terminal Ala residue (A) in bold, the codon for which was used to put stop codons and PacI site in frame. 359 amino acids in total, MW 40,198 g/mole. pI 6.04. O.D. 280 1.940/mg/mL.

FIG. 1S. MmDEC2VC also MmDEC2VC CODON OPTIMIZED DNA SEQUENCE expressed IN pET-45B (SEQ ID NO: 19). Length: 577 bp, Vector pET-45b+ sequence of 9 codons boxed with start codon underlined, cloning sites KpnI (GGTACC)(SEQ ID NO: 23) and PacI (TTAATTAA)(SEQ ID NO: 21), respectively, are underlined, Codons for Gly, Ser (G,S) flexible linker residues in bold and reactive Lys (K) residues AAG bold in italic, sDectin-2 sequence from the mouse Dectin 2 gene CLEC6A that was codon optimized for E. coli expression is in plain text, a Gly ser rich flexible spacer of 15 residues, then in lower case letters the 252 nucleotide long encoding sequence for the C terminal amino acid encoding sequence of Venus (a.a. residues 155 to 238, protein modified from AKA95335, an Ala (A) codon GCT and two stop codons TAA and TTA underlined, with stop codons in bold. Alternative gene name: MmsDectin2lyshis. Length: 871 bp less 7 bp for termination codons and cleavage site, Encoding 288 residue protein. But order from Genscript shorter 844 bp version starting at KpnI site GGTACC (SEQ ID NO: 23) to subclone into pET-45b+.

FIG. 1T. Final MmsDEC2VC2 protein being synthesized (SEQ ID NO: 20). N-terminal amino acid and (His)6 (HHHHHH)(SEQ ID NO: 22) affinity tag from pET-45B+ boxed, Gly Ser (GS) flexible linker residues and reactive lys (K) residues in bold with lysines in italic, 166 mouse sDectin-2 amino acid residues in plain text (amino acids 23-188 of SEQ ID NO: 20), gly ser rich flexible spacer of 15 residues, Venus residues 155 to 238 (amino acids 204-288 of SEQ ID NO: 20), followed by two stop codons and PacI site in frame. 288 amino acids in total, MW 32,098 g/mole. pI 6.16, O.D. 280 2.02 OD/mg/mL.

FIG. 2 shows SDS PAGE analysis of soluble Dectin-1 (sDectin-1) in cell extracts and after purification. sDectin-1 protein was produced in the BL21 strain of E. coli grown in Luria Broth overnight from the pET-45B plasmid without IPTG induction, solubilized in GuHCl buffers, purified by Ni-NTA resin and examined by SDS PAGE. Extraction into buffers that also contained reducing agent beta mercaptoethanol and Triton-X100 detergent greatly increased recovery from insoluble inclusion bodies (center lanes) relative to buffers without them (right lanes). Protein examined on an 12% acrylamide gel stained with Coomassie Blue. Extraction of cells with urea buffers yielded very little protein.

FIG. 3 is a schematic of a model of DEC-AmB-LLs, liposomes loaded with sDectin-1, amphotericin B, and rhodamine. Amphotericin B (AmB, blue oval structure) was intercalated into the lipid bilayer of 100 nm diameter liposomes. sDectin-1 (DEC, green globular structure) was coupled to the lipid carrier DSPE-PEG and both DSPE-PEG-DEC and red fluorescent DHPE-Rhodamine (red star) were also inserted into the liposomal membrane. sDectin-1, Rhodamine, AmB and liposomal lipids are in a 1:2:13:100 mole ratio, respectively. Two sDectin-1 monomers (two DSEP-PEG-DEC molecules) must float together in the membrane to bind to cell wall beta-glucans (red sugar moieties). The two liposomal controls examined were BSA-AmB-LLs containing an equal ug amount of 65 kDa BSA in place of 22 kDa sDectin-1 (i.e., 0.33 BSA:2:13:100 mole ratio) and AmBisome-like liposomes (AmB-LLs) lacking any protein coating (0:2:13:100 mole ratio). From these mole ratios, the surface area of an 100 nm diameter liposome, and the published estimate of 5×10⁶ lipid molecules per 10⁶ nm² of lipid bilayer, it was estimated there are approximately 3,000 Rhodamine molecules in each liposome and about 1,500 sDectin-1 monmers in each DEC-AmB-LL.

FIGS. 4A-F show that sDectin-1 coated DEC-AmB-LLs bound strongly to swollen conidia and germ tubes of germinating A. fumigatus, while AmB-LLs did not. A. Rhodamine red fluorescent DEC-AmB-LLs bound swollen conidia (white arrows) and germlings germ tubes of A. fumigatus. B. Rhodamine red fluorescent AmBisome-like AmB-LLs did not bind. No liposomes were detected even when the red channel was enhanced as in this image. The smallest red dots represent individual 100 nm diameter liposomes viewed based on their fluorescence (orange arrows). Large clusters of liposomes form the more brightly red stained areas. C through F. C and D were stained with DEC-AmB-LLs. E and F stained with BSA-AmB-LLs. A and B. Cells were grown for 8 hr in VMM+1% glucose at 37° C. Labeling performed in liposome dilution buffer LDB for 60 min. All three liposomes preparations were diluted 1:100 such that liposomal sDectin-1 and BSA proteins were at final concentrations of 1 ug/100 uL. Germlings were viewed in the green channel alone for cytoplasmic EGFP expression and red channel for liposomes. A and B were photographed at 63× under oil immersion. C through F were photographed at 20× on an inverted fluorescence microscope.

FIGS. 5A-F show that sDectin-1 coated DEC-AmB-LLs bound swollen conidia and hyphae of mature A. fumigatus cells, while untargeted AmBisome-like AmB-LLs did not. A. fumigatus conidia were germinated and grown for 16 hr in VMM+1% glucose at 37° C. before staining with fluorescent liposomes. A. through D. were stained with rhodamine red fluorescent DEC-AmB-LL diluted 1:100 such that sDectin-1 was at 1 ug/100 uL, and E. and F. with the equivalent amount of red fluorescent AmB-LLs for 60 min. A. DIC image alone. B. Combined DIC and red fluorescence image. A and B. show that Rhodamine fluorescent DEC-AmB-LLs bound to swollen conidia (white arrows) and hyphae. C through F examined cytoplasmic green EGFP with red fluorescene of liposomes. The smallest red dots represent individual 100 nm liposomes visible due to their strong fluorescence (orange arrows). C and D show that nearly all conidia and most hyphae stained with DEC-AmB-LLs. E & F show that AmB-LLs did not bind. A and B were photographed at 63× under oil immersion and seven stacked images were merged. C through F were photographed at 20× on an inverted fluorescent microscope.

FIGS. 6A-F show that sDectin-1-coated liposomes (DEC-AmB-LLs) bound strongly to Candida albicans and Cryptococcus neoformans cells. A., C. and E. are bright field images of C. albicans strain Sc5314 and C. neoformans strain H99 labeled with DEC-AmB-LL diluted 1:100 in LDB. B., D. and F. are the combined bright field and red fluorescence images showing that rhodamine labeled DEC-AmB-LLs bind strongly to these cells. Plain, uncoated AmB-LLs did not bind detectably to these cells. A & B were photographed at 63× under oil immersion, C through F at 20× on an inverted fluorescent microscope.

FIG. 7. sDectin-1-coated DEC-AmB-LLs bound orders of magnitude more frequently to A. fumigatus than control AmB-LLs or BSA-AmB-LLs and binding was inhibited by a soluble beta-glucan. Samples of 4,500 A. fumigatus conidia were germinated & grown at 37° C. for 36 hours VMM+1% glucose, fixed in formalin or examined live, and incubated for 1 hr with 1:100 dilutions of liposomes in liposome dilution buffer. Unbound liposomes were washed out. Multiple fields of red fluorescent images were photographed at 20× as in FIGS. 4 and 5. Each photographic field contained approximately 25 swollen conidia and an extensive network of hyphae. A, B, C. Labeling formalin fixed cells. D, E, F. Labeling live cells. G, H, I. Inhibition of DEC-AmB-LL labeling of fixed cells by 1 mg/mL laminarin, a soluble beta-glucan, vs 1 mg/mL sucrose as a control. A, D, & G. The number of red fluorescent liposomes and clusters of liposomes were counted, averaged per field and plotted on a log 10 scale. The numerical average is indicated above each bar and on the vertical axis. Standard errors are shown. Example fields of liposomes used to construct the bar graphs are shown in B, C, E, F, H, and I.

FIGS. 8A-G show that DEC-AmB-LLs killed or inhibited the growth A. fumigatus far more efficiently than AmB-LLs at AmB concentrations near the ED50 for AmB. Samples of 4,500 A. fumigatus conidia were germinated & grown in 96 well microtiter plates in Vogel's Minimal Media (VMM+1% glucose) and treated at the same time with liposome dilution buffer or liposome preparations delivering the indicated concentrations of AmB to the growth media (A-D 3 uM AmB, E. 0.09 uM AmB, F. 0.18 uM AmB). Viability and growth were estimated using Cell Titer Blue (CTB) reagent (A and C), which measures total cellular esterase activity or by hyphal length (B & D) or by percent germination (E and F). Background fluorescence from wells with CTB in media, but lacking cells and liposomes was subtracted. Std. Errors are indicated. Inset photos in B and D show examples of the length of hyphae assayed for AmB-LLs and DEC-AmB-LL treated sample. One unit of hyphal length in B and D equals 5 microns. A and B and C and D compare the results from two complete biological replicate experiments with liposomes prepared and stored in different buffers. In A & B, liposomes were prepared in RN #5 buffer (0.1 M NaH2PO4, 10 mM Triethanolamine, pH 7.2, 1 M L-Arginine, 100 mM NaCl, 5 mM EDTA, 5 mM BME (beta-mercaptoethanol)), and C through F in RN #5 buffer (0.1 M NaH2PO4, 10 mM Triethanolamine, pH 7.2, 1 M L-Arginine, 100 mM NaCl, 5 mM EDTA, 5 mM BME), and cells were grown for 56 hr (C, D) or 36 hr (A, B, E, F). G. A dose response curve based on the percent germination of conidia. Conidia and liposomes delivering various concentrations of AmB were plated together in VMM+ Glucose in wells of a microtiter plate. After 8 hr at 37° C., the percent of conidia that had germinated was quantified.

FIGS. 9A-B show that DEC-AmB-LLs more effectively inhibited the growth of A. fumigatus cells than AmBisome-like AmB-LLs at AmB concentrations near and below the ED₅₀ for AmB based on cytoplasmic GFP fluorescence. Samples of 4,500 A. fumigatus conidia were germinated & grown in 96 well microtiter plates in Vogel's Minimal Media (VMM)+1% glucose and treated with liposome dilution buffer control or dilutions of AmB loaded liposome preparations delivering 2 uM AmB (A.) and 0.67 AmB (B.) and incubated for 36 hrs at 37° C. Cell integrity was assayed based on the amount of cytoplasmic green fluorescence from the EGFP reporter in the A1163 strain of A. fumigatus. Fluorescence background from wells with media, but lacking cells and liposomes, was subtracted. Liposomes were stored in RN #5 buffer before use and diluted with liposome dilution buffer LDB.

FIG. 10 shows that sDectin-1 coated DEC-AmB-LLs were less toxic to HEK293 cells than uncoated AmBisome-like AmB-LLs. Human Embryonic Kidney HEK293 cells were treated for 2 hours with liposomes, after which the liposomes were washed out. Then the cells were incubated for an additional 16 hrs and assayed. Liposomes delivered a total of 30 or 15 uM of AmB to the media. CellTiter Blue esterase assays estimated cell viability and survival.

FIG. 11 shows a schematic model of immobilized exemplary Dectin-coated liposomes for detection of fungal beta-glucan and mannan polysaccharides in a subject or a sample from a subject. DEC-BiFC-Ls are liposomes coated with 500 sDectin monomers (blue) fused to the N-terminal half of a green fluorescent protein Venus VyN155 (e.g. DEC-VN−) and with 500 sDectin monomers fused to the C-terminal half of Venus VC155 (DEC-VC−) that will rapidly recognize and bind fungal beta-glucans or mannans to form sDectin dimers, leading to an assembled Venus, which will produce a strong green Bimolecular Fluorescence Complementation (BiFC) signal. These liposomes will produce a green fluorescent signal only when they contact a fungal cell or released beta-glucan or mannan. When these liposomes are attached to an insoluble matrix via, for example, biotin-streptavidin binding, they can detect low concentrations of fungal polysaccharides in large volumes of serum, using standard fluorescence instrumentation.

FIG. 12 is a schematic of a model of DEC-HRP-Ls, Dectin coated liposomes, for detection of fungal beta-glucans and mannans. sDectin (DEC, green globular structure) and horse radish peroxidase (HRP, hexagon) were coupled to the lipid carriers DSPE-PEG or DSPE and inserted into the liposomal membrane. sDectin, HRP, PEG, and liposomal lipids are in about a 1:1:13:100 mole ratio, respectively. Two sDectin monomers (two DSEP-PEG-DEC molecules) must float together in the membrane to bind to cell wall beta-glucans or mannans (red sugar moieties). After these liposomes bind to a fixed sample of fungi, excess unbound liposome is washed out. Then, the substrates for HRP enzyme activity (4-chloro-1-naphthol and peroxide) are added. After addition of the substrates, an HRP generated purple precipitate is measured to determine the total amount of DEC-HRP-L bound and the total amount of fungal polysaccharide.

FIG. 13A shows the modified mouse sDectin-2 DNA MmsDectin2lyshis DNA sequence (SEQ ID NO: 3). The codon-optimized DNA sequence of MmsDECTIN2lyshis was cloned into in pET-45B. NCBI BankiT #MN104679. Length: 577 bp, Vector pET-45b+ sequence of 9 codons boxed with start codon underlined, cloning sites KpnI (GGTACC) (SEQ ID NO: 23) and PacI (TTAATTAA)(SEQ ID NO: 21), respectively, are underlined, Codons for Gly, Ser (G,S) flexible linker residues in bold and reactive Lys (K) residues AAG bold in italic, sDectin-2 sequence from the mouse Dectin 2 gene CLEC6A that was codon optimized for E. coli expression is in plain text, ending in an Ala (A) codon GCT and two stop codons TAA and TTA in bold within the PacI site. Length of coding sequence and two stop codons: 574 bp, encoding a 189 residue protein. Alternative gene name: MmsDectin2lyshis.

FIG. 13B shows the sDectin-2 (DEC2) protein synthesized in E. coli (SEQ ID NO: 4). N terminal amino acid peptide sequence and (His)6 (HHHHHH) (SEQ ID NO: 22) affinity tag from pET-45B+ boxed, Gly Ser (GS) flexible linker residues and reactive lys (K) residues in bold with lysines in italic, 166 mouse sDectin-2 amino acid residues in plain text, ending in a C-terminal Ala residue (A) in bold. 189 amino acids in total, MW 21,763.29 g/mole with theoretical pI of 6.33. The sDectin-2 sequence represents a.a. residues 44 to 209 from the native mouse Dectin-2 sequence. Alternative protein name: MmsDectin2lyshis protein.

FIG. 14 shows SDS PAGE analysis of purified sDectin-2. The crude E. coli BL21 extracts not expressing and expressing sDectin-2 and purified sDectin-2 protein were examined by SDS PAGE on an 12% gel stained with Coomassie Blue. Molecular weight markers and the approximate molecular weight of modified sDectin-2 of 22 kDa are indicated on the left.

FIG. 15 shows a model of sDectin-2-coated liposomes loaded with rhodamine and Amphotericin B. Amphotericin B (AmB, blue ovoid structure) was intercalated into the lipid bilayer of 100 nm diameter liposomes. sDectin-2 (DEC2, green globular structure) was coupled to the lipid carrier DSPE-PEG and both DEC2-PEG-DSPE and red fluorescent DHPE-Rhodamine (red star) were inserted into the liposomal membrane via their lipid moieties, DSPE and DHPE. sDectin-2, Rhodamine, AmB, and liposomal lipids are in a 1:2:11:100 mole ratio. Two sDectin-2 monomers (two DEC2-PEG-DSPE molecules) must float together in the membrane to bind to fungal mannans (red sugar moieties). From these mole ratios, the surface area of an 100 nm diameter liposome and the published estimate of 5×10⁶ lipid molecules per 10⁶ nm² of lipid bilayer, it was calculated that in each DEC2-AmB-LL there are approximately 1,500 sDectin-1 monomers, 3,000 rhodamine molecules, and 16,500 AmB molecules in each liposome.

FIGS. 16A-F shows sDectin-2 coated liposomes, DEC2-AmB-LLs, bound to the extracellular matrix associated with C. albicans cells of diverse morphologies. A. Yeast cells. Cells are highlighted by Differential Interference Contrast microscopy (green, DIC) and rhodamine fluorescently labeled DEC2-AmB-LLs in red. B & C. Pseudo-hyphal (Ps-Hyp) and hyphae (Hyp). Cells are highlighted via their endogenous GFP fluorescence. DEC2-AmB-LLs bound in large clusters to the extracellular matrix (Ex). D, E, F. Mature hyphae. D. Bright field microscopy showing extracellular matrix surrounding hyphae of stained cells in E. E. Combined bright field image and red fluorescence of liposomes. F. Additional image parallel to that in E reiterating a typical staining of the matrix. All cells were stained for 1 hr with a 1:200 dilution of DEC2-AmB-LL into LDB2 buffer (e.g., 0.5 μg sDectin-2/100 μL). The extracellular matrices stained (Ex+) or not stained or weakly stained (Ex−) with DEC2-AmB-LLs are indicated. Arrows arrows indicate individual liposomes. Photographs were taken at 63× magnification under oil immersion. Several independent fungal cell labeling studies gave similar images.

FIGS. 17A-G show DEC2-AmB-LLs bound to the extracellular matrices associated with C. neoformans and A. fumigatus cells. Rhodamine red fluorescent DEC2-AmB-LLs bound in large clusters to the extracellular matrices (Ex) and rarely to the cell walls of both species. A, B, C, & D. C. neoformans. Yeast cells were co-stained with DEC2-AmB-LLs and mouse monoclonal antibody 18B7 to capsular glucuronoxylomannan (GXM) and secondary goat anti-mouse antibody Alexa488 (green). All cells were stained for 1 hr with a 1:200 dilution of DEC2-AmB-LL into LDB2 (e.g., 0.5 μg sDectin-2/100 4). A. Bright field image of cells. B. Green fluorescent image of GXM-specific antibody stained cells. C. The merged fluorescent image of B & D. D. Red fluorescent DEC2-AmB-LLs. E, F, & G. A. fumigatus. E & F. Bright field and combined fluorescent image of germlings grown for 10 hr and stained with DEC2-AmB-LL. G. Mature hyphae grown for 24 hr. Both species were stained for 1 hr with a 1:200 dilution of DEC2-AmB-LL into LDB2 (e.g., 0.5 μg sDectin-2/100 4). The extracellular matrices stained (Ex+) or not stained or weakly stained (Ex−) with DEC2-AmB-LLs or did not stain with either 18B7 or DEC2-AmB-LL (Ex−/−) are indicated. Photographs were taken at 63× magnification under oil immersion (plates A-F) or at 20× (plate 2G). Three independent fungal cell labeling studies gave similar images.

FIG. 18A-D shows that DEC2-Rhod (Rhodamine labeled DEC2) and DEC2-AmB-LL bound with similar patterns to the exopolysaccharide matrices surrounding A. fumigatus hyphal cells. A. fumigatus conidia were germinated at low density on microscope chamber slides in VMM+1% glucose+0.5% BSA and grown for 24 hr at 37° C. and fixed cells before staining for one hour with rhodamine labeled DEC2 protein DEC2-Rhod or DEC2 coated liposomes. A & B. DEC2-Rhod. C & D. DEC2-AmB-LLs. Cells were photographed at 20× taking differential interference contrast images (DIC, Plates A & C) and combined DIC and red fluorescence images (Plates B & D). Because the cells were highly dispersed and we wished to show several example cells in one plate, these images are composites made from cell images taken from separate photographic fields and placed adjacent to one another (see dotted outline of cells that were moved into a common field). The images are representative of 90% of the fungal cell colonies examined.

FIGS. 19A-I show that sDectin-2-coated DEC2-AmB-LLs bound one to two orders of magnitude more efficiently to C. albicans, C. neoformans and A. fumigatus cells than control AmB-LLs. Dense fields of fixed fungal cells were incubated for 1 hr with 1:200 dilutions of liposomes in liposome dilution buffer LDB2 (e.g. 0.5 μg sDectin-2/100 4). Unbound liposomes were washed out after one hr. Multiple fields of red fluorescent images were photographed at 20× and the area of red fluorescence estimated in Image J. Examples of photographic images are shown to the right of the bar graphs. A, B, & C. C. albicans, D, E, & F. C. neoformans. G, H, & I. A. fumigatus. In A, D, & G, standard errors from the mean are presented and fold differences and p-values are indicated to distinguish the binding of DEC2-AmB-LLs from AmB-LLs.

FIGS. 20A-H show the specificity, stability, and rate of DEC2-AmB-LL binding. A, B, & C. Specificity of binding. DEC-AmB-LL labeling of C. albicans was inhibited by soluble yeast mannan, but not by sucrose or laminarin. Each polysaccharide was added at 10 mg/mL during a 1 hr staining procedure. D, E, & F. Stability of binding. The plates of C. albicans cells stained with DEC2-AmB-LL and control liposomes used to generate the data in FIG. 3A-C, were left in PBS, stored in the dark for 2 months, re-photographed, and the area of liposome binding was re-quantified. G & H. Rate of binding. Mature cultures of C. albicans composed of some pseudo-hyphae and hyphae grown on the surface of a 24-well microtiter plate in VMM+20% FBS, fixed, blocked and, treated with DEC2-AmB-LLs for the indicated times. In all three experiments DEC2-AmB-LLs were diluted 1:200 w/v LDB2 (0.5 μg in 100 4 sDectin-2) and washed 4 times with LDB2. Multiple red fluorescent images were taken at 20× magnification for each time point on an inverted fluorescent microscope and the average area of red fluorescent liposome staining was estimated. Standard errors from the mean are shown in A, D, G, & H. In A & D the numerical average and number of fields examined is indicated above each bar and on the vertical axis. In A and D, fold differences, and p-values are indicated for the performance of DEC-AmB-LLs relative to mannan inhibition (A) or relative to AmB-LLs (D). These results are representative of two biological replicates.

FIGS. 21A-D show sDectin-2 coated Amphotericin B-loaded liposome inhibition and killing of C. albicans, C. neoformans and A. fumigatus. A. C. albicans with DEC2-AmB-LLs. Cells in the pseudo-hyphal and early hyphal stage grown in RPMI media+0.5% BSA on 96 well polystyrene microtiter plates. Cells were treated for 30 min with liposomes delivering 1.0., 0.5, 0.25 and 0.12 μM AmB to the media as indicated, washed twice with media, grown for an additional 16 hrs, and then assayed for metabolic activity using CellTiter-Blue (CTB) reagent. B. C. neoformans with DEC2-AmB-LLs. C. neoformans cells were grown in liquid YPD media+0.5% BSA with vigorous shaking for 2 hours and treated for 4 hours or overnight with liposomes delivering 0.4, 0.2, or 0.1 μM AmB to the media as indicated. Cells were diluted, plated on YPD media, and colony forming units (CFUs) counted from multiple plates. C. A. fumigatus with DEC2-AmB-LLs. Conidia were germinated for 9 hr in VMM+ glucose+0.5% BSA in 96 well polystyrene microtiter plates, treated for 2 hr with liposomes delivering 0.5, and 0.25 μM AmB to the media as indicated, washed twice with media, grown overnight, and then assayed for metabolic activity using CTB reagent in RPMI lacking phenol red indicator+0.5% BSA. The control wells were overgrown with hyphae protruding out the media, and hence, had low metabolic activity and generated a lower signal even though there were more cells in these wells. D. A. fumigatus with DEC1-AmB-LLs. Assay conditions were similar to the assay in C, except liposomes were first diluted into LDB1 buffer (PBS+0.5% BSA+1 mM BME) prior to dilution into growth media. For CTB assays in A, C, and D, the fluorescence background from media incubated with CTB reagent was subtracted. Standard errors are shown for all values and fold differences and p-values were estimated comparing the performance of AmB-LLs to DEC2-AmB-LLs. Two or more biological replicates gave similar results.

FIGS. 22A-B show two immunosuppression models of pulmonary aspergillosis and time lines for examining the efficacy of Dectin-2 targeted antifungal-loaded liposomes. A. The steroid model. B. The leukopenic model.

FIG. 23 shows Dectin-targeted antifungal drug-loaded liposomes. Antifungal drug-loaded liposomes coated with, for example, the carbohydrate recognition domain of Dectin-1 or Dectin-2 bind specifically to invasive fungal cells and biofilms and actively deliver antifungal drugs to fungal cells. Targeted liposomes bind orders of magnitude more efficiently to fungal cells (right side) than un-targeted drug-loaded liposomes (e.g., AmBisome®) or detergent solubilized antifungal drug (left side). Hence, targeted liposomes have a lower relative affinity for animal cells and deliver much higher doses of antifungal drugs to fungal cells at lower total drug concentrations relative to untargeted liposomes. By reducing the effective dose required to kill fungal cells, they are less toxic to animal cells.

FIGS. 24A-B show Conidia from A. fumigatus strain CEA10 germinate rapidly to establish infection centers in the lungs of CD1 mice. On D0 (FIG. 21) CD1 Swiss were given an oropharyngeal inoculum of 2×10⁶ CEA10 conidia. On D2, 48 hours after infection, mice were euthanized and hand prepared thin sections (e.g., ˜0.5 mm thick) of the various lobes of infected lungs were fixed in formalin and stained for fungal chitin for one hour with calcofluor white. Epi-fluorescent photographic images were taken using a DAPI filter set Ex_{A360}/Em_{A470} on a Leica DM6000 compound microscope. A. Most sections showed one large infection center with hundreds of stained hyphae. Photo taken at 5× magnification. B. Infection centers were composed of clusters of hyphal extensions. No ungerminated conidia were observed. Photo taken at 20× magnification.

FIGS. 25A-D show that, based on the steroid mouse model of immunosuppression-mediated aspergillosis DEC2-AmB-LLs were significantly more effective at reducing fungal burden in the lungs than AmB-LLs. Immunosuppressed CD1 Swiss mice (steroid model FIG. 21A) were infected with 2×10⁶ A. fumigatus conidia CEA10 strain (Day 0) and treated the next day (Day 1) with DEC2-AmB-LLs or AmBisome®-like AmB-LLs liposomes delivering 0.2 mg AmB/kg mouse weight or treated with the buffer used to dilute the liposomes (Buffer Control) (see time line in FIG. 21A). Three mice from each treatment group that survived to Day 4 were euthanized and the fungal burden per lung determined by two independent methods. A-C. Colony Forming Units (CFUs). A. A bar graph comparing the average number of CFUs per lung for the three treatment groups. B and C are example photographic images taken at 20× magnification of the fields of cells examined for the AmB-LL and DEC2-AmB-LL treatment groups, respectively. CFUs were estimated by plating homogenized lung tissue on rich growth media, incubating the plates overnight, and counting the number of fungal cell micro colonies. Standard errors are indicated with a line and whisker. D. Relative quantity of rDNA. The Relative Quantity (RQ) of A. fumigatus rDNA intergenic spacer (IGS) was determined by qPCR on parallel samples of lung homogenate from the same three sets of three lungs assayed in FIG. 24A. The data were normalized to the level of A. fumigatus rDNA in the one Buffer Control mouse with the highest level (i.e., the RQ for this mouse is set to 1.0).

FIGS. 26A-B shows that based on the leukopenic mouse model of immunosuppression-mediated aspergillosis DEC2-AmB-LLs were significantly more effective at reducing fungal burden in the lungs than AmB-LLs. Immunosuppressed CD1 Swiss mice (model FIG. 21B) were infected with 5×10⁵ A. fumigatus conidia CEA10 strain (Day 0) and treated the next day with DEC2-AmB-LLs or AmBisome®-like AmB-LLs liposomes delivering 0.2 mg AmB/kg mouse weight or treated with the buffer used to dilute the liposomes (Control) (see time line in FIG. 21B). Three mice from each treatment group were euthanized and the fungal burden per lung determined by two independent methods. A. A. fumigatus Colony Forming Units (CFUs). A bar graph comparing the average number of CFUs per lung for the three treatment groups. Standard errors are indicated with a line and whisker. B. Relative quantity of fungal cell rDNA. The Relative Quantity (RQ) of A. fumigatus rDNA intergenic spacer (IGS) was determined by qPCR on parallel samples of lung homogenate from the same three sets of three lungs assayed in A. The data were normalized to the level of A. fumigatus rDNA in the one control mouse with the highest level (i.e., the RQ for this mouse is 1.0).

FIGS. 26A-B shows that based on the leukopenic mouse model of immunosuppression-mediated aspergillosis DEC2-AmB-LLs were two orders of magnitude more effective at reducing fungal burden in the lungs than AmB-LLs. Immunosuppressed CD1 Swiss mice (model FIG. 21B) were infected with 5×10⁵ A. fumigatus conidia CEA10 strain (Day 0) and treated the next day (Day 1) with DEC2-AmB-LLs or AmBisome®-like AmB-LLs liposomes delivering 0.2 mg AmB/kg mouse weight or treated with the buffer used to dilute the liposomes (Buffer Control) (see time line in FIG. 21B). Three mice from each treatment group were euthanized and the fungal burden per lung determined by two independent methods. A. A. fumigatus Colony Forming Units (CFUs). A bar graph comparing the average number of CFUs per lung for the three treatment groups. Standard errors are indicated with a line and whisker. B. Relative quantity of fungal cell rDNA. The Relative Quantity (RQ) of A. fumigatus rDNA intergenic spacer (IGS) was determined by qPCR on parallel samples of lung homogenate from the same three sets of three lungs assayed in A. The data were normalized to the level of A. fumigatus rDNA in the one control mouse with the highest level (i.e., the RQ for this mouse is 1.0).

FIGS. 27A-D shows DNA encoding sequences and protein sequences of Dectin-2 Venus-fusions for the BiFC detection of fungal mannans. A and C. DNA sequences of MmDEC2VyN and MmDEC2VC, respectively. B. and D. Protein sequences of DEC2-VyN and DEC2-VC, respectively.

A. MmDEC2VyN CODON OPTIMIZED DNA SEQUENCE expressed in pET-45B (SEQ ID NO: 17). Length: 577 bp, Vector pET-45b+ sequence of 9 codons boxed with start codon underlined, cloning sites KpnI (GGTACC)(SEQ ID NO: 23) and PacI (TTAATTAA) (SEQ ID NO: 21), respectively, are underlined, Codons for Gly, Ser (G,S) flexible linker residues in bold and reactive Lys (K) residues AAG bold in italic, sDectin-2 sequence from the mouse Dectin 2 gene CLEC6A that was codon optimized for E. coli expression is in plain text, a Gly ser rich flexible spacer of 15 residues, followed in lower case letters by the 465 nucleotide long encoding sequence for Venus VyN T154M the N terminal half of a mutant Venus protein modified from AKA95335, an Ala (A) codon GCT and two stop codons TAA and TTA underlined, with stop codons in bold. Alternative gene name MmsDectin2lyshis, Length: 1084 bp less 7 bp for termination and following cleavage site (i.g., 1077 bp), encoding a 359 residue protein. But what was ordered from GenScript was a shorter 1057 bp version starting with KpnI site. GGTACC (SEQ ID NO: 23) to subclone into pET-45b+.

B. Final DEC2-VyN protein being synthesized (SEQ ID NO: 18). N terminal amino acid and (His)₆ (HHHHHH) (SEQ ID NO: 22) affinity tag from pET-45B+ boxed, Gly Ser (GS) flexible linker residues and reactive lys (K) residues in bold with lysines in italic, 166 mouse sDecetin-1 amino acid residues in plain text, gly ser rich flexible spacer of 15 residues, Venus residues 1-155 with mutation T154M, ending in a C-terminal Ala residue (A) in bold, the codon for which was used to put stop codons and PacI site in frame. 359 amino acids in total, MW 40,198 g/mole. pI 6.04. O.D. 280 1.940/mg/mL.

C. MmDEC2VC also MmDEC2VC CODON OPTIMIZED DNA SEQUENCE expressed IN pET-45B (SEQ ID NO: 19). Length: 577 bp, Vector pET-45b+ sequence of 9 codons boxed with start codon underlined, cloning sites KpnI (GGTACC) (SEQ ID NO: 23) and PacI (TTAATTAA) (SEQ ID NO: 21), respectively, are underlined, Codons for Gly, Ser (G,S) flexible linker residues in bold and reactive Lys (K) residues AAG bold in italic, sDectin-2 sequence from the mouse Dectin 2 gene CLEC6A that was codon optimized for E. coli expression is in plain text, a Gly ser rich flexible spacer of 15 residues, then in lower case letters the 252 nucleotide long encoding sequence for the C terminal amino acid encoding sequence of Venus (a.a. residues 155 to 238, protein modified from AKA95335, an Ala (A) codon GCT and two stop codons TAA and TTA underlined, with stop codons in bold. Alternative gene name: MmsDectin2lyshis, Length: 871 bp less 7 bp for termination codons and cleavage site, Encoding 288 residue protein. But order from Genscript a shorter 844 bp version starting at KpnI site GGTACC (SEQ ID NO: 23) to subclone into pET-45b+. 11 Final DEC2-VC protein being synthesized (SEQ ID NO: 20). C terminal amino acid and (His)₆ (HHHHHH) (SEQ ID NO: 22) affinity tag from pET-45B+ boxed, Gly Ser (GS) flexible linker residues and reactive lys (K) residues in bold with lysines in italic, 166 mouse sDecetin-2 amino acid residues in plain text, gly ser rich flexible spacer of 15 residues, Venus residues 155-238, ending in an added C-terminal Ala residue (A) in bold, the codon this Ala residue was used to put stop codons and PacI site in frame. 288 amino acids in total, MW 32,098 g/mole. pI 6.16, O.D. 280 2.02 OD/mg/mL.

FIGS. 28A and B shows the protein design and model of liposomes coated with Dectin-2 fused to the complementary fragments of Venus protein for detection of fungal mannan containing polysaccharides. A. A model of the diagnostic liposome employed. Liposomes coated with sDectin-2 monomers (blue) fused to the N-terminal fragment of a green fluorescent protein Venus VyN155 (e.g. DEC2-VyN) and with sDectin-2 monomers fused to the C-terminal fragment of Venus VC (DEC2-VC) recognize and bind fungal mannans to form sDectin-2 dimers, leading to an assembled Venus protein, and a strong green Bimolecular Fluorescence Complementation (BiFC) signal. These fungal diagnostic liposomes produce a green fluorescent signal only when the contact mannans in the fungal cell wall, exopolysaccharide matrices, biofilms, or released soluble polysaccharides containing mannans. DEC2-VyN and DEC2-VC were each coupled to the lipid carrier DSPE-PEG and inserted into the liposomal membrane. The DEC2 coupled monomers were inserted into liposomes such that the total DEC2 protein and liposomal lipids were in a 1:100 mole ratio, or one mole percent DEC2. From this mole ratio and the surface area of an 100 nm diameter liposome and the published estimate of 5×10⁶ lipid molecules per 10⁶ nm² of lipid bilayer⁷⁸, we calculated that in each DEC2-BiFC reagent liposome there were approximately 1,500 DEC2 monomers (i.e., 750 DEC2-VyN and 750 DEC2-VC fusion proteins). See Table 2. These liposomes can be easily attached to an insoluble matrix, via for example, Biotin-Streptavidin binding (right side of diagram). In this configuration, the DEC2-BiFC reagent should detect extremely low concentrations of fungal mannan-containing polysaccharides released into serum, using standard fluorescence instrumentation. B. Design of Dectin-2 Venus fusion proteins. Two separate Dectin-2 fusion proteins were expressed in E. coli. One was fused to the N-terminus of a mutant Venus protein VyN and the other to the C-terminal fragment of Venus protein VC. These two well characterized complementary fragments of the Venus protein are known to assemble and produce a BiFC signal, when brought into proximity by the assembly of interacting carrier proteins (i.e., herein DEC2 dimers). Flexible (glyser)ₙgly spacers of different lengths separate functional domains to allow some independence of movement and function.

FIG. 29 shows that DEC2-BiFC reagent generates a mannan specific Venus green fluorescent signal. Each polysaccharide was added at 1 mg/mL to a column of wells in a 96 well microtiter plate during incubation with DEC2-BiFC regent liposomes for 2 hr at 23° C. The average background signal from incubation with Dextran was subtracted from all other samples. Fold differences are indicated between the expected target yeast alpha-mannan and the fungal beta-glucan and sucrose. Standard errors are indicated by a line and whisker.

FIGS. 30A-J show that DEC2-BiFC reagent liposomes generate an A. fumigatus cell-dependent green fluorescent signal. 4,500 conidia of A. fumigatus (CEA10 strain) were germinated & grown on lysine coated 24-well microtiter plates at 37° C. for 72 hours in VMM+1% glucose, fixed in formalin, and washed into LDB2 buffer. A-H. Cells were incubated with 1:100 dilution of liposomal DEC2-BiFC reagent in liposome dilution buffer LDB2. The final DEC2 protein concentration was ˜1 ug/100 uL. Fungal cell colonies were photographed at 20× magnification pairing. Pairs of brightfield images and merged fluorescent images are shown. I & J. Image showing control cells incubated with dilution buffer and the red channel was reduced to show the negligible background of fluorescence from the green channel. A, C, E, G, I—On the left are bright field images of A. fumigatus cell colonies. B, D, F, H, J—On the right are bright field images (red) merged with Venus green fluorescent protein image of the adjacent bright field images on the left. White arrows in 30B indicate rare hyphae that to not produce a signal.

### DETAILED DESCRIPTION

The following description recites various aspects and embodiments of the present compositions and methods. No particular embodiment is intended to define the scope of the compositions and methods. Rather, the embodiments merely provide non-limiting examples that are at least included within the scope of the disclosed compositions and methods.

Globally, over 300 million people worldwide are afflicted with a fungal infection. Some fungal diseases are acute and severe. Other fungal infections are recurrent and some are chronic. There are about 200,000, 400,000 and 1,000,000 cases annually of aspergillosis, candidiasis, and cryptococcosis, respectively, with alarmingly high mortality rates. Fungal infections of the skin, dermatomycosis, are the most common fungal infections. It may be estimated that 4 to 10 percent of the global population (e.g., more than 300 million people) have fungal infections of the feet (athlete's foot infections, Tina pedia) and, in particular, infections of the toe nails (onychomycosis), which may be disabling.

Aspergillus fumigatus and related Aspergillus species cause aspergillosis. Patients at the greatest risk of developing life-threatening aspergillosis have weakened immune systems, for example, from stem cell transplants or organ transplants or have various lung diseases, including tuberculosis, chronic obstructive pulmonary disease (COPD), cystic fibrosis, or asthma. Among immunocompromised patients, aspergillosis is the second most common fungal infection, after candidiasis. Additional costs associated with treating invasive aspergillosis are estimated at $40,000 per child and $10,000 per adult. Patients with aspergillosis are treated with antifungal drugs such as amphotericin B, itraconazole, voriconazole, fluconazole, and others. Even with antifungal therapy, however, one-year survival among immunocompromised patients with aspergillosis is only 25 to 60%. Furthermore, all known antifungal agents that treat aspergillosis are quite toxic to human cells (Allen et al. Antifungal agents for the treatment of systemic fungal infections in children. Paediatrics & Child Health 15:603-608 (2010)). Provided herein are targeted liposomes that improve antifungal drug delivery and enhance therapeutic efficacy of an antifungal agent against a broad range of fungal pathogens and/or the reduce toxicity of the antifungal agent when administered to the subject.

### Nanoparticles

Provided herein are nanoparticles for delivery of drugs to fungal cells. As used throughout, nanoparticles can be, but are not limited to, lipid nanoparticles, for example, liposomes or non-liposomal lipid nanoparticles (for example, lipid nanoparticles with a non-aqueous core (LNPs)), dendrimers, polymeric micelles, nanocapsules or nanospheres, to name a few. For example provided herein is a nanoparticle, for example, a liposome, comprising an antifungal agent and a targeting molecule that binds a target antigen on a fungal cell, wherein the targeting molecule is incorporated into the outer surface of the nanoparticle and the antifungal agent is encapsulated in the nanoparticle. The targeting molecules used to target nanoparticles, for example, liposomes, to fungi described herein can target drug-loaded nanoparticles of various compositions to fungal cells. Other examples include, but are not limited to, iron oxide nanoparticles, polysaccharide gel nanoparticles and silica nanoparticles.

As used herein, the term liposome refers to an aqueous or aqueous-buffered compartment enclosed by at least one lipid bilayer. Liposomes are capable of carrying aqueous solutions, compounds, drugs or other substances in the compartment, i.e, internal cavity or space, enclosed by at least one lipid bilayer. Liposomes can vary in size, i.e., diameter. For example, a liposome can have a size of about 1000 nanometers (nm) or less. For example, a liposome can have a size of about 50 nm to about 1000 nm, about 50 nm to about 900 nm, about 50 nm to about 800 nm, about 50 nm to about 700 nm, about 50 nm to about 600 nm, about 50 nm to about 500 nm, about 50 nm to about 400 nm, about 50 nm to about 300 nm, about 50 nm to about 200 nm, or about 50 nm to about 100 nm. Liposomes include liposomes comprising a compartment for encapsulation of an agent, for example, an antifungal agent, liposomes comprising a targeting molecule attached to or incorporated into the outside of the liposome and liposomes comprising an encapsulated antifungal agent. An encapsulated antifungal agent is an antifungal agent that is completely or partially located in the interior space of the liposome. For example, in any of the liposomes described herein, at least about 75%, 80%, 85%, 90%, 95% or 99% of the antifungal agent is incorporated into the interior space of the liposome or into the lipid bilayer of the liposome.

Any of the nanoparticles described herein, for example, liposomes, can contain about 1, 2, 3, 4, 5, 6, 7, 8, 9, 10, 11, 12, 13, 14, 15, 16, 17, 18, 19, 20 moles percent or greater of an antifungal agent relative to lipid. In other words, the nanoparticles can comprise a 1:100, 2:100, 3:100, 4:100, 5:100, 6:100, 7:100, 8:100, 9:100, 10:100, 11:100, 12:100, 13:100, 14:100, 15:100, 16:100, 17:100, 18:100, 19:100, 20:100 mole ratio of antifungal agent to liposomal lipid or greater. As used herein, mole ratio is the ratio between the amounts in moles of two components, for example, the ratio between the number of moles of the targeting molecule and the number of moles of lipid (targeting molecules: moles of lipid) or the number of moles of an antigungal agent and the number of moles of lipid (moles of antifungal agent: moles of lipid). And similarly, the nanoparticles can comprise a 0.002:100, 0.05:100, 0.1:100, 0.5:100, 1:100, 2:100, 3:100, 4:100, 5:100, 10:100, 15:100, 20:100, 25:100 mole ratio of targeting protein to liposomal lipid or greater.

Pluralities of two or more of any of the liposomes described herein are also provided. For example, a plurality of liposomes can comprise from about two to about 1×10¹⁴ (100 trillion) liposomes. For example, a plurality can have at least 100, 250, 500, 750, 1000, 5000, 10,000, 25,000, 50,000,100,000, 500,000, 1 million or more liposomes. Liposomes can be made by any suitable method known to or later discovered by one of skill in the art. In general, liposomes can be prepared by a thin film hydration technique followed by a few freeze-thaw cycles. Liposomal suspensions can also be prepared according to methods known to those skilled in the art. Exemplary methods for the preparation of liposomes are described in Akbarzadeh et al. (“Liposome: classification, preparation and applications,” Nanoscale Res. Lett. 8(1): 102 (2013)) which is hereby incorporated by reference in its entirety.

In general, a variety of lipid components can be used to make liposomes. These include neutral lipids that exist either in an uncharged or neutral zwitterionic form at physiological pH. Such lipids include, for example, diacylphosphatidylcholine, diacylphosphatidylethanolamine, ceramide, sphingomyelin, dihydrosphingomyelin, cephalin, and cerebrosides. Synthetic derivatives of any of the lipids described herein can also be used to make lipid nanoparticles. Lipid nanoparticles can also comprise a sterol, for example, cholesterol. Lipid nanoparticles can also comprise a cationic lipid which carries a net positive charge at about physiological pH. Such cationic lipids include, but are not limited to, N,N-dioleyl-N,N-dimethylammonium chloride (DODAC); N-(2,3-dioleyloxy)propyl-N,N—N-triethylammonium chloride (DOTMA); N,N-distearyl-N,N-dimethylammonium bromide (DDAB); N-(2,3-dioleoyloxy)propyl)-N,N,N-trimethylammonium chloride (DOTAP); 1,2-Dioleyloxy-3-trimethylaminopropane chloride salt (DOTAP.Cl); 3.beta.-(N—(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethyl-ammonium trifluoracetate (“DOSPA”), dioctadecylamidoglycyl carboxyspermine (DOGS), 1,2-dileoyl-sn-3-phosphoethanolamine (DOPE), 1,2-dioleoyl-3-dimethylammonium propane (DODAP), N,N-dimethyl-2,3-dioleyloxy)propylamine (DODMA), and N-(1,2-dimyristyloxyprop-3-yl)-N,N-dimethyl-N-hydroxyethyl ammonium bromide (DMRIE). Anionic lipids are also suitable for use in lipid nanoparticles described herein. These include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

In some examples, the liposome comprises phosphatidylcholine, phosphatidylethanolamine, phosphatidylserine, phosphatidylinositol, phosphatidic acid, phosphatidylglycerol, palmitoyloleoyl phosphatidylcholine, lysophosphatidylcholine, lysophosphatidylethanolamine, dimyristoylphosphatidylcholine (DMPC), dipalmitoylphosphatidylcholine (DPPC), dioleoylphosphatidylcholine, distearoylphosphatidylcholine (DSPC), dilinoleoylphosphatidylcholine, a 1,2-distearoyl-sn-glycero-3-phosphoethanolamine (DSPE) conjugated polyethylene glycol (DSPE-PEG), a sphingomyelin, cholesterol, or any combination thereof. In some embodiments, PEG can be PEG-molecular weight (MW500) to PEG-MW20000. In addition to being components of the liposomes described herein, any of the lipids described herein can be conjugated to a targeting molecule or a fragment thereof that binds an antigen on a fungal cell. In some examples, pegylated versions of any of the lipids described herein can be conjugated to a targeting molecule or a fragment thereof that binds an antigen on a fungal cell, for example, a fungal cell wall antigen or a fungal cell exopolysaccharide matrix antigen.

As used throughout, a targeting molecule is a molecule that has a binding affinity for an antigen on a fungal cell, optionally a specific binding affinity, and can include, but is not limited to, an antibody, a polypeptide, a peptide, an aptamer or a small molecule. As used throughout, an antigen on a fungal cell or a target fungal cell antigen can be any antigen associated with a fungal cell at any stage of the fungal cell cycle. For example, and not to be limiting, an antigen can be associated with the fungal cell (for example, an antigen embedded in the fungal cell wall, an antigen attached to the fungal cell wall or a fungal cell surface antigen). An antigen associated with a fungal cell can also be directly or indirectly bound to the fungal cell, for example, directly or indirectly bound to the fungal cell wall. An antigen can also be an antigen of a fungal exopolysaccharide matrix, for example, a biofilm, produced by the fungal cell or associated with the fungal cell. In some examples, the exopolysaccharide matrix is adherent to or bound to the fungal cell or population of fungal cells. It is understood that an exopolysaccharide matrix associated with a fungal cell can be, but is not necessarily, produced by the fungal cell or the population of fungal cells it is associated with. As used throughout, an antigen can be, but is not limited to a protein, a lipid or a carbohydrate.

As used throughout, polypeptide, protein and peptide are used interchangeably herein to refer to a polymer of amino acid residues. As used herein, the terms encompass amino acid chains of any length, including full-length proteins, wherein the amino acid residues are linked by covalent peptide bonds.

As used throughout, the term nucleic acid refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

In each case, where specific nucleic acid or polypeptide sequences are recited, embodiments comprising a sequence having at least 70% (e.g. 70%, 75%, 80%, 85%. 90%, 95%, 99%) identity to the recited sequence are also provided. Identity or similarity with respect to a sequence is defined as the percentage of amino acid residues in the candidate sequence that are identical (i.e., same residue) with the starting amino acid residues, after aligning the sequences and introducing gaps, if necessary, to achieve the maximum percent sequence identity. For example, polypeptide and nucleic acid sequences having at least 70% (e.g. 70%, 75%, 80%, 85%. 90%, 95%, 99%) identity to SEQ ID NOs: 1-20 are provided herein. Polypeptides and nucleic acid sequences that do not include the histidine tag and/or linker sequences set forth in SEQ ID NOs: 1-20 are also provided. Polypeptides and nucleic acid sequences having at least 70% (e.g. 70%, 75%, 80%, 85%. 90%, 95%, 99%) identity to polypeptides and nucleic acid sequences that do not include the histidine tag and/or linker sequences set forth in SEQ ID NOs: 1-20 are also provided herein. Nucleic acids encoding the polypeptides described herein are also provided. Methods of alignment of sequences for comparison are well known in the art. Optimal alignment of sequences for comparison can be conducted, for example, by the local homology algorithm of Smith and Waterman (Adv. Appl. Math. 2:482, 1970), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), by computerized implementations of these algorithms (e.g., GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Any of the polypeptides disclosed herein can comprise one or more conservative amino acid substitutions. As a non-limiting example, the list below summarizes possible substitutions often likely to be carried out without resulting in a significant modification of the biological activity of the corresponding variant:

1) Alanine (A), Serine (S), Threonine (T), Valine (V), Glycine (G), and Proline (P);

2) Aspartic acid (D), Glutamic acid (E);

3) Asparagine (N), Glutamine (Q);

4) Arginine (R), Lysine (K), Histidine (H);

5) Isoleucine (I), Leucine (L), Methionine (M), Valine (V) and

6) Phenylalanine (F), Tyrosine (Y), Tryptophan (W).

See also, Creighton, Proteins, W.H. Freeman and Co. (1984).

In making such changes/substitutions, the hydropathic index of amino acids may also be considered. The importance of the hydropathic amino acid index in conferring interactive biologic function on a protein is generally understood in the art (Kyte and Doolittle; (1982) J Mol Biol. 157(1):105-32). It is accepted that the relative hydropathic character of the amino acid contributes to the secondary structure of the resultant protein, which in turn defines the interaction of the protein with other molecules, for example, enzymes, substrates, receptors; DNA, antibodies, antigens and the like.

Any of the polypeptides provided herein, for example, a soluble Dectin-1, Dectin-2 or Dectin-3 monomer can comprise an N-terminal or C-terminal deletion or truncation. For example, 1, 2, 3, 4, 5, 6, 7, 8, 9, 10 or more amino acids can be deleted from the N-terminal or C-terminal end of any polypeptide provided herein an still retain at least one function, for example, dimerization with another soluble Dectin monomer. As set forth in the figures, the soluble Dectin monomer polypeptide sequences appear in plain text. As set forth above, these sequences that do not include the histidine tag or linker sequence can be truncated by deleting 1, 2, 3, 4, 5, 6, 7, 8, 9, 10 or more amino acids can be deleted from the N-terminal and/or C-terminal end of the polypeptide.

Compositions comprising any of the polypeptides described herein are also provided. For example, a composition comprising a polypeptide comprising a soluble Dectin-1, Dectin-2 or Dectin-3 monomer polypeptide or a fragment thereof is provided herein. Optionally, the polypeptide can comprise or consist of amino acids 23-199 of SEQ ID NO: 2, amino acids 23-189 of SEQ ID NO: 4, amino acids 23-100 of SEQ ID NO: 6, amino acids 35-214 of SEQ ID NO: 8, amino acids 36-203 of SEQ ID NO: 10, or amino acids 35-207 of SEQ ID NO: 12. Fragments of polypeptides comprising or consisting of amino acids 23-199 of SEQ ID NO: 2, amino acids 23-189 of SEQ ID NO: 4, amino acids 23-100 of SEQ ID NO: 6, amino acids 35-214 of SEQ ID NO: 8, amino acids 36-203 of SEQ ID NO: 10, or amino acids 35-207 of SEQ ID NO: 12 are also provided. For example, and not to be limiting, fragments comprising a deletion of about 1, 2, 3, 4, 5, 6, 7, 8, 9, 10 or more amino acids from the N-terminal or C-terminal end of a polypeptide comprising amino acids 23-199 of SEQ ID NO: 2, amino acids 23-189 of SEQ ID NO: 4, amino acids 23-100 of SEQ ID NO: 6, amino acids 35-214 of SEQ ID NO: 8, amino acids 36-203 of SEQ ID NO: 10, or amino acids 35-207 of SEQ ID NO: 12 are also provided. Optionally, the polypeptide is linked or conjugated to a fluorescent moiety, for example, and not to be limiting, rhodamine. The composition can comprise a buffer, for example, a renaturation buffer comprising from about 0.5M to about 1.5 M L-Arginine, as described in the Examples. For example, and not to be limiting, the compositions can comprise a buffer comprising between about 0.05 and 0.15 M NaH2PO4, between about 10 mM and 20 mM Triethanolamine, between about 0.5M and 1.5 M L-Arginine, between about 50 and 200 mM NaCl, between about 2.5 mM and 7.5 mM EDTA and between about 0.25 and 7.5 mM BME, at pH 7.2. Optionally, the compositions can comprise a buffer comprising about 0.1 M NaH2PO4, about 10 mM Triethanolamine, about 1 M L-Arginine, about 100 mM NaCl, about 5 mM EDTA and 5 mM BME, at pH 7.2. A kit comprising any of the compositions is also provided. Optionally, the kit comprises a denaturation buffer or reduction buffer, for example, a reduction buffer comprising beta-mercaptoethanol. Kits comprising any of the liposomes described herein are also provided.

As used throughout, the term antibody encompasses, but is not limited to, a nanobody, a whole immunoglobulin (i.e., an intact antibody) of any class, including polyclonal and monoclonal antibodies, as well as fragments of antibodies that retain the ability to bind their specific antigens. Also useful are conjugates of antibody fragments and antigen-binding proteins (single chain antibodies) as described, for example, in U.S. Pat. No. 4,704,692, the contents of which are hereby incorporated by reference in their entirety.

As used throughout, an aptamer is an oligonucleotide (single stranded DNA or single stranded RNA) or a peptide molecule that selectively bind to a target antigen. See, for example, Lakhin et al. “Aptamers: Problems, Solutions and Prospects,” Acta Naturae 5(4): 34-43 (2013); and Reverdatto et al., “Peptide aptamers: development and applications,” Curr. Top Med. Chem. 15(12): 1082-101 (2015)) hereby incorporated in their entireties by this reference.

As used herein, the terms specifically binds or selectively binds mean binding that is measurably different from a non-specific or non-selective interaction. Specific binding can be measured, for example, by determining binding of a molecule to a target antigen compared to binding of a control molecule. Specific binding can be determined by competition with a control molecule that is similar to the target antigen, such as an excess of non-labeled target antigen. In that case, specific binding is indicated if the binding of the labeled target to a probe is competitively inhibited by the excess unlabeled target antigen.

Optionally, from two targeting molecules to about 10,000 targeting molecules can be incorporated into the liposomes provided herein. For example, from about five to about one hundred, about five to about two hundred, about five to about three hundred, about five to about four hundred, about five to about five hundred, about five to about six hundred, about five to about seven hundred, about five to about eight hundred, about five to about nine hundred, about five to about one thousand, about five to about 1100, about five to about 1200, about five to about 1300, about five to about 1400, about five to about 1500, about five to about 1600, about five to about 1700, about five to about 1800, about five to about 1900, about five to about 2000, about five to about 2250, about five to about 2500, or about five to about 3000 targeting molecules, about five to about 3500, about five to about 4000 targeting molecules, about five to about 4500 targeting molecules, about five to about 5000 targeting molecules, about five to about 5500 targeting molecules, about 5 to about 6000 targeting molecules, about 5 to about 6500 targeting molecules, about 5 to about 7000 targeting molecules, about 5 to about 7500 targeting molecules, about 5 to about 8000 targeting molecules, about 5 to about 8500 targeting molecules, about 5 to about 9000 targeting molecules, about 5 to about 9500 targeting molecules or about 5 to about 10,000 can be incorporated into one or more liposomes described herein. In some examples, about two molecules to about 3,000 targeting molecules are incorporated into nanoparticles that are 100 nm in diameter. Those of skill in the art would not know how to calculate the number of targeting molecules that can be incorporated into a nanoparticle, for example between about two and 10,000 targeting molecules or greater depending on the size of the nanoparticle.

As used throughout, by incorporation of a targeting molecule into the outer surface of the liposome means that the targeting molecule is incorporated into the outer lipid bilayer of the liposome or attached to the liposome. Incorporation can occur by insertion or intercalation of the targeting molecule into the lipid bilayer. Attachment to a liposome can occur, for example, by affinity to a molecule incorporated into the outer lipid bilayer of the liposome. For example, the liposome can be coated with biotin (for example, DSPE-PEG-biotin inserted into the lipid bilayer) and the targeting molecule linked to streptavidin. Alternatively, the targeting molecule can be conjugated to the outer surface of the liposome. Targeting molecules can be conjugated to liposomes by a number of methods known in the art (e.g., Arruebo et al. “Antibody-Conjugated Nanoparticles for Biomedical Applications,” Journal of Nanomaterials vol. 2009, Article ID 439389 (2009)). Liposomes can also be conjugated to targeting molecules via a streptavidin/biotin bond, thiol/maleimide chemistry, azide/alkyne chemistry, tetrazine/cyclooctyne chemistry, and other click chemistries. These chemical handles are prepared either during phosphoramidite synthesis or post-synthesis. As used herein, the term click chemistry refers to biocompatible reactions intended primarily to join substrates of choice with specific biomolecules. Click chemistry reactions are not disturbed by water, generate minimal and non-toxic byproducts, and are characterized by a high thermodynamic driving force that drives it quickly and irreversibly to high yield of a single reaction product, with high reaction specificity.

The targeting molecule can bind to an antigen on one or more types of fungal cells or populations of fungal cells, including, but not limited to cells from animal fungal pathogens (e.g., human fungal pathogens) and plant pathogens. Examples of human fungal pathogens include, but are not limited to, Alternaria alternata, Aspergillus species such as A. fumigatus, Blastomyces species such as B. dermatitidis, Candida species such as C. albicans, C. glabrata, C. krusei, C. auris, Coccidioides species such as C. immitis and C. posadasii, Cryptococcus species such as C. gattii and C. neoformans, Histoplasma species such as H. capsulatum, Pneumocystis species such as P. jirovecii, Sporothrix species such as S. schenckii, Talaromyces marneffei (formerly Penicillium marneffei), and Trichophyton rubrum.

Examples of plant fungal pathogens include, but are not limited to, Aecidium glycines, Aecidium mori, Alternaria japonica, Alternaria padwickii, Alternaria triticina, Alternaria yaliinficiens, Amylostereum areolatum, Apiognomonia erythrostoma, Arkoola nigra, Balansia oryzae-sativae, Botryosphaeria berengeriana, Calonectria pseudonaviculata—Shoot blight of boxwood—Calonectria pseudonaviculata, Ceratocystis fagacearum, Chalara fraxinea, Chrysomyxa abietis, Chrysomyxa himalensis, Chrysomyxa rhododendri, Ciborinia allii, Claviceps fusiformis, Claviceps gigantea, Claviceps sorghi, Claviceps sorghicola, Cronartium flaccidum, Crumenulopsis sororia, Diaporthe vexans, Didymella fabae-Ascochyta fabae, Endophyllum kaernbachii, Exobasidium vexans, Gerwasia imperialis, Gerwasia mayorii, Gerwasia rosae, Gerwasia rubi, Gerwasia variabilis, Goplana dioscoreae, Gymnosporangium miyabei, Gymnosporangium yamadae, Hamaspora acutissima, Hamaspora australis, Hamaspora hashiokai, Hamaspora longissimi, Hamaspora rubi-sieboldii, Hamaspora sinica, Harpophora maydis, Hemileia vastatrix, Kuehneola japonica, Kuehneola loeseneriana, Lachnellula willkommii, Leptographium wingfieldii, Mainsia rubi, Melampsora capraearum, Melampsora larici-epitea, Melampsora larici-pentandrae, Melampsora larici-pentandrae, Monilia polystroma, Monilinia fructigena, Ochropsora ariae, Olivea tectonae, Ophiostoma longicollum, Peronospora digitalis, Peronospora radii, Phakopsora ampelopsidis, Phakopsora euvitis. Phakopsora meibomiae, Phakopsora pachyrhizi, Phoma tracheiphila, Phragmidium acuminatum, Phragmidium arcticum, Phragmidium arisanense, Phragmidium assamense, Phragmidium barclayi, Phragmidium bulbosum, Phragmidium butleri, Phragmidium formosanum, Phragmidium griseum, Phragmidium hiratsukanum, Phragmidium kamtschatkae,—Phragmidium nambuanum, Phragmidium pauciloculare, Phragmidium rosae-moschatae, Phragmidium rosae-multiflorae, Phragmidium rosae-rugosae, Phragmidium rubi-thunbergii, Phragmidium yamadanum, Phyllachora maydis, Pileolaria pistaciae, Pileolaria terebinthi, Plasmopara obducens, Pseudocercospora angolensis, Puccinia agrophila, Puccinia buxi, Puccinia erythropus, Puccinia gladioli, Puccinia glyceriae, Puccinia hemerocallidis, Puccinia horiana, Puccinia kuehnii, Puccinia mccleanii, Puccinia melanocephala, Puccinia miscanthi, Puccinia pittieriana, Puccinia psidii, Puccinia substriata, Puccinia veronicae-longifoliae, Pucciniastrum coryli, Setomelanomma holmii, Sphaceloma poinsettiae, Sporisporium pulverulentum, Sporisorium sacchari, Thecaphora solani, Thekopsora areolate, Urocystis agropyri, Uromyces gladioli, Uromyces nyikensis, Uromyces transversalis, and Uromycladium tepperianum. It is understood that, in addition to detecting, treating or preventing a fungal infection in a subject, any of the methods provided herein can be used to detect, treat or prevent a fungal infection in a plant. For example, and not to be limiting, a fungal infection can be detected, treated or prevented on the surface of leaves, stems, roots, petals, sepals, stamens, carpels, and seeds or crushed samples or extracts from a plant.

In some examples, the targeting molecule is a C-type lectin receptor or a fragment thereof. In other examples, the targeting molecule is a chitin binding protein or a fragment thereof. Fragments of the targeting molecules described herein are polypeptides that can bind to a target antigen on a fungal cell with the same or a different binding affinity as the protein or polypeptide from which the fragment was derived. Examples of C-type lectin receptors that can be used as targeting molecules include Dectin-1 (CLEC7A, mouse GenBank Accession No.: AAS37670 and human GenBank Accession No.: NP_922938), Dectin-2 (CLEC6A mouse Genbank Accession No.: NP_064385 and human GenBank Accession No.: Q6EIG7), Dectin-3 (CLEC4D mouse GenBank Accession No.: NP_034949 and human GenBank Accession No.: NP_034949) and fragments of Dectin-1, Dectin-2, Dectin-3. Fragments of Dectin-1, Dectin-2, Dectin-3 comprising an amino acid sequence that binds to beta-glucans and/or mannans on a fungal cell can also be used as targeting molecules. Exemplary targeting molecules include but are not limited to SEQ ID NO: 2, which comprises the amino acid sequence of a mouse Dectin-1 beta-glucan binding fragment. SEQ ID NO: 2 comprises an N-terminal His tag, which can be removed. Any of the nucleic acid constructs used to make a soluble Dectin-1 (for example, SEQ ID NO: 7-14) can further comprise a selective protease cleavage site to remove the N-terminal His tag sequence after expression of the Dectin-1 polypeptide. These include, but are not limited to peptide protease processing sites recognized by tobacco etched virus (TEV) protease, enteropeptidase, thrombin, Factor Xa, and Rhinovirus 3C protease. Another exemplary targeting molecule is a soluble Dectin-3 polypeptide comprising SEQ ID NO: 6. SEQ ID NO: 6 comprises amino acids 44 to 219 of mouse Dectin-3. Additional exemplary targeting molecules include soluble human Dectin-1, Dectin-2 and Dectin-3 polypeptides comprising SEQ ID NO: 8, 10, and 12, respectively. SEQ ID NOs: 8, 10, and 12 comprise amino acids 69 to 248 of human Dectin-1, amino acids 42 to 209 of human Dectin-2 and amino acids 44 to 215 of human Dectin-3, respectively. Fragments of amino acids 69 to 248 of human Dectin-1, amino acids 42 to 209 of human Dectin-2 and amino acids 44 to 215 of human Dectin-3, for example, fragments comprising a deletion of 1, 2, 3, 4, 5, 6, 7, 8, 9, 10 or more amino acids from the C-terminal and/or N-terminal end of a polypeptide comprising or consisting of amino acids 69 to 248 of human Dectin-1, amino acids 42 to 209 of human Dectin-2 and amino acids 44 to 215 of human Dectin-3 are also provided and can be used in any of the liposomes, polypeptides or compositions described.

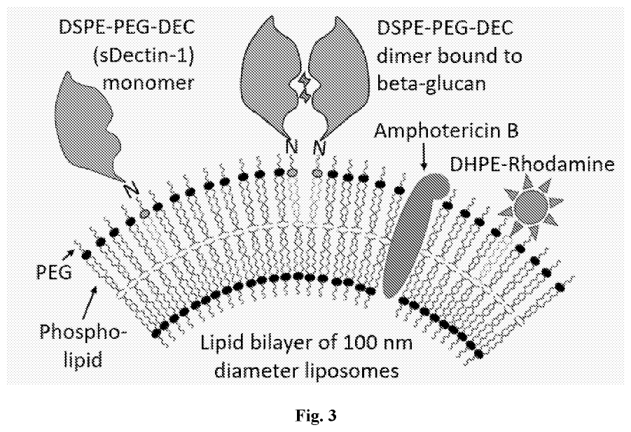

Dectin-1 is a transmembrane receptor expressed in T cells and is encoded by the CLEC7A (C-Type Lectin Domain Containing 7A, beta-Glucan Receptor, GR) gene in mice and humans. Dectin-1 binds various beta-glucans in fungal cell walls and is the primary receptor for transmembrane signaling the presence of exposed cell wall components on the surface of pathogenic and non-pathogenic fungi to stimulate an innate immune response. Human and mouse Dectin-1 are 244 and 247 amino acid-long plasma membrane proteins, respectively, although there are mRNA splice variants producing shorter human isoforms. Dectin-1 floats in the membrane as a monomer, but binds to beta-glucans as a dimer as modeled in the design of Dectin-1 targeted liposomes shown in FIG. 3. The 176 amino acid long (20 kDa) extracellular C-terminal domain can be manipulated alone as soluble sDectin-1 and contains the beta glucan binding domain. The beta 1→3 glucans are a structurally diverse class of polysaccharides, and as such, sDectin-1 binds various beta-glucans differentially with Kd affinity constants ranging from 2.6 mM to 2.2 pM. Having pan-fungal binding activity, Dectin-1 and fragments thereof, for example, a beta glucan binding fragment is an exemplary targeting molecule than can be used to kill one or more types of fungal cells, for example, and not to be limiting, Aspergillus, Candida and/or Cryptoccocus cells.

Other mammalian proteins with a mannan and manose binding domains that can be used for fungal polysaccharide targeting of liposomes are Mannose Receptor C type I (human MRC1, NG_0470), Mannose binding protein 2 (MBL2, NG_033955), and C-Type Lectin Domain Family 4 Member L (CD209, CLEC4L, or DC-SIGN, human NG_012167), or fragments thereof. Examples of non-mammalian proteins with fungal carbohydrate binding domains that can be used as targeting molecules include, but are not limited to, the bacterial and insect glucan binding proteins CBM11 (e.g., TYP77495 from Paenibacillus methanolicus) and CBM39 (e.g., EZA53410 from Ooceraea biori), bacterial mannan binding proteins CBM27 (e.g., PWV98652 from Paenibacillus cellulosilyticus), CBM35 (e.g., PYE65467 from Paenibacillus sp. OV191), CBM46 (e.g., GBF77546 from Paenibacillus sp. 598K) and MVL (e.g., AZB35797 from Chryseobacterium bernardetii) or fragments thereof.

Examples of mammalian proteins with chitin binding domains that can be used as targeting molecules include, but are not limited to, Chitinase-1 (CHIT1, human NG_012867), Chitinase-3 like-1 (HCGP39, CHI3L1, human NG_013056), Chitinase-3 like-2 (CHI3L2, human NM_001025197), Chitinase Acidic (CHIA, human gene care ID GC01P111291), and Chitobiase (CTBS, human gene card GC01M084549) or fragments thereof. Examples of non-mammalian proteins with chitin binding domains that can be used as targeting molecules include, but are not limited to, bacterial CBM5 (e.g., TDX99194 from Lysinibacillus xylanilyticus) and plant and/or fungal CBM18 (e.g., AYU56549 from Verticillium alfalfae) and CBM19 (e.g., XP_018290979 from Phycomyces blakesleeanus NRR1555).

Antifungals that can be incorporated or encapsulated in the targeted liposomes described herein include, but are not limited to a polyene or an azole antifungal. Examples of polyene antifungals include, but are not limited to, amphotericin B (AmB), candicidin, filipin, hamycin, natamycin, nystatin, hitachimycin and rimocidin. Examples of azole antifungals include, but are not limited to, imidazoles (for example, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, and Tioconazole), triazoles (for example, Albaconazole, Efinaconazole, Epoxiconazole, Fluconazole, Isavuconazole, Itraconazole, Posaconazole, Propiconazole, Ravuconazole, Terconazole, Voriconazole

thiazoles (for example, abafungin) and echinocandins (for example, caspofungin, micafungin and anidulafungin).

Amphotericin B (AmB) is the most commonly used agent for many kinds of fungal infections, including aspergillosis. The side effects of Amphotericin B include neurotoxicity and/or nephrotoxicity and/or hepatoxicity and often result in death of the patient. AmB is hydrophobic and is intercalated into the lipid bilayer of liposomes as modeled in FIG. 3. Commercial untargeted spherical AmB loaded liposomes, AmB-LLs, are often referred to as AmBisomes. AmB-LLs penetrate more efficiently to various organs, penetrate the cell wall and show reduced toxicity at slightly higher, more effective doses of AmB than the second most commonly used AmB product, deoxycholate detergent solubilized AmB. However, AmB-LLs still produce AmB human toxicity, such as renal toxicity in 50% of patients. When infected mice are treated with AmB-LLs, large fungal cell populations often remain. This large residual population is likely a reason that detergent solubilized AmB and AmB-LL treated human patients have high rates of recurrence and subsequent mortality after treatment. The targeted liposomes provided herein are designed to effectively target fungal cells and/or reduce toxicity of the antifungal agent, for example, AmB.

In some examples, the concentration of the antifungal drug is reduced as compared to the concentration of the antifungal drug incorporated into or encapsulated in a liposome that does not comprise a targeting molecule or a fragment thereof incorporated into the outer surface of the liposome, wherein the targeting molecule binds an antigen on a fungal cell. By forming protein-coated liposomes, i.e., coating the liposomes with a polypeptide, for example, a C-lectin type receptor or a fragment thereof, lower concentrations of the antifungal agent can be used to treat or prevent a fungal infection, thus decreasing the toxicity associated with administration of the antifungal drug to the subject. Lower toxicity allows extended use of the targeted antifungal agent over longer periods, which would reduce the fungal load beyond the poor reduction that is currently achieved. Lower toxicity could allow the targeted liposomes described herein to be used prophylactically, for example, as a nasal spray, to prevent lung infections before they are established.

In some liposomes, the reduction or decrease in the concentration of the antifungal drug is at least about 5%, 10%, 20%, 30%, 40%, 50%, 60%, 70%, 80%, 90%, 95% or any percentage in between these percentages. In some examples, the reduction in toxicity is a reduction in kidney cell toxicity and/or liver cell toxicity in vitro and/or in vivo. In another example, the reduction in the concentration of AmB in the liposome is reduced from about 11 moles percent relative to liposomal lipid to about 1 to 10 moles percent relative to liposomal lipid. In some targeted liposomes, the concentration of the antifungal agent can range from about 1 to about 20 moles percent antifungal agent relative to percent liposomal lipid. For example, the concentration of the concentration of the antifungal agent can be about 1, 2, 3, 4, 5, 6, 7, 8, 9, 10, 11, 12, 13, 14, 15, 16, 17, 18, 19 or 20 moles percent antifungal agent relative to percent liposomal lipid.

In some examples, the targeted liposome has decreased affinity for and/or is less toxic to an animal cell, for example, a human cell, as compared to a liposome that does not comprise a targeting molecule that binds an antigen on a fungal cell, for example, a targeting molecule incorporated into the outer surface of the liposome. In some examples, the targeted liposomes have a higher affinity for fungal cells in the lungs of a subject as compared to the affinity of the targeted liposomes for lung, kidney or liver cells of the subject. Therefore, the liposomes provided herein can be used to deliver an antifungal agent to a subject while minimizing the effects of the antifungal agent on non-fungal cells, for example, human lung, kidney or liver cells, thereby reducing the toxicity of the antifungal agent. Any of the liposomes comprising an antifungal agent described herein can be used to reduce or decrease fungal infection in vitro, ex vivo or in vivo.

### Methods for Making Targeted Liposomes

Provided herein is a method of making a plurality of liposomes comprising an antifungal agent and a targeting molecule that binds a target antigen on a fungal cell, wherein the targeting molecule is incorporated into the outer surface of each liposome and the antifungal agent is encapsulated in each liposome, the method comprising the steps of (a) dissolving the antifungal agent in solvent for about 10 minutes to about 30 minutes, at about 60° C. (b) encapsulating the antifungal agent into each liposome by mixing a plurality of liposomes in suspension with the antifungal/solvent solution of step (a), for about 3 to about 5 hours, at about 60° C. or at about 37° C. for about 24-120 hours; and (c) incorporating the targeting molecule into the outer surface of each liposome by contacting the liposomes comprising the encapsulated antifungal agent with the targeting molecule conjugated to a lipid, for about 45 minutes to about 90 minutes, at 60° C.

In the methods of making liposomes provided herein, the antifungal agent can be dissolved in any suitable solvent. Depending on the antifungal and its properties, one of skill in the art would know how to select an effective solvent for dissolution. In some examples, the antifungal agent, for example, amphotericin B is dissolved in aqueous DMSO or formamide. Other hydrophobic or amphiphobic solvents can also be used. Optionally, the antifungal agent can be dissolved for about 10 to about 120 minutes. For example, the antifungal agent can be dissolved for about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119 or 120 minutes.

Optionally, the antifungal agent can be dissolved at a temperature of about 55° C. to about 65° C., for example, at about 55, 56, 57, 58, 59, 60, 61, 62, 63, 64 or 65 degrees Celsius. Optionally, the antifungal agent is encapsulated into each liposome by mixing a plurality of liposomes in suspension with the antifungal/solvent solution (dissolved antifungal agent) for about 3 to about 5 hours, at about 55° C. to about 65° C., for example, at about 55, 56, 57, 58, 59, 60, 61, 62, 63, 64 or 65 degrees Celsius. In another example, the antifungal agent is encapsulated into each liposome by mixing a plurality of liposomes in suspension with the antifungal/solvent solution (dissolved antifungal agent) at about 35° C. to about 45° C., for example, at about 35, 36, 37, 38, 39, 40, 41, 42, 43, 44, or 45 degrees Celsius, for about 24-120 hours. In another example, the antifungal agent is encapsulated into each liposome by mixing a plurality of liposomes in suspension with the antifungal/solvent solution (dissolved antifungal agent) at about 35° C. to about 45° C., for example, at about 35, 36, 37, 38, 39, 40, 41, 42, 43, 44, or 45 degrees Celsius, for about 72-100 hours.

Optionally, in any of the methods for making liposomes described herein, the C-type lectin receptor or fragment thereof selected from the group consisting of Dectin-1, Dectin-2 and Dectin 3 or a binding fragment thereof is maintained in a renaturation buffer comprising arginine and denatured prior to incorporation into the liposome. Optionally, any of the methods for making liposomes described herein can further comprise storing the liposomes comprising an antifungal agent and a targeting molecule in a renaturation buffer comprising arginine. Optionally, the renaturation buffer can comprise about 0.5 to about 1.5M arginine. Optionally, the renaturation buffer can comprise about 0.1 M NaH2PO4, about 10 mM Triethanolamine, about 1 M L-Arginine, about 100 mM NaCl, about 5 mM EDTA and 5 mM BME, at pH 7.2.

In some methods, the targeting molecule is conjugated to a lipid. The lipid conjugated to the targeting molecule can be a pegylated or a non-pegylated lipid. Examples of lipids that can be conjugated to the targeting molecule and examples of antifungal agents that can be incorporated into targeted liposomes are set forth above. In some examples, the targeting molecule incorporated into the outer surface of each liposome is a C-type lectin receptor, for example, Dectin-1, Dectin-2, Dectin-3 or a fragment thereof, a chitin binding protein, an exopolysaccharid binding protein or a fragment thereof, or an antibody.

Pluralities of targeted liposomes made by the methods described can produce pluralities comprising any number of liposomes, for example, from about two to about 100,000,000 liposomes. It is understood that during the process of making targeted liposomes there may be some liposomes that do not encapsulate the antifungal agent or liposomes that do not have a targeting molecule incorporated into the outer surface of the liposomes. Therefore, provided herein are pluralities of targeted liposomes wherein at least 70%, 80%, 90%, 95%, 99% 99.5%, or 99.9% of the liposomes have an encapsulated antifungal agent and a targeting molecule that binds an antigen on a fungal cell, wherein the targeting molecule is incorporated into the outer surface of the liposomes.

### Detection of Fungal Infection

Also provided herein are liposomes comprising a targeting molecule that binds a target fungal cell antigen and a signal-generating molecule, wherein the targeting molecule is incorporated into the outer surface of the liposome, and the signal-generating molecule generates a detectable signal when the targeting molecule binds the target fungal cell antigen. In some examples, the signal-generating molecule is linked to or attached to the targeting molecule. In other examples, the signal-generating molecule is incorporated into or attached to the outer surface of the liposome. These liposomes can be used to detect the presence of fungi or a fungal infection, in vivo, ex vivo or in vitro. A fungal cell or one or more fungal cells, i.e., a population of fungal cells, can be detected. The detectable signal can be directly or indirectly detected. For example, the signal-generating molecule can be a fluorescent dye, label or probe that is directly detected (for example, rhodamine, fluorescein, green fluorescent protein, acridine orange, etc.).

In some examples, the targeting molecule is linked to a molecule that can be directly detected in vivo using imaging techniques, including, but not limited to magnetic resonance imaging, radiography, position emission tomography (PET), computed tomography (CT) scan, to name a few. Examples of molecules that can be used to detect a fungal infection using in vivo imaging include, but are not limited to, a metalloprotein, ferritin, transferrin, aquaporin, and a chemical exchange saturation transfer (CEST) report, to name a few. See, for example, Silindir et al. “Liposomes and their applications in molecular imaging,” J. Drug Target 20(5): 401-415 (2012); and Mukherjee et al. “Biomolecular MRI Reporters: evolution of new mechanisms,” Prog. Nucl. Magn Reson. Spectrosc. 102-103: 32-42 (2017)). In another example, the targeting molecule is linked to a primary antibody or a fragment thereof (for example, an Fc fragment of an antibody) that can be indirectly detected using a secondary antibody. In some examples, the target fungal antigen is on a fungal cell. In other examples, the target fungal antigen, for example, a fungal cell wall component, is released from the fungal cell into a biological sample.

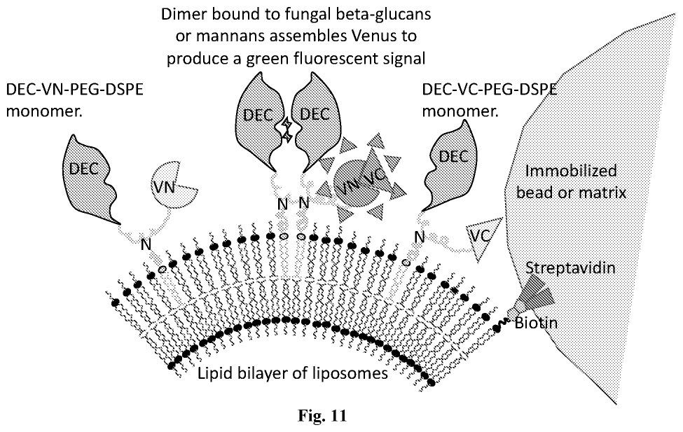

In some examples, the liposome itself generates a signal when bound to a fungal cell antigen. This signal can be generated in one or more steps after liposome binding. Examples of a one-step signaling system are shown in FIGS. 11 and 28A, where the liposomes may or may not be optionally attached to a solid support. In this example, a subject or a biological sample is contacted with liposomes that comprise a fusion protein comprising soluble Dectin (targeting molecule) linked to the C-terminal fragment of a fluorescent protein and liposomes comprising soluble Dectin (targeting molecule) linked to an N-terminal fragment of a fluorescent protein. Examples of fluorescent proteins include, but are not limited to, yellow fluorescent protein (YFP, for example, Venus), green fluorescent protein (GFP), and red fluorescent protein (RFP) as well as derivatives, for example, mutant derivatives, of these proteins. See, for example, Chudakov et al. “Fluorescent Proteins and Their Applications in Imaging Living Cells and Tissues,” Physiological Reviews 90(3): 1103-1163 (2010); and Specht et al., “A Critical and Comparative Review of Fluorescent Tools for Live-Cell Imaging,” Annual Review of Physiology 79: 93-117 (2017))

In some examples, the targeting molecule, for example, soluble Dectin-1, Dectin-2, Dectin-3 or a fragment thereof, forms dimers on the surface of a liposome upon binding beta-glucans or mannans on a fungal cell. When liposomes presenting on their surfaces both a first targeting molecule linked to an N-terminal fragment of a fluorescent protein and a second targeting molecule linked to an C-terminal fragment of a fluorescent protein is contacted with a fungal infected subject (for example, the eye, ear, throat, vagina, nasal passage, skin or nail of a subject, to name a few) or a sample from the subject (for example, urine, blood, serum, tears, speutum, lung lavage, tissue scraping or homogenate), the soluble Dectin monomers incorporated into the surface of the liposome will form dimers upon binding to beta-glucans or mannans present on fungal cells or derived from fungal cells. The first and second targeting molecules can be the same or different.

Dimer formation will affect the interaction between the C-terminal fragment of a fluorescent protein and the N-terminal fragment of the fluorescent protein, i.e., complementation (bimolecular fluorescence complementation (BiFC)), such that the signal generated from this interaction can be detected via fluorescence, for example, by a hand held fluorescent light, a fluorescent microscope, a fluorescent microtiter plate reader, a fluorescent flow cell analyzer, or other fluorescent detection instrument. Schematic models of Dectin-coated liposome that would produce a fluorescent signal when binding a fungal glucan or mannan or fungus itself are shown in FIGS. 11 and 28A. This construct enables a one-step assay, in which liposome binding to fungal polysaccharides and subsequent signal production both occur in the same, single clinical step, without washing steps or addition of additional reagents. See, for example, Kilpatrick et al. “A G Protein-Coupled Receptor Dimer Imaging Assay Reveals Selectively Modified Pharmacology of Neuropeptide Y Y1/Y5 Receptor Heterodimers,” Mol. Pharm. 87: 718-732 (2015)).

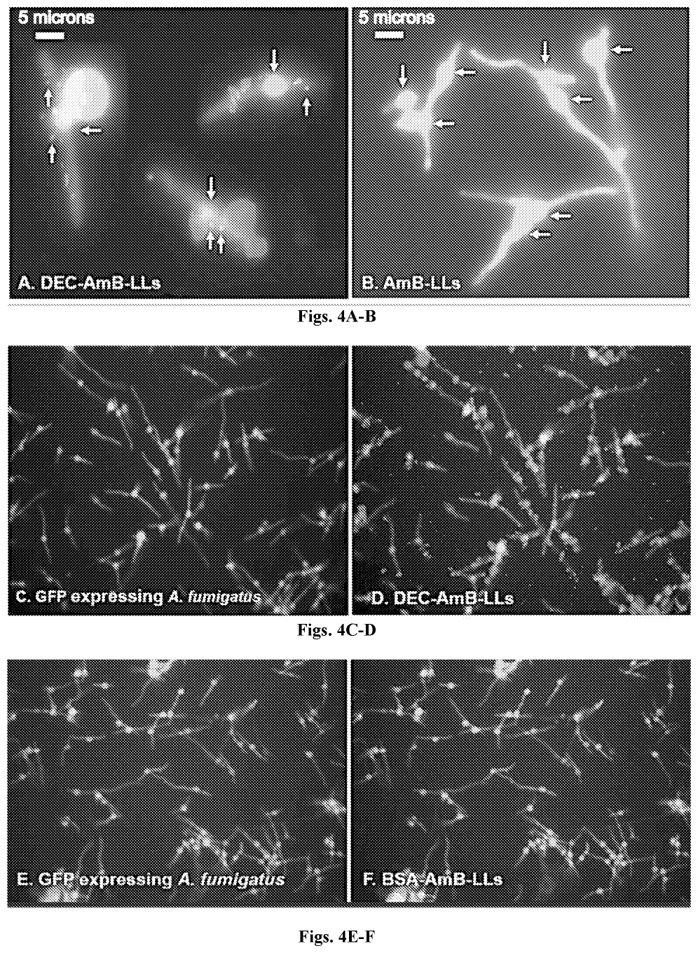

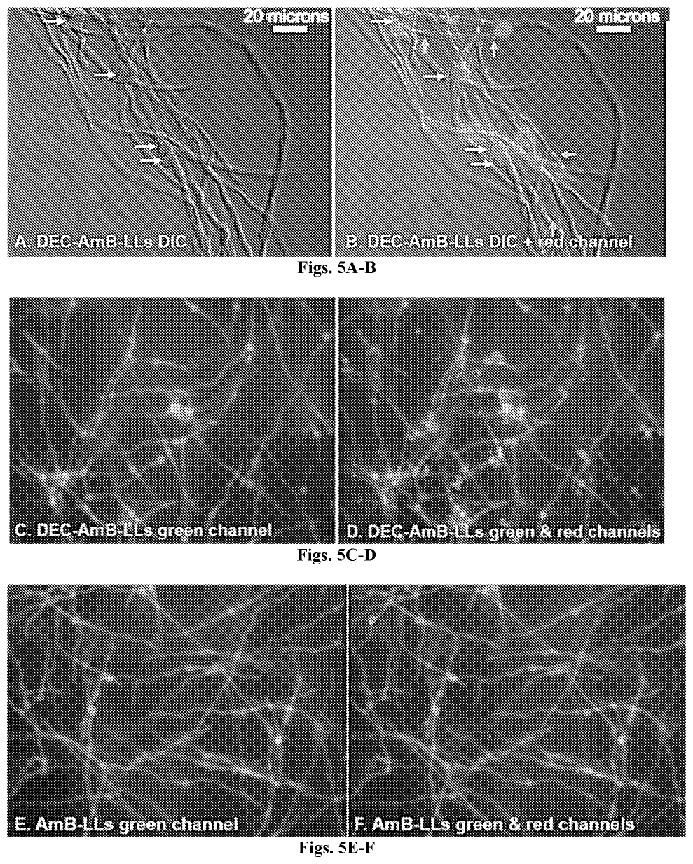

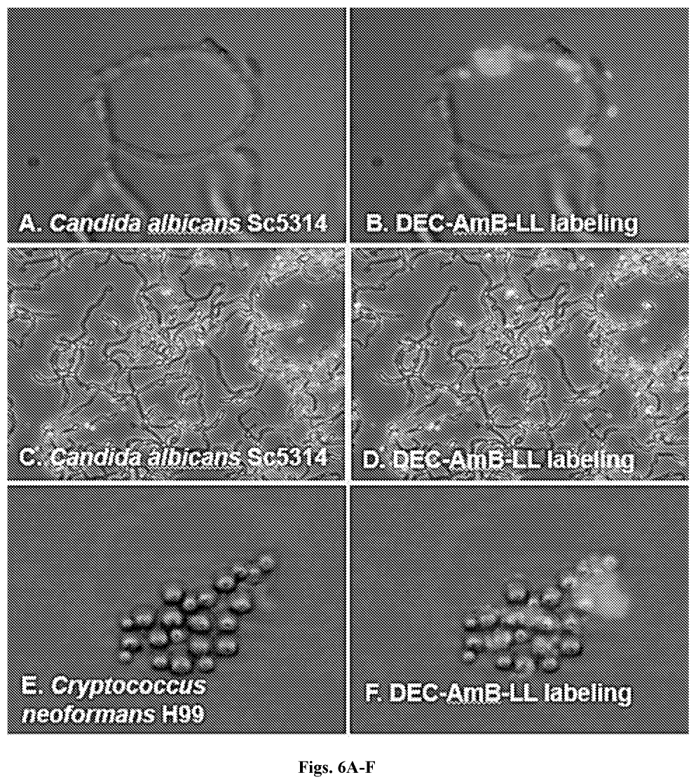

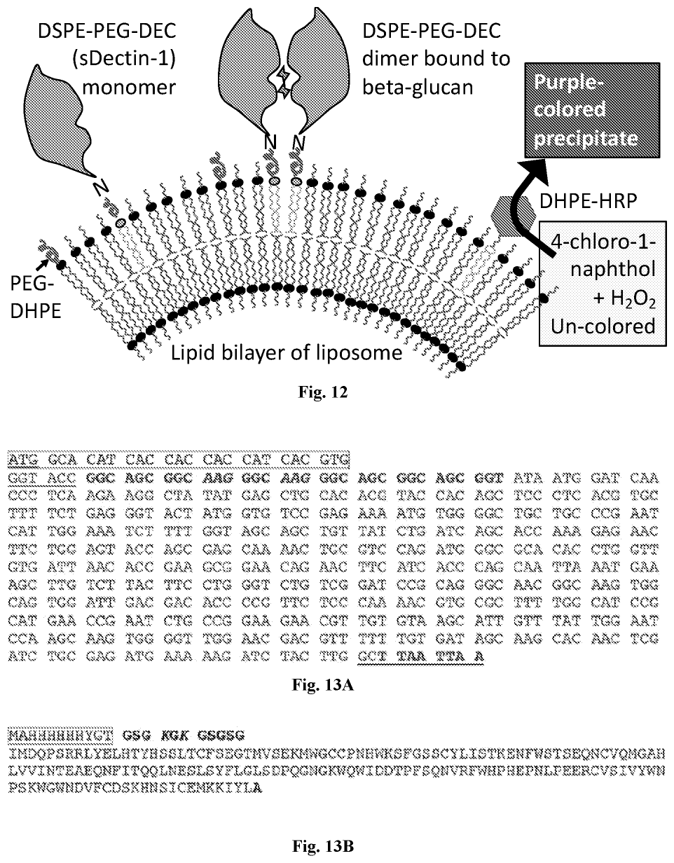

Exemplary constructs enabling two-step and three-step diagnostic assays are illustrated with rhodamine fluorescent liposomes in FIG. 3 and horseradish peroxidase linked liposomes in FIG. 12. For both diagnostic assays, the excess unbound diagnostic liposomes are washed out in a second step. The fluorescence of the remaining bound rhodamine-labeled liposomes (FIG. 3) can be assayed directly, as in FIGS. 4, 5, and 6. The HRP-coated liposomes (FIG. 12) require a third step in which the substrates for HRP enzyme activity, 4-chloro-1-naphthol and peroxide, are added and incubated for about 10 to about 30 min. The product of the enzyme is a purple precipitate that can be assayed spectrophotometrically or in a microscope. This signal is a measure of the amount of bound liposome, and hence, the amount of fungal polysaccharide. Other signal generating enzymes could be used in place of HRP, including but not limited to, luciferase (for light emission), beta-glucuronidase, and beta-galactosidase (for color production) and protamine (for MRI signal). Multi-step liposomal detection systems for fungal cells and fungal cell components could be constructed using any fungal cell antigen targeting molecule (for example, an antibody or a fragment thereof, an aptamer, a small molecule etc.), and do not require Dectin dimerization during binding.

Also provided herein is a liposome comprising a targeting molecule that binds a target antigen on a fungal cell, wherein the targeting molecule is incorporated into the outer surface of the liposome, and wherein the targeting molecule is linked or fused to a C-terminal or an N-terminal fragment of a fluorescent protein. Therefore, the liposome comprises a fusion protein comprising a targeting molecule that binds to a fungal cell antigen and the C-terminal or the N-terminal of a fluorescent protein. Pluralities of these liposomes are also provided. In some examples, the plurality includes a first subset of liposomes comprising a targeting molecule linked to an N-terminal fragment of a fluorescent protein and a second subset of liposomes comprising a targeting molecule linked to a C-terminal fragment of a fluorescent protein.

In some examples, the targeting molecule, for example, soluble Dectin-1, Dectin-2 or fragments thereof, form dimers upon binding beta-glucans or mannans on a fungal cells. When liposomes presenting on their surfaces a first targeting molecule linked to an N-terminal fragment of a fluorescent protein and a second targeting molecule linked to an C-terminal fragment of a fluorescent protein is contacted with a fungal infected subject (for example, the eye, ear, throat, vagina, nasal passage, skin or nail of a subject, to name a few) or a sample from the subject (for example, urine, blood, serum, tears, sputum, lung lavage, tissue scraping or homogenate), the soluble Dectin-1, Dectin-2, or Dectin-3 monomers incorporated into the surface of the liposomes will form dimers upon binding to beta-glucans or mannans on any fungal cell present in the subject or in a sample from the subject as well as any soluble beta-glucans or mannans released from these fungi. Dimer formation will affect the interaction between the C-terminal fragment of a fluorescent protein and the N-terminal fragment of the fluorescent protein, i.e., complementation (e.g., bimolecular fluorescence complementation (BiFC)), such that the signal generated from this interaction can be detected via fluorescence, for example, by using an a fluorescent microscope, a fluorescent microtiter plate reader, a fluorescent flow cell analyzer, or other fluorescent detection instrument. See, for example, Kilpatrick et al. “A G Protein-Coupled Receptor Dimer Imaging Assay Reveals Selectively Modified Pharmacology of Neuropeptide Y Y1/Y5 Receptor Heterodimers,” Mol. Pharm. 87: 718-732 (2015)). Schematic models of Dectin-coated liposomes that would produce a fluorescent signal when binding a fungal glucan or mannan or fungus itself are shown in FIGS. 11 and 28A. This construct enables a one-step assay, in which liposome binding to fungal polysaccharides and subsequent signal production both occur in the same single clinical step, without washing steps or addition of additional reagents. See, for example, Kilpatrick et al. “A G Protein-Coupled Receptor Dimer Imaging Assay Reveals Selectively Modified Pharmacology of Neuropeptide Y Y1/Y5 Receptor Heterodimers,” Mol. Pharm. 87: 718-732 (2015)).

Further provided herein is a fusion polypeptide comprising a targeting molecule that binds a target fungal cell antigen and the N-terminal or the C-terminal portion of a fluorescent polypeptide. These fusion polypeptides can be used to detect a fungal infection. In some examples, the targeting molecule is a C-type lectin receptor, an antibody, a fungal cell wall binding protein, a chitin binding protein or a fragment thereof. In some examples, the targeting molecule is Dectin-1, Dectin-2, Dectin-3 or a fragment thereof. In some examples, the fluorescent protein is yellow fluorescent protein or a derivative thereof.

Provided herein is a method for detecting a fungal infection in a subject or a sample from the subject comprising (a) contacting the subject or a sample from the subject with a plurality of liposomes, wherein each liposome in the plurality comprise a targeting molecule that binds a target fungal cell antigen and a signal-generating molecule, wherein the targeting molecule is incorporated into the outer surface of the liposome, and the signal-generating molecule generates a detectable signal when the targeting molecule binds the target fungal cell antigen; and b) detecting a signal, wherein a signal indicates the presence of a fungal infection. This method can be used to detect a fungal infection in vivo, ex vivo or in vitro. In some examples, the fungal cell antigen is a fungal cell antigen on a cell. In other examples, the fungal cell antigen is a soluble fungal cell antigen, for example, beta-glucans or mannins in a biological sample. In some examples, the targeting molecule is linked to the signal-generating molecule. In other examples, the signal-generating molecule is incorporated into or attached to the outer surface of the liposome. In some examples, the targeting molecule is linked to a signal generating enzyme, for example, HRP, luciferase, beta-glucuronidase, and beta-galactosidease. In other examples, the targeting molecule is linked to a fluorescent protein, for example, rhodamine, GFP, YFP, RFP, etc. Fragments of fluorescent proteins, for example, the N-terminal or the C-terminal of any fluorescent protein can be linked to the targeting molecule. In other examples, the targeting molecule is linked to antibody, or a fragment thereof.

Provided herein is a method for detecting a fungal infection in a subject or a sample from the subject comprising (a) contacting the subject or a sample from the subject with a plurality of liposomes, wherein each liposome in the plurality comprises a targeting molecule linked to an N-terminal fragment of a fluorescent protein and a targeting molecule linked to an C-terminal fragment of a fluorescent protein, wherein the targeting molecule binds a target antigen on a fungal cell, and (b) detecting a fluorescence signal generated by the interaction between the N-terminal and the C-terminal fragment of the fluorescent protein, wherein a signal indicates the presence of a fungal infection. In some methods, the fluorescence signal is detected using an ultraviolet light or fluorescent microscope or instruments for quantitative fluorescence detection of bimolecular fluorescence complementation (i.e., BiFC analysis).

In some methods for detecting a fungal infection, each liposome in the plurality comprises a) at least about 500 targeting molecules linked to the N-terminal fragment of a fluorescent protein; and b) at least about 500 targeting molecules linked to the C-terminal fragment of a fluorescent protein. For example, each liposome can comprise at least about 250, 500, 100, 1500, 2000 or 2500 targeting molecules linked to the N-terminal fragment of a fluorescent protein and at least about 250, 500, 100, 1500, 2000 or 2500 targeting molecules linked to the C-terminal fragment of a fluorescent protein.

Further provided is a method of detecting a fungal infection in subject or a sample from the subject comprising: a) contacting the subject or a sample from the subject with a first plurality of fusion polypeptides comprising a targeting molecule that binds a target fungal cell antigen and the N-terminal portion of a fluorescent polypeptide and second plurality of fusion polypeptides comprising a targeting molecule that binds a target fungal cell antigen and the C-terminal portion of a fluorescent polypeptide; and b) detecting a fluorescence signal generated by the interaction between the N-terminal and the C-terminal fragment of the fluorescent protein, wherein a signal indicates the presence of a fungal infection.

In some examples, the plurality of liposomes is immobilized on a solid support. Non-limiting examples of solid support materials include glass, modified or functionalized glass, plastics including acrylics, polystyrene and copolymers of styrene and other materials, polypropylene, polyethylene, polybutylene, polyurethanes, or TeflonJ, nylon, nitrocellulose, polysaccharides, resins, silica or silica-based materials including silicon and modified silicon, carbon, metals, inorganic glasses and plastics. The size and shape of the solid support can vary. A solid support can be planar, a solid support can be a well, or alternatively, a solid support can be a bead or a slide. In some embodiments, a solid support is a well of a multiwell plate. In other examples, the solid support can be a magnetic bead, an agarose-based resin or an agarose bead. In other examples, the solid support comprises non-agarose chromatography media, monoliths or nanoparticles. For example, the chromatography media can be, e.g., methacrylate, cellulose, or glass. In other examples, the nanoparticles are gold nanoparticles or magnetic nanoparticles.

As used throughout, by subject is meant an individual. The subject can be an adult subject or a pediatric subject. Pediatric subjects include subjects ranging in age from birth to eighteen years of age. Thus, pediatric subjects of less than about 10 years of age, five years of age, two years of age, one year of age, six months of age, three months of age, one month of age, one week of age or one day of age are also included as subjects. Preferably, the subject is an animal, for example, a mammal such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

As used herein, a biological sample is a sample derived from a subject and includes, but is not limited to, any cell, tissue or biological fluid. The sample can be, but is not limited to, blood, plasma, serum, sputum, urine, saliva, bronchoalveolar lavage fluids, biopsy (e.g. tissue or cells isolated from organ tissue, for example, from lung, liver kidney, skin etc.), vaginal secretion, nasal secretion, skin, gastric secretion or bone marrow specimens.

The subject can also be a plant or a seed from a plant. A biological sample can also be derived from plant, but is not limited to, any cell, tissue or plant exudate. The sample can be, but is not limited to the surface of leaves, stems, roots, petals, sepals, stamens, carpels, and seeds or crushed samples or extracts of the same.

### Methods for Treating or Preventing a Fungal Infection

Also provided are methods for treating or preventing a fungal infection in a subject. The methods comprise administering to the subject having a fungal infection or at risk of developing a fungal infection an effective amount of a plurality of any of the liposomes described herein, wherein each liposome in the plurality comprises an antifungal agent and a targeting molecule that binds a target antigen on a fungal cell, wherein the targeting molecule is incorporated into the outer surface of the liposome and the antifungal agent is encapsulated in the liposome. Any of the liposomes or pluralities of liposomes provided herein can be in a pharmaceutical composition.

The methods can be used to treat or prevent a fungal infection in any animal, for example, a human. Examples of human fungal infections include, but are not limited to, Alternaria alternata, Aspergillus species such as A. fumigatus, Blastomyces species such as B. dermatitides, Candida species such as C. albicans, C. glabrata, C. krusei, C. auris, Coccidioides species such as C. immitis and C. posadasii, Cryptococcus species such as C. gattii and C. neoformans, Histoplasma species such as H. capsulatum, Pneumocystis species such as P. jirovecii, Sporothrix species such as S. schenckii, Talaromyces marneffei (formerly Penicillium marneffei), and Trichophyton rubrum.

Throughout, treat, treating, and treatment refer to a method of reducing or delaying one or more effects or symptoms of a fungal infection. The subject can be diagnosed with a fungal infection. Treatment can also refer to a method of reducing the underlying pathology rather than just the symptoms. The effect of the administration to the subject can have the effect of, but is not limited to, reducing one or more symptoms of the disease, a reduction in the severity of the disease, the complete ablation of the disease, or a delay in the onset or worsening of one or more symptoms. For example, a disclosed method is considered to be a treatment if there is about a 10% reduction in one or more symptoms of the disease in a subject when compared to the subject prior to treatment or when compared to a control subject or control value. Thus, the reduction can be about a 10, 20, 30, 40, 50, 60, 70, 80, 90, 100%, or any amount of reduction in between.

As used herein, by prevent, preventing, or prevention is meant a method of precluding, delaying, averting, obviating, forestalling, stopping, or hindering the onset, incidence, severity, or recurrence of a disease or disorder. For example, the disclosed method is considered to be a prevention if there is a reduction or delay in onset, incidence, severity, or recurrence of fungal infection in a subject susceptible to a fungal infection or recurrence of a fungal infection compared to control subjects susceptible to a fungal infection or recurrence of a fungal infection that did not receive treatment. The reduction or delay in onset, incidence, severity, or recurrence of a fungal infection can be about a 10, 20, 30, 40, 50, 60, 70, 80, 90, 100%, or any amount of reduction in between.

In some methods, the subject is immunocompromised. For example, the subject can be a subject that has undergone a stem cell, organ, tissue or bone marrow transplant, a subject that has cancer, a subject receiving cancer therapy (for example, chemotherapy, immunotherapy or radiotherapy), a subject taking corticosteroids, a subject infected with HIV or having acquired immunodeficiency syndrome, a subject that has hepatitis, a subject with a B-cell defect or a subject with a T-cell defect, to name a few.

In some methods, the subject has one or more disorders that affect lung function in the subject, for example, pulmonary fibrosis, pneumonia, asthma, chronic obstructive pulmonary disease (COPD), cystic fibrosis, tuberculosis, emphysema or sarcoidosis.

The methods provided herein optionally include selecting a subject with a fungal infection or at risk of a fungal infection. One of skill in the art knows how to diagnose a subject with a fungal infection. For example, a medical examination can be performed. One or more of the following tests can be also used: microscopic examination of clinical samples, histopathology, culture, and serology. Molecular diagnostics and antigen detection in clinical samples can also be used (See, for example, Kozel and Wickes “Fungal Diagnostics,” Cold Spring Harb. Perspect. Med. 4(4): a019299 (2014)).

The methods provided herein optionally further include administering an effective amount of a second therapeutic agent or therapy to the subject. The second therapeutic agent or therapy can be administered to the subject prior to, simultaneously with, or subsequent to administration of the plurality of liposomes. In some methods, the second therapeutic therapy is surgery. In some methods, the second therapeutic agent is a second antifungal agent. The antifungal agent can be any of the polyene antifungals, azole antifungals, imidazoles, triazoles or echinocandins described above.

### Pharmaceutical Compositions

The term effective amount, as used throughout, is defined as any amount necessary to produce a desired physiologic response, for example, treating or preventing a fungal infection. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, unwanted cell death, and the like. Generally, the dosage will vary with the type of inhibitor, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. Dosages can vary and can be administered in one dose or multiple doses administered daily or at extended intervals.

Any of the liposomes described herein can be provided in a composition, for example, a pharmaceutical composition. The composition can include one or more liposomes disclosed herein. Optionally, the composition comprising one or more liposomes is in a kit. Pharmaceutical compositions include, for example, a pharmaceutical composition comprising a therapeutically effective amount of any of the liposomes described herein and a pharmaceutical carrier. The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water.

Pharmaceutical compositions comprising any of the liposomes described herein can be prepared according to standard techniques and further comprise a pharmaceutically acceptable carrier. Generally, normal saline will be employed as the pharmaceutically acceptable carrier. Other suitable carriers include, e.g., water, buffered water or saline, 0.4% saline, 0.3% glycine, dextrose, and the like, including glycoproteins for enhanced stability, such as albumin, lipoprotein, and globulin. These compositions are usually sterile. The pharmaceutical compositions can also contain a pharmaceutically acceptable excipient. Such excipients include any pharmaceutical agent that does not itself induce an immune response harmful to the individual receiving the composition, and which may be administered without undue toxicity. Pharmaceutically acceptable excipients include, but are not limited to, liquids such as water, saline, glycerol, sugars and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. The preparation of pharmaceutically acceptable carriers, excipients and formulations containing these materials is described in, e.g., Remington: The Science and Practice of Pharmacy, 22nd edition, Loyd V. Allen et al, editors, Pharmaceutical Press (2012).

Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, and calcium chloride. Additionally, the liposome suspension may include lipid-protective agents which protect lipids against free-radical and lipid-peroxidative damages on storage. Lipophilic free-radical quenchers, such as alphatocopherol and water-soluble iron-specific chelators, such as ferrioxamine, are suitable.

The concentration of the liposomes in the pharmaceutical formulations can vary widely, i.e., from less than about 0.05%, usually at or at least about 2-5% to as much as 10 to 30% by weight and will be selected primarily by fluid volumes, viscosities, in accordance with the particular mode of administration selected. Or the liposomes may be dried or lyophilized and resuspended to a desired concentration in water or buffers at time of use. The amount of liposomes or the amount of active agent in the liposome administered depends upon the particular label used, the disease state being diagnosed and the judgment of the clinician but is generally between about 0.01 and about 150 mg per kilogram of body weight, preferably between about 0.1 and about 20 mg/kg of body weight, about 0.1 to about 10 mg/kg of body weight or about 0.1 to about 5 mg/kg of body weight, which may be administered in a single dose or in the form of individual doses, such as from 1 to 4 times per day. Administration can be performed for 1, 2, 3, 4, 5, 6, 7, 8, 9, 10, 11, 12, 13,14, 15, 16, 17, 18, 19, 20 or more days. One of skill in the art would adjust the dosage as described below based on specific characteristics of the agent and the subject receiving it.

The compositions disclosed herein are administered in a number of ways depending on whether local or systemic treatment is desired, and on the area to be treated. The compositions are administered via any of several routes of administration, including orally, intranasally, via inhalation, via nebulizer, parenterally, intravenously, intraperitoneally, intracranially, intraspinally, intrathecally, intraventricularly, intramuscularly, subcutaneously, intracavity or transdermally. Pharmaceutical compositions can also be delivered locally to the area in need of treatment, for example by topical application or local injection. The pharmaceutical compositions can also be delivered via pump or at a surgical site. Effective doses for any of the administration methods described herein can be extrapolated from dose-response curves derived from in vitro or animal model test systems.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

The following examples are provided by way of illustration only and not by way of limitation. Those of skill in the art will readily recognize a variety of non-critical parameters that could be changed or modified to yield essentially the same or similar results.

### Example 1

### Dectin-1

### Fungal Growth

Aspergillus fumigatus strain A1163 was transformed with plasmid pBV126 described in Kang et al. (“A dual selection based, targeted gene replacement tool for Magnaporthe grisea and Fusarium oxysporum. Fungal Genet Biol 42:483-492 (2005)) carrying green fluorescent protein EGFP under the control of Magnaporthe oryzae ribosomal protein 27 promoter was used to monitor fungal cells in some experiments to yield the strain AEK012. A. fumigatus spores were grown on plates in Vogel's Minimal Media (VMM, 1% glucose, 1.5% Agar) for 7 days and conidia collected in PBS+0.1% Tween. For fluorescent liposome localization and for growth inhibition and killing assays 20,000 and 4,500 AEK012 conidia were plated on 24 well and 96 well poly-L-lysine coating plates, respectively, in VMM, 1% glucose, 0.5% BSA at 37° C. for various time periods ranging from 8 hr to 4 days (Momany 2001 Chapter 7. Using Microscopy to explore the duplication cycle, p 119-125. In Talbot N J (ed), Molecular and cellular biology of filamentous fungi: a practical approach Oxford University Press, University of Exeter, Exeter, UK; and Sasaki et al. (Heterochromatin controls gammaH2A localization in Neurospora crassa. Eukaryot Cell 13:990-1000 (2014)). Candida albicans Sc5314 and Cryptococcus neoformans H99 were pre-grown in Potato Dextrose liquid media for overnight. The cells were then washed 3 times with sterile water and resuspended in Roswell Park Memorial institute (RPMI) media and grown on poly-L-lysine coated plates at 37° C. for 10 hours. All fungal cell growth was carried out in a BSL2 laboratory. For fluorescent imaging of liposomal stained fungi, all three fungi were washed 3 times with PBS, fixed in 4% formaldehyde in PBS for 15 to 60 mins, washed once and stored at 4° C. in PBS.

### Production of Soluble Dectin-1

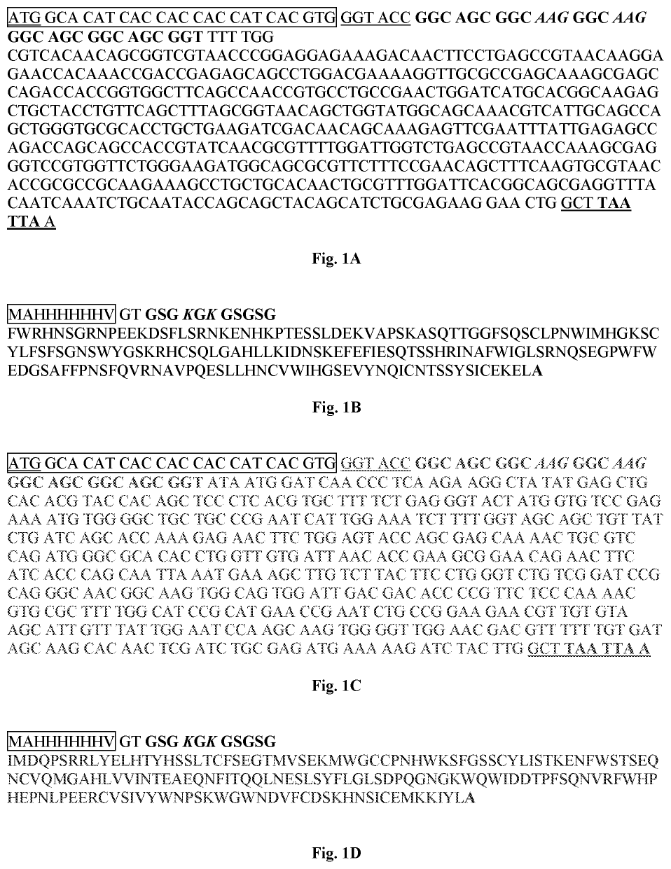

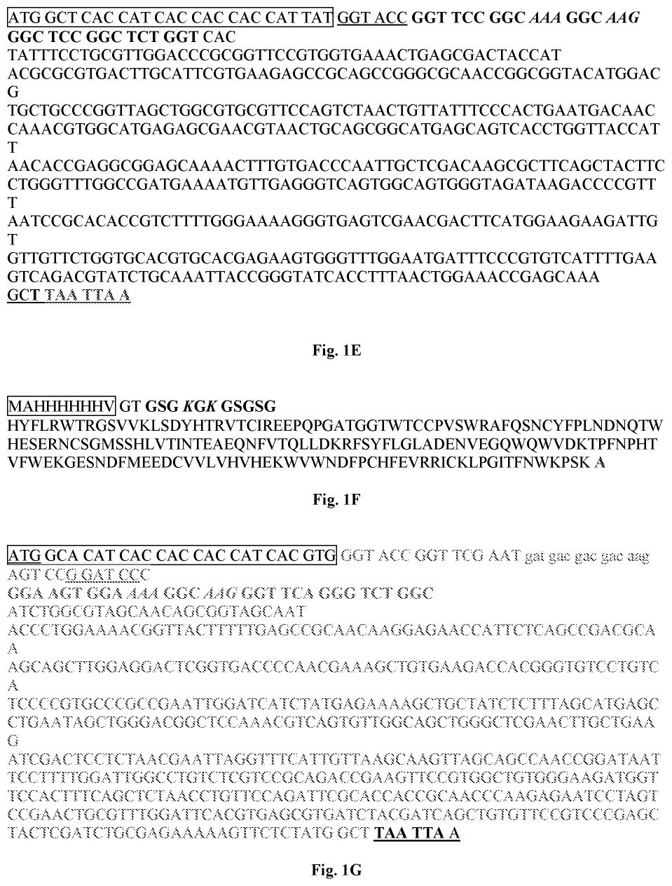

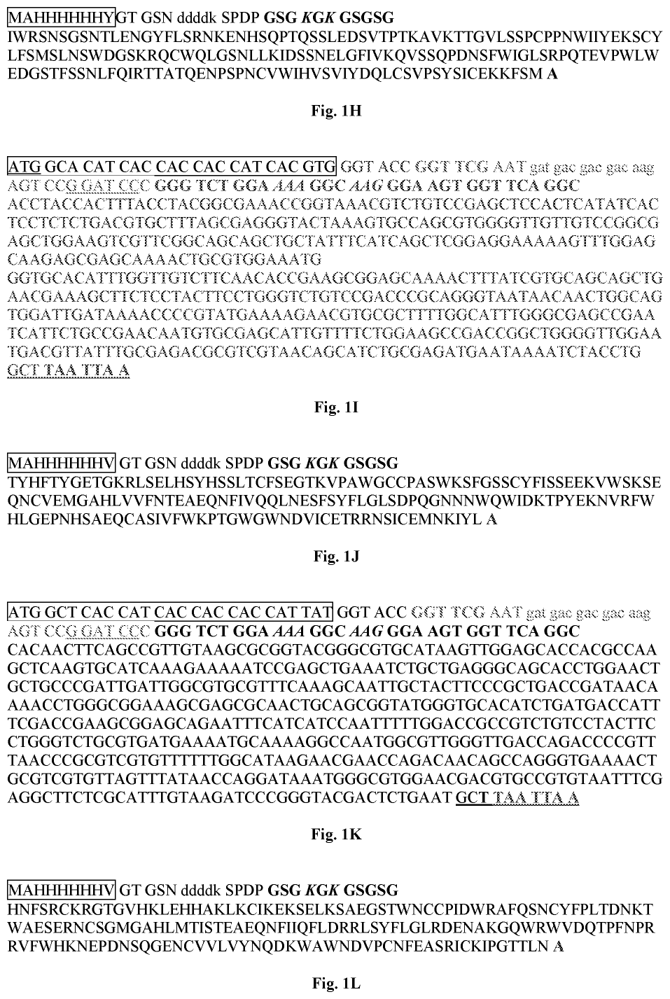

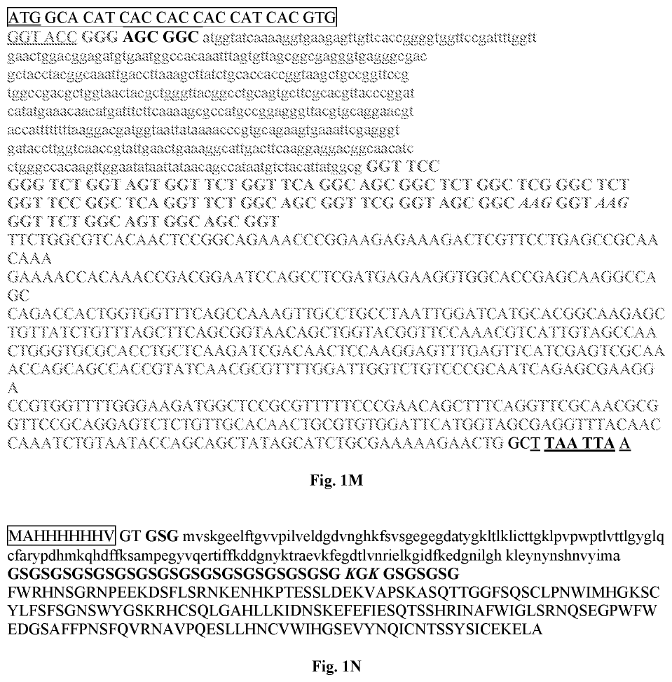

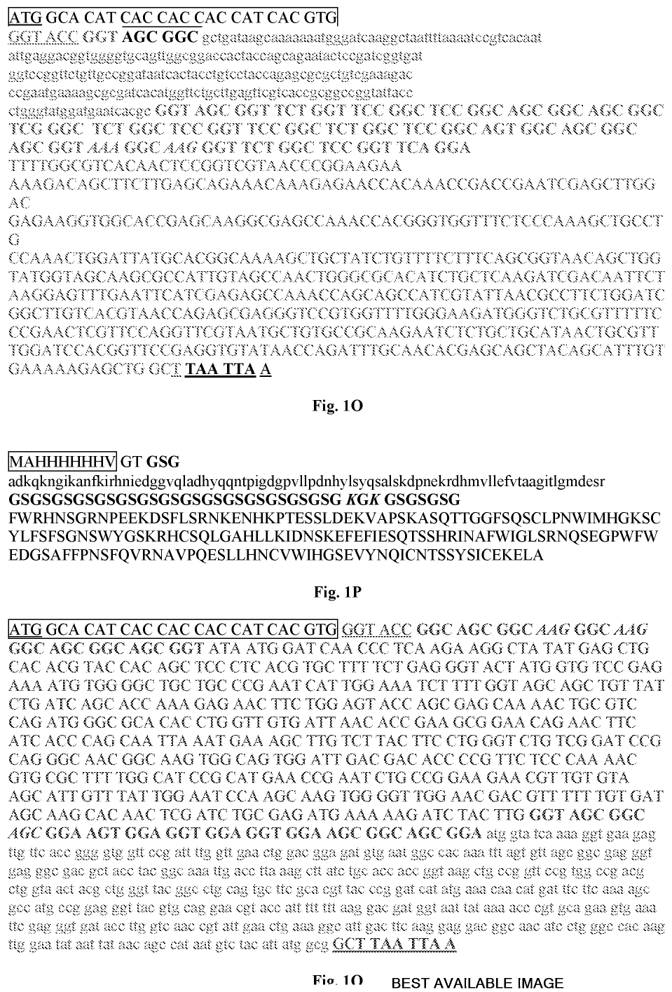

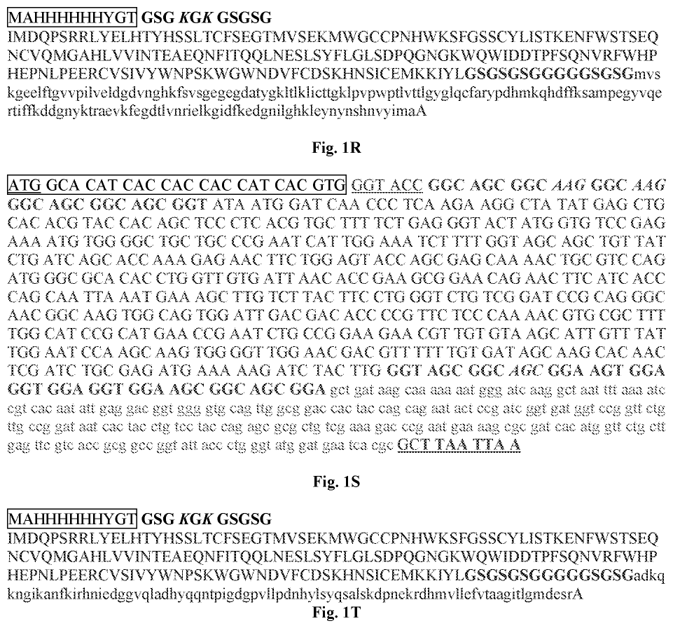

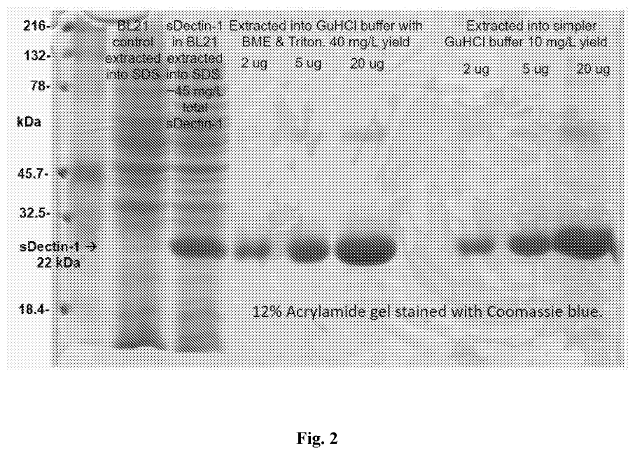

The sequence of an exemplary codon-optimized E. coli expression construct with Ms-sDectin-1 cloned into pET-45B (GenScript) is shown in FIG. 1). This construct encoded a slightly modified 198 a.a. long sDectin-1 protein containing a vector specified N-terminal (His)₆ affinity tag, a flexible spacer, two lysine residues, another flexible spacer followed by the C-terminal 176 a.a. murine sDectin-1 domain. Starting with 1 L of bacterial culture (BL21 strain) grown overnight in Luria broth without IPTG induction, approximately 45 mg/L of 22 kDa sDectin-1 were obtained (FIG. 2). The sDectin-1 was extracted from cell pellets in pH=8.0, 6 M guanidine hydrochloride (GuHCl, Fisher BioReagents BP178), 0.1 M Na2HPO4/NaH2PO4, 10 mM Triethanolamine, 100 mM NaCl, 5 mM BME, 0.1% Triton-X100. sDectin-1 was bound to a nickel affinity resin (QiaGen, #30210) in this buffer, washed in this buffer adjusted to pH 6.3, and eluted in this buffer adjusted to ph 4.5. The pH of the eluted protein was immediately neutralized to pH 7.2 with 1 M pH 10.0 M triethanolamine for long term storage. Forty mg of greater than 95% pure protein was recovered (FIG. 2). Samples of sDectin-1 at 6 ug/uL in this same GuHCl buffer with fresh 5 mM BME added were further adjusted to pH 8.3 with triethanolamine and reacted a 4-molar excess of lipid carrier reagent DSPE-PEG-3400-NHS (Nanosoft polymers, 1544-3400) for 1 hr at 23° C. to make DSPE-PEG-DEC. Gel exclusion chromatography on Bio-Gel P-6 acrylamide resin (Bio-Rad #150-0740) in renaturation and storage buffer RN #5 (0.1 M NaH2PO4, 10 mM Triethanolamine, pH 7.2, 1 M L-Arginine, 100 mM NaCl, 5 mM EDTA, 5 mM BME) was used to remove unincorporated DSPE-PEG and GuHCl. The composition of RN #5 was determined empirically by testing a large number of mild denaturing and crowding buffers, most of which resulted in the modified Dectin-1 protein falling out of solution after a few days. The protein remains in solution indefinitely, when stored in RN #5 and if freshly reduced with BME it may be readily renatured into an active carbohydrate binding form, when diluted from RN #5 into normal biological buffers. DSPE-PEG-BSA was prepared from bovine serum albumin BSA (Sigma, A-8022) by the same protocol.

Remote Loading of Amphotericin B, sDectin-1, BSA, and Rhodamine into Liposomes

Sterile pegylated liposomes were obtained from FormuMax Sci. Inc. (DSPC:CHOL:mPEG2000-DSPE, 50:45:5 mole %, 100 nm diameter, 60 umole/mL lipid in liposomal suspension, ˜4×10¹² liposomes/mL, #F10203A). Commercial AmBisomes (amphotericin b liposomes for injection, Gilead, Avanti) contain approximately 11 moles percent AmB. Small batches of liposomes were remotely loaded with 11 moles percent Amphotericin B (AmB, Sigma A4888) relative to liposomal lipid to make AmBisome-like AmB-LLs were used throughout this study. For example, AmB (1.8 mg, 1.95 umoles) was dissolved in 13 uL DMSO by heating 10 to 20 min and occasional mixing at 60° C. to make an oil-like clear brown AmB solution. Two hundred and fifty uL of sterile liposomal suspension (15 umoles of liposomal lipid) was added to the AmB-oil and mixed on a rotating platform for 96 hours at 37° C. at which time most of the AmB was intercalated into the liposomes. The unincorporated AmB (0.3 umoles) remained in the oil phase as quantified by analysis at A406, while 1.65 umoles was bound in liposomes, making the liposomes 11 moles percent AmB relative to moles of lipid. In separate preparations gel exclusion chromatography on BioGel A-0.5 M agarose resin (BioRad 151-0140) and examining the excluded fractions at A406 confirmed that 11 moles percent AmB was retained in the liposomes. Larger or smaller amounts of AmB may be loaded into liposomes by starting with higher or lower amounts of AmB in the oil phase. These liposomes with approximately 11 moles percent AmB are termed AmB-LLs throughout.

The DSPE-PEG-sDectin-1 and DSPE-PEG-BSA conjugates in RN #5 buffer were integrated via their DSPE moiety into the phospholipid bilayer membrane of AmB-LLs at 1.0 and 0.33 moles percent of protein relative to moles of liposomal lipid, respectively, by 60 min incubation at 60° C. to make DEC-AmB-LLs and BSA-AmB-LLs. During this same 60° C. incubation, the red fluorescent tag, Lissamine rhodamine B-DHPE triethanolamine salt (Invitrogen, #L1392) was also incorporated at two moles percent relative to liposomal lipid (Yao et al. pHLIP(R)-mediated delivery of PEGylated liposomes to cancer cells. J Control Release 167:228-237 (2013); He et al. Immunoliposome-PCR: a generic ultrasensitive quantitative antigen detection system. J Nanobiotechnology 10

:26 (2012); and Garrett et al. Liposomes fuse with sperm cells and induce activation by delivery of impermeant agents. Biochim Biophys Acta 1417:77-88 (1999)). Gel exclusion chromatography on BioGel A-0.5 M resin confirmed that Rhodamine-DHPE and DSPE-PEG-protein insertion into liposomes were essentially quantitative at these mole ratios. DEC-AmB-LLs stored at 4° C. retained binding specificity for approximately 2 months. Storage under free conditions may extend shelf life.

### Microscopy of Liposome Binding

Formalin fixed or live fungal or animal cells were incubated with DEC-AmB-LLs, BSA-AmB-LL and AmB-LLs liposomes in liposome dilution buffer LDB (PBS pH 7.2, 0.5% BSA, 5 mM BME), and unbound liposomes washed out after 15 min to 2 hr incubation (see individual figures) in the same LDB. Images of rhodamine red fluorescent liposomes, green EGFP A. fumigatus and differential interference contrast (DIC) illuminated cells were taken under oil immersion at 63× on a Leica DM6000B automated microscope (FIG. 4A, 4B, 5A, 5B, 6A, 6B). Cells were removed from plates and spread on microscope slides. Seven Z-stack images were recorded at one micron intervals and were merged in Adobe Photoshop CC2018. Bright field and/or red and/or green fluorescent images were taken directly of cells on microtiter plates at 10×, 20× or 40×, on an Olympus IX70 Inverted microscope and an Olympus PEN E-PL7 digital camera and the bight field and/or colored layers merged in Photoshop (FIG. 4C-4F, 5C-5F, 6C-6F).

### Cell Growth and Viability Assays

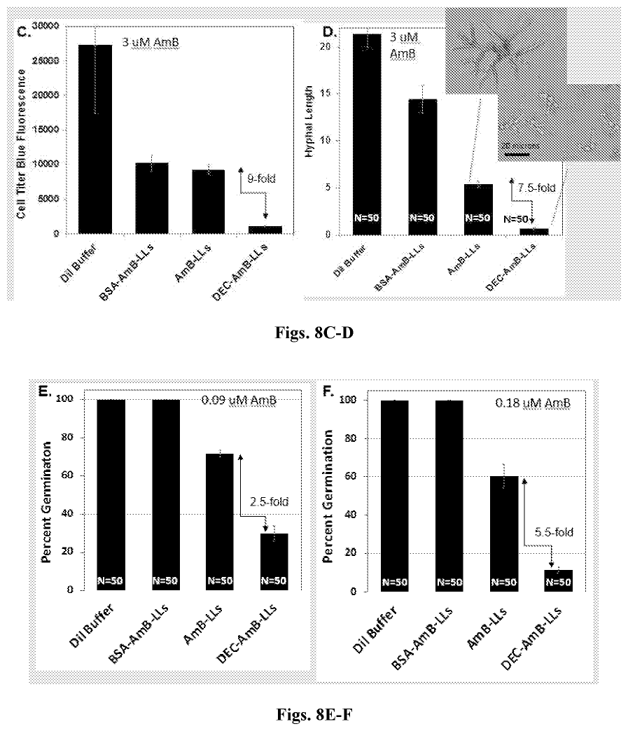

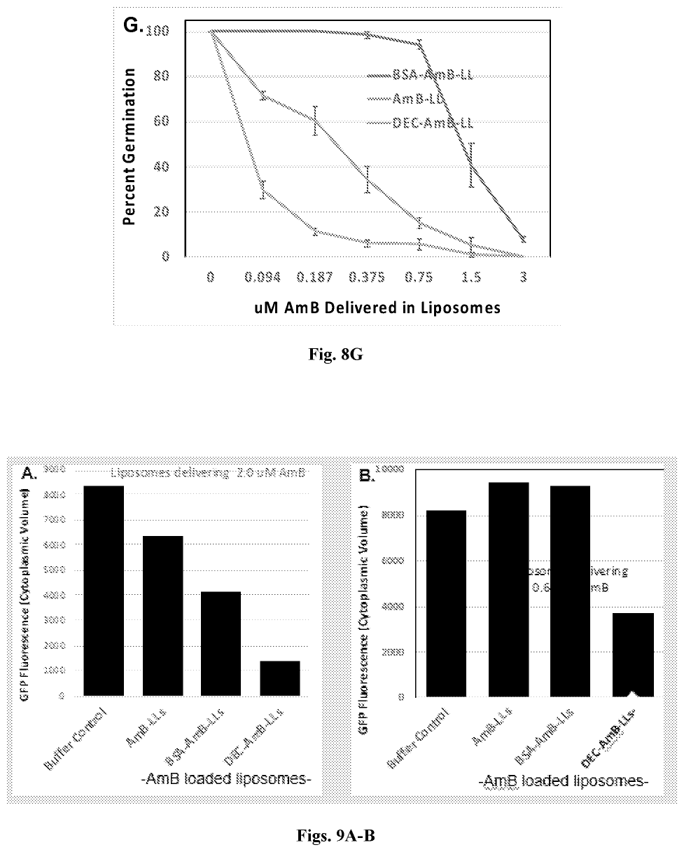

Liposomal stocks were stored at 800 uM AmB and diluted first 2 to 20-fold into liposome dilution buffer (LDB) or growth media and then further diluted 10-fold or 20-fold into growth media with cells for use at the indicated concentrations. Total dilutions typically ranged from 250-fold to 4,000-fold. CellTiter-Blue (CTB) cell viability assays were conducted as per the manufacturer's instructions (Promega, document #G8080) using 20 uL of substrate to treat 100 or 200 uL of fungal or animal cells in growth media and incubating for 4 hours at 37° C. before stopping the reaction by the addition of 50 uL of 3% SDS. Red fluorescence of esterase CTB product (Ex485/Em590) was measured in a Biotek Synergy HT microtiter plate reader. Data from six wells was averaged for each data point and standard error calculated (FIGS. 8A, 8C). Data for germination (FIGS. 8E-8F) and hyphal length (FIGS. 8B, 8D) assays were collected manually from multiple photographic images taken at 10× and/or 20×. Green fluorescence assays of EGFP expressing A. fumigatus A1163 cell viability were performed on a microtiter plate reader at Ex495/Em520 after the cells were fixed in 4% formaldehyde and PBS (FIG. 9).

### Results

Preparation of Amphotericin B Loaded sDectin-1 Coated Liposomes

Pegylated liposomes were remotely loaded with 11 moles percent AmB relative to moles of liposomal lipids to make control AmB-Loaded Liposomes, AmB-LLs, which are similar in structure and AmB concentration to commercial AmBisomes (Gilead AmBisomes). sDectin-1 (DEC, FIG. 1, FIG. 2) and Bovine serum albumin (BSA) were modified with a pegylated lipid carrier, DSPE-PEG. One mole percent DSPE-PEG-DEC was incorporated into AmB-LLs to make sDectin-1 coated DEC-AmB-LLs (FIG. 3) and 0.33 mole percent DSPE-PEG-BSA was incorporated into AmB-LLs to make BSA-Amb-LLs. This mole ratio of sDectin-1 (MW 22 kDa) and BSA (MW 65 kDa) results an equivalent ug amounts of protein coating both sets of liposomes. Because these protein coated liposomes were made from the same AmB-LLs, all three liposomal preparations contain 11 moles percent AmB relative to moles of lipid. Two moles percent of DHPE-Rhodamine were loaded into all three classes of liposome to make red fluorescent AmB-LLs, BSA-AmB-LLs, and DEC-AmB-LLs.

sDectin-1 Coated Liposomes DEC-AmB-LLs Bind Strongly to Fungal Cells

In assays performed on A. fumigatus germlings, rhodamine red fluorescent DEC-AmB-LLs bound strongly to swollen conidia and to germ tubes as shown in FIG. 4. The sDectin-1-targeted liposomes often bound in large numbers or aggregates to particular regions. While 100 nm liposomes are too small to be resolved by light microscopy, individual liposomes are visible as somewhat uniformly sized small red fluorescent dots (orange arrows, FIG. 4A). Because each liposome contains over a thousand rhodamine molecules (FIG. 3), they each fluoresce strongly enough to be visualized as single liposomes. Essentially all germlings bind DEC-AmB-LL (FIGS. 4C and 4D). Binding to un-germinated conidia was not detected (not shown). AmBisome-like AmB-LLs (FIG. 4B) and bovine serum albumin coated liposomes, BSA-AmB-LLs (FIGS. 4E & 4F) did not bind detectably to conidia or germtubes. Maximum labeling by DEC-AmB-LLs was achieved within 15 to 30 min and the strong red fluorescent signals of DEC-AmB-LLs bound to cells were maintained for weeks, when plates were stored in the dark in PBS at 4° C.

DEC-AmB-LLs also bound to swollen conidia and hyphae from more mature cells as shown in FIG. 5. Again, the sDectin-1-targeted liposomes often bound in clumps, but some fairly uniformly sized individual small red dots are visible (orange arrows, FIG. 5A), which appear to be individual fluorescent liposomes. Unlike the labeling of germ tubes, DEC-AmB-LLs were only bound to a subset of mature hyphae (FIGS. 5C and 5D) as in earlier reports on the binding of various sDectin-1 preparations. AmB-LLs did not bind detectably to mature hyphae (FIGS. 5E & 5F) nor did BSA-AmB-LLs (not shown). Finally, DEC-AmB-LLs also labeled Candida albicans hyphae and Cryptococcus neoformans H99 cells (FIG. 6). In short, Dectin-coated Amphotericin B loaded liposomes efficiently bound to fungal cells, while uncoated AmBisome-like liposomes and BSA coated liposomes did not.

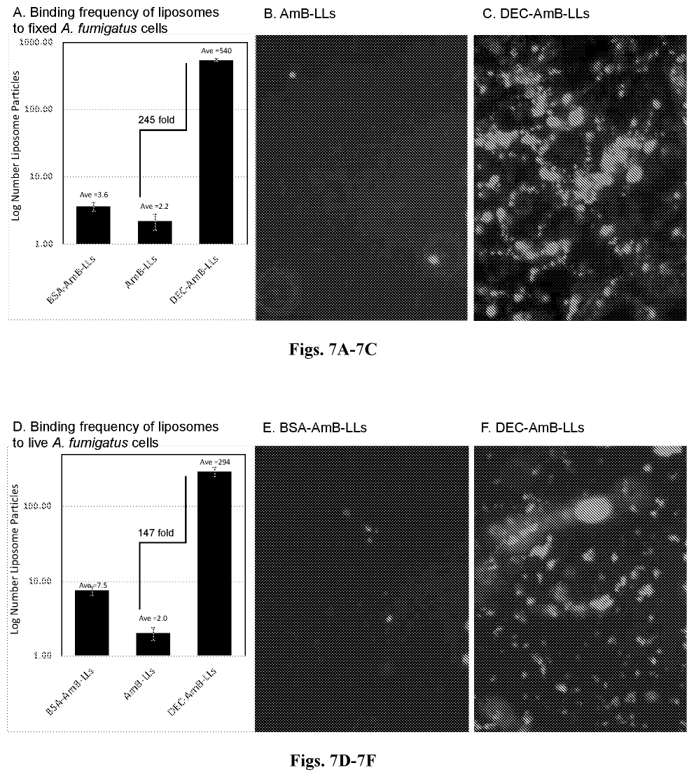

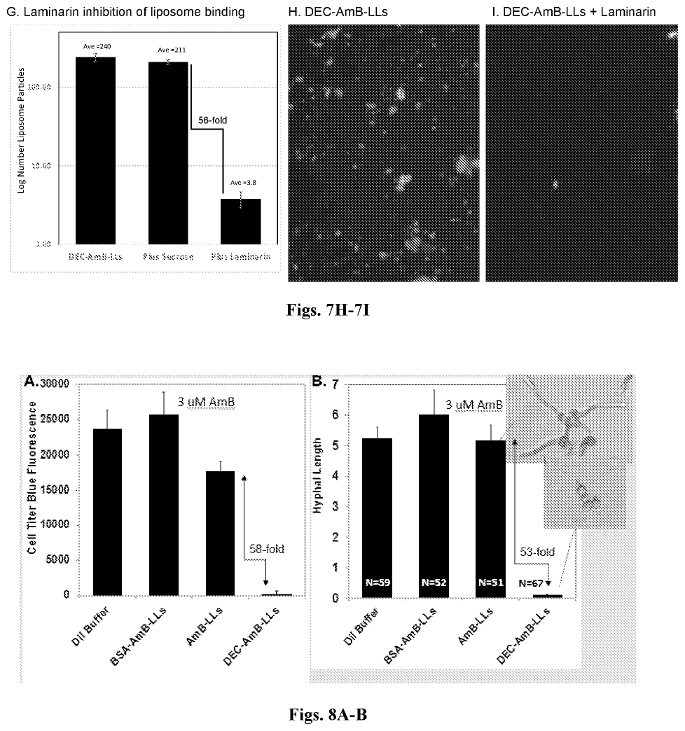

The binding of DEC-AmB-LLs and control LLs to fixed and live fungal cells was quantified by counting the number of individual fluorescent liposomes and clumps of fluorescent liposomes that were bound to dense fields of A. fumigatus hyphae, after washing out unbound liposomes. FIG. 7A-F shows that DEC-AmB-LLs bound to both fixed and live hyphae more than 100-fold more frequently than did control liposomes, BSA-AmB-LLs or AmB-LLs. FIG. 7G-I shows binding of sDectin-1 coated DEC-AmB-LLs is inhibited more than 50-fold by addition of the soluble beta-glucan, laminarin, but not by sucrose, proving that binding to fungal cells is beta-glucan specific.

### Killing and Growth Inhibition of Fungi by DEC-AmB-LLs

Various fungal cell growth and viability assays were performed after treating A. fumigatus with liposomes delivering AmB concentrations near an estimated ED₅₀ of 2 to 3 uM AmB and its estimated MIC of 0.5 to 1 uM for various strains of A. fumigatus. In most of these experiments, 4,500 conidia were germinated and incubated for 36 to 56 hr in 96 well microtiter plates along with drug loaded liposomes. FIG. 8 shows that targeted DEC-AmB-LLs (Dectin-1-coated AmB-Loaded Liposomes) killed or inhibited the growth of A. fumigatus cells far more efficiently than BSA-AmB-LLs or uncoated AmB-LLs. Assays with CellTiter Blue (CTB) reagent showed that treating cells with DEC-AmB-LLs delivering 3 uM AmB killed A. fumigatus more than an order of magnitude more effectively than AmBisome-like AmB-LLs or BSA coated liposomes BSA-AmB-LL, carrying the same amount of drug (FIG. 8A). CTB reagent assays total cytoplasmic esterase activity as a proxy for cell integrity and viability. As a second method to score liposomal activity, hyphal length was examined. Hyphal length assays gave a surprisingly similar result, showing that DEC-AmB-LLs delivering 3 uM AmB were far more effective at inhibiting hyphal growth than AmB-LLs or BSA-AmB-LLs (FIG. 8B). In a complete biological replicate experiment, using a different AmB remote loading method, independent s-Dectin-1 and BSA, and rhodamine loading, and a different liposomal dilution buffer a similar, but slightly less dramatic result was obtained when delivering 3 uM AmB (FIGS. 8C and 8D). CTB reagent and hyphal length assays showed that DEC-AmB-LLs were nearly an order of magnitude more effective at killing or inhibiting the growth of A. fumigatus than AmB-LLs.

An additional assay of liposomal AmB activity was employed, which measured the percent of conidia that germinated in the presence of the various liposomal preparations (FIGS. 8E and 8F). DEC-AmB-LLs delivering as little as 0.09 uM and 0.187 uM AmB inhibited the germination of A. fumigatus conidia a few fold more effectively than AmB-LLs or BSA-AmB-LL.

Using a fourth assay of liposomal activity the endogenous fluorescent green signal produced by the EGFP expressing AEK012 strain of A. fumigatus (FIG. 9) was examined. DEC-AmB-LLs delivering either 2 uM or 0.67 uM AmB were again more effective at inhibiting fungal cell growth than AmB-LLs or BSA-AmB-LLs, when assaying levels of green fluorescence. In summary, various assays demonstrated that targeted DEC-AmB-LLs killed or inhibited the growth or germination of A. fumigatus cells far more efficiently than uncoated AmB-LLs or BSA coated BSA-AmB-LLs.

A dose response curve assaying the percent germination of conidia shown in FIG. 8G, illustrates a wide separation in the performance of the three types of liposomes. DEC-AmB-LLs outperform the other two types of liposomes and activity is proportional to concentration and they out-perform AmB-LLs over a wide range of concentrations. BSA-AmB-LLs always perform the most poorly, perhaps because they block the random access of liposomal membranes to fungal plasma membranes that allow AmB-LLs to bind. The timing of these assays affects the precise shape of these curves, where assays performed early better resolve differences at the lowest concentrations and longer incubations resolve differences in the highest concentrations of AmB by giving more highly inhibited cells to grow. This made it difficult to assay the optimal differences among the liposome preparations over the entire range of concentrations in which DEC-AmB-LLs outperform control liposomes. But overall, the relationships among the three classes of liposomes always remain the same.

### Reduced Animal Cell Binding and Toxicity of DEC-AmB-LLs

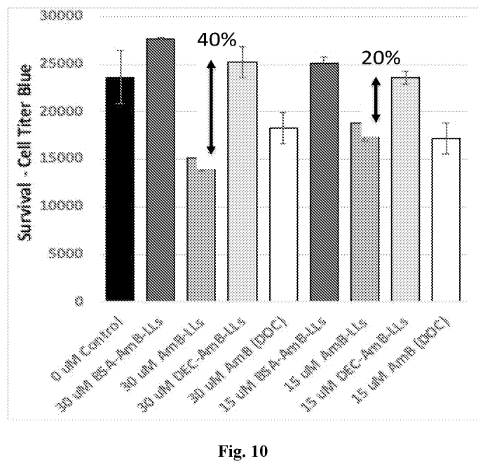

Cell Titer Blue assays for human embryonic kidney HEK293 cell viability showed that AmB-LLs and AmB in a deoxycholate micell suspension were more toxic to HEK293 cells than DEC-AmB-LLs or BSA-AmB-LLs (FIG. 10). Presumably coating liposomes with protein slows their uptake by the plasma membrane of animal cells. Cells were treated for two hours with various preparations delivering 15 or 30 umolar AmB, excess material containing AmB was washed out, grown overnight at 37° C., and then assayed.

### Diagnostics

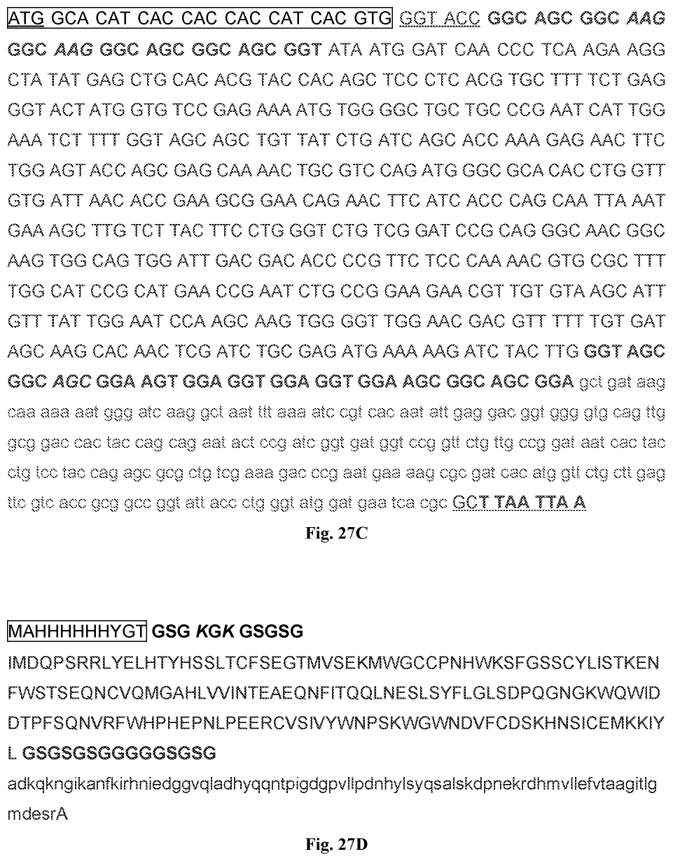

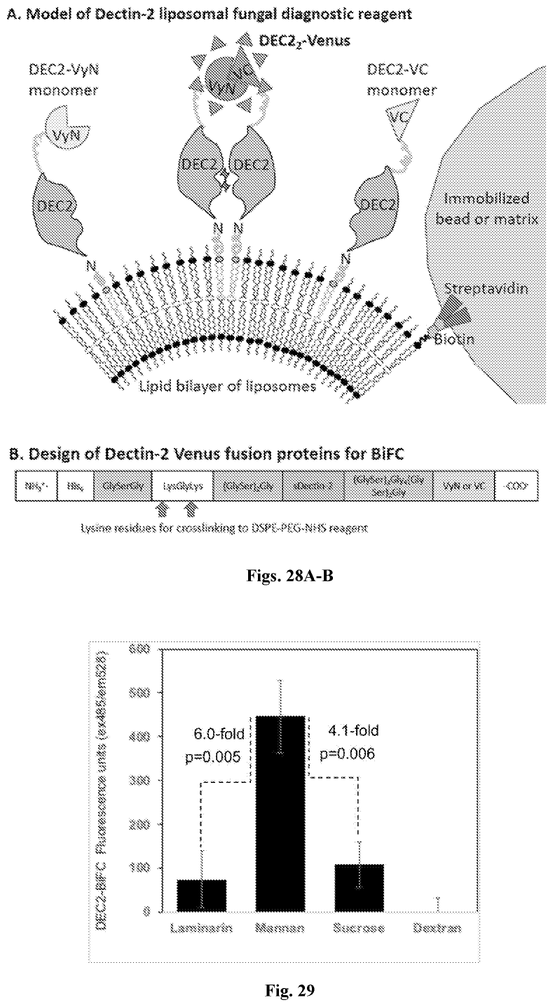

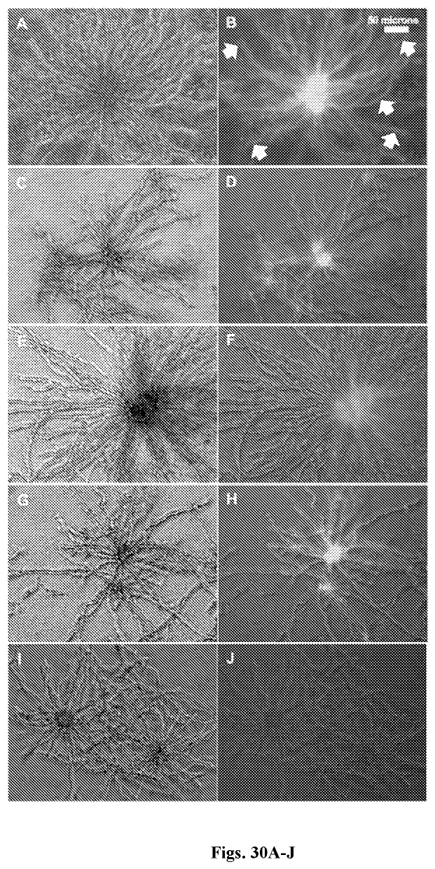

Liposomes coated with 500 targeting molecules, for example, sDectin-1 monomers fused to the N-terminal half of a Venus green fluorescent protein (e.g. DEC1-VN) via a flexible linker and also coated with 500 targeting molecules, for example, sDectin-1 monomers fused via a flexible linker to the C-terminal half of Venus (DEC1-VC), will rapidly recognize and bind low concentrations of fungal beta-glucans to form sDectin-1 dimers and assembled Venus, producing a strong green Bimolecular Fluorescence Complementation (BiFC) signal. See SEQ ID Nos: 13, 14 15, 16 (FIGS. 1M, 1N, 1O, and 1P) for the DNA and protein sequences of these exemplary BiFC reporter constructs. See also, SEQ ID NOs: 17, 18, 19, 20 (FIGS. 1Q, 1R, 1S, 1T, and diagram in FIG. 27D) for Dectin-2 BiFC fusion for additional sequences. In any of the constructs provided herein, an N-terminal or C-terminal fragment of a targeting sequence can be used. For example, an N-terminal or C-terminal fragment of Dectin-1, Dectin-2 or Dectin-3. In some examples, the presence of hundreds or thousands of sDectin-1 monomers on each liposome ensures the rapidity and sensitivity of detection. This assay relies on the fact that Dectins only bind tightly and irreversibly to glucans or mannans on cells or in solution as dimers.

Optionally, when these fungal targeted liposomes are attached to an insoluble matrix, they can detect even lower concentrations of a fungal cell surface molecule, for example, beta-glucans, in large volumes in vitro, in control buffers and in serum, using standard fluorescence instrumentation. Binding to an insoluble matrix keeps the fluorescent signal concentrated in a small volume for more sensitive detection as they are exposed to large volumes of sample material. In addition to serum, immobilized liposomes could assay fungi and soluble fungal cell wall material in urine, lung lavage fluids, or solubilized tissue extracts.

In some examples, targeted liposomes can be used to detect fungal mannans. This can be achieved by, for example, coating liposomes with about 500 sDectin-2 monomers fused to the N-terminal half of a fluorescent protein (e.g., Venus DEC2-VN) and coating the same liposomes with about 500 sDectin-2 or Dectin-3 monomers fused to the C-terminal half of a complementary fluorescent protein (Venus, DEC2-VC, DEC3-VC). Homodimers of Dectin-2 bind strongly to fungal wall mannans, but monomers do not bind significantly. Heterodimers between Dectin-2 and Dectin-3 could bind mannans more strongly than their individual homodimers.

This system will enable assays (1) to be completed within 60 min of combining serum with the liposome matrix, (2) to be completed in one step, (3) to be highly sensitive, detecting very low concentrations of polysaccharide, (4) to be inexpensive, (5) to require minimal operator training and expertise, and/or (6) to detect nearly all fungal pathogens. These liposomes should enable one step assays of invasive fungi in blood, serum, urine, lung exudate, lungs, eyes, throat, vagina, skin, and fingernails and toenails. FIG. 11 depicts one possible model of free floating or immobilized Dectin-coated liposomes for the one-step detection of fungal beta-glucan and mannan polysaccharides. By one-step detection is meant that binding of liposomes to target cell wall components and signal generation are biochemically linked, i.e., detection occurs without the need for additional processing steps or reagents.

The biochemical production of mouse or human sDectin-1 has been complicated, because the proteins easily aggregate and become insoluble and inactive in aqueous buffers. As shown herein, the solubility problems of sDectin-were overcome by combining a variety of approaches including, the use of a very short charged peptide tag, the inclusion of 6 M GuHCl during protein extraction, purification, and chemical modification, by performing renaturation, liposomal loading, and storage in buffers containing a protein solubilizing agent, 1M arginine, and the inclusion of sulfhydryl reducing agents.

Previous reports showed that mouse sDectin-1 binds efficiently to A. fumigatus swollen conidia and germ tubes, but inefficiently if at all to mature hyphae and not at all to un-germinated conidia (Steele et al. The beta-glucan receptor dectin-1 recognizes specific morphologies of Aspergillus fumigatus. PLoS Pathog 1:e42 (2005)). Lack of hyphal binding may be due to greatly reduced beta-glucan levels on the surface of older resting cells or perhaps beta-glucans are less accessible in the mature cell wall. Herein, sDectin-1 coated fluorescent DEC-AmB-LLs bound efficiently to swollen conidia, germ tubes, and a subset of hyphae, suggesting the modified sDectin-1 described herein presented to the surface of liposomes retained its normal affinity for beta-glucans. These binding data showed for the first time that a chemically modified form of sDectin-1 (e.g., DSPE-PEG-Dectin-1) can preserve its fungal cell binding specificity. Furthermore, fluorescent DEC-AmB-LLs binding was rapid and remained stably bound to cells for weeks. Purified sDectin-1 is reported to bind to C. albicans round yeast cells and in hyphae in the region between parent cell and the mature bud, and not to hyphae. Although binding of DEC-AmB-LLs to C. albicans was inefficient as compared to binding of A. fumigatus hyphae, we showed reasonable binding of DEC-AmB-LLs at multiple sites along C. albicans hyphae (FIG. 6). It is possible that the greater avidity of liposome coated with more than a thousand sDectin-1 molecules insured the rapid binding, and very slow release of bound liposomes, parallel to the avidity of pentameric IgM antibody. The presence of thousands of rhodamine molecules on each liposome can also increase the chance of detecting unambiguous fluorescent signals. In a large number of experiments using different binding buffers including BSA blocker and various incubation periods significant affinity of uncoated AmB-LLs or BSA-AmB-LLs to fungal cells was never detected. Although, in preliminary experiments in which BSA blocker was not included in the incubation, BSA-AmB-LLs bound weakly to A. fumigatus swollen conidia.

Aspergillus, Candida and Cryptococcus species belong to three evolutionarily disparate groups of fungi, the Hemiascomycetes, Euascomycetes, and Hymenomycetes, respectively, which are separated from common ancestry by hundreds of millions of years. DEC-AmB-LLs bound specifically to all three suggesting the beta glucans found in the outer cell wall of many pathogenic fungi will be accessible to sDectin-1 targeted liposomes. However, Dectin-1 binds to Candida relatively weakly compared to Aspergillus, whereas Dectin-2 binds more strongly. This suggests that robust pan-fungal detection of fungal pathogens may require assays that detect both glucans and mannans.

As shown herein, in various biological and experimental replicate experiments using distinct cell assay methods, DEC-AmB-LLs killed or inhibited A. fumigatus cells more efficiently than AmBisome-like AmB-LLs delivering the same level of AmB. In all of our experiments, DEC-AmB-LLs were from several fold to more than an order of magnitude more fungicidal than control liposomes over a wide variety of AmB concentrations tested that were near or below the estimated ED₅₀ of 3 uM. Significantly stronger activity of DEC-AmB-LLs over AmB-LLs, even at AmB concentrations as low as 0.094 uM AmB, well below AmB's MIC, was detected. These studies show that DEC-AmB-LLs significantly decrease the ED₅₀ and MIC of AmB for killing and inhibiting the growth of A. fumigatus.

The lipid membrane of uncoated liposomes binds passively to the lipid membrane of animal cells, which unlike fungal cells, are not protected by a cell wall. However, protein coating of liposomes and coating with mouse or human serum albumin, in particular, reduces the clearance of liposomes from animal models, and hence, increases liposome half-life. Presumably protein coating slows the direct interaction of the liposomal membrane with the plasma membrane, to reduce passive delivery of drugs. Protein coating of liposomes with sDectin-1 may reduce the direct interaction of the liposomal membrane with the plasma membrane of animal cells, and hence, reduce their interaction with and fusion to animal cells. The results described herein, showing reduced toxicity of DEC-AmB-LLs relative to AmB-LL, to human kidney cells are consistent with this view.

In summary, sDectin-1, when optionally conjugated to a pegylated lipid carrier and inserted as a monomer into liposomes is able to form functional complexes and efficiently bind beta-glucans in the cell walls of diverse fungal species. Multiple growth inhibition and viability assays on DEC-AmB-LLs delivering AmB concentrations from 0.094 to 3 uM suggest that sDectin-1 targeted liposomes lowered the ED50 of liposomal AmB well below that reported for untargeted AmBisomes as modeled by our AmB-LLs. Furthermore, DEC-AmB-LLs had less affinity for and were less toxic to animal cells than AmBisome-like liposomes. Taking these results altogether, it is reasonable to use sDectin-1 coated liposomes as pan-fungal carriers for targeting antifungal therapeutics.

### Example 2

### Dectin-2

### Cell Culture

C. albicans CAI4 expressing GFP under control of the ADH1 promoter, A. fumigatus A1163, and wild type C. neoformans H99-alpha were grown in Vogel's Minimal Media (VMM)+1% glucose (85)+0.5% BSA or RPMI 1640 media with no red indicator dye (ThermoFisher SKU— 11835-030)+0.5% BSA or YPD (1% yeast extract, 2% peptone, 2% dextrose) in liquid with shaking or on 24 or 96 well polystyrene microtiter plates or on glass microscope chamber slides and incubated at 37° C. for 3 to 36 hr. Plates were pre-coated with poly-L-lysine for A. fumigatus and glass microscope slides were pre-coated with for all three species. All fungal cell growth was carried out in a BSL2 laboratory. Before cells were treated with fluorescent liposomes for microscopic analysis of binding, fungal cells were washed thrice with PBS, fixed in 4% formaldehyde in PBS for 60 mins, washed thrice, and stored at 4° C. in PBS.

The human colorectal adenocarcinoma cell line HT-29 (ATCC HTB-38) and human embryonic kidney cell line HEK-293 (ATCC CRL-1573) were grown in 96 well microtiter plates in RPMI media lacking red indicator dye plus 10% fetal calf serum in a 37° C. incubator in air supplemented with 5% CO₂. Their viability and metabolic activity after overnight antifungal treatment were assayed using CTB reagent diluted 1:10 into the media and incubating for 60 to 90 min at 37° C., 8 wells per treatment.

Production and Chemical Modification of sDectin-2

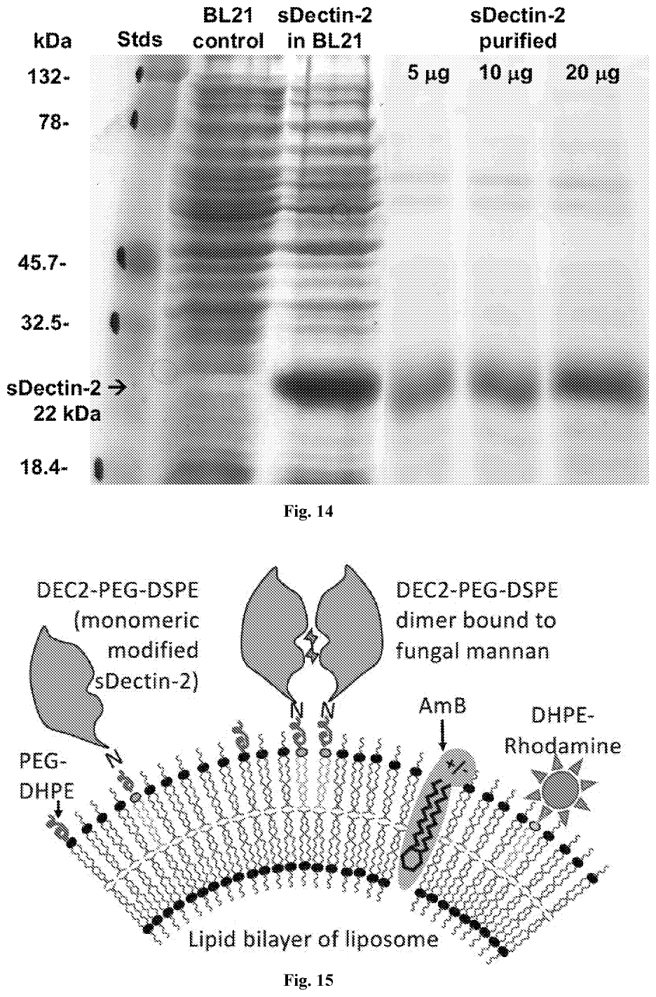

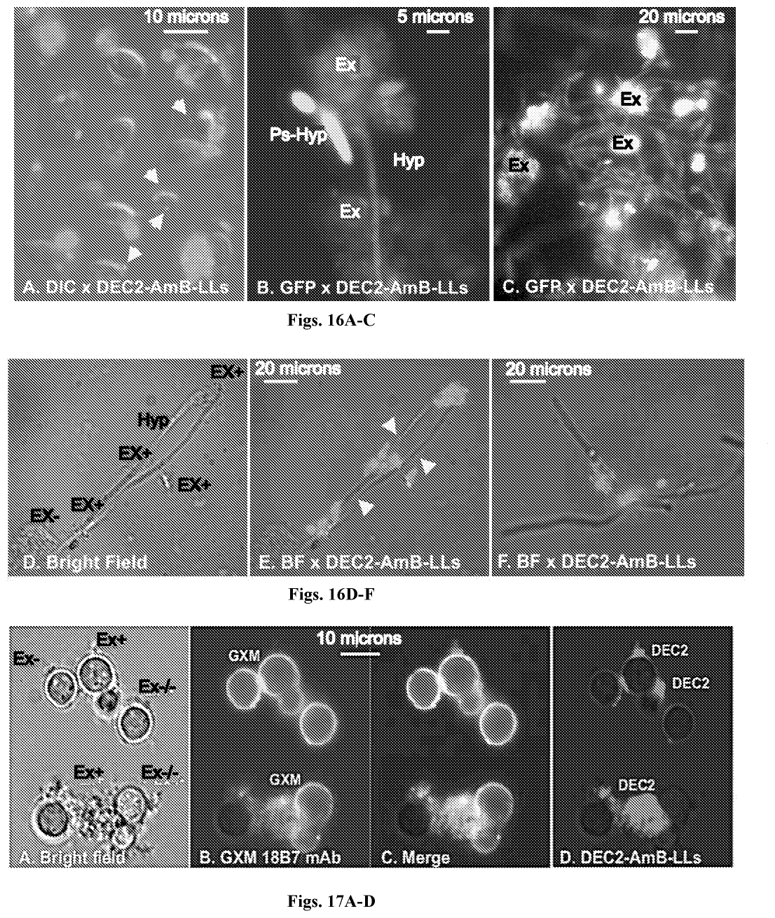

The carboxy terminal end of Dectin-2 contains its mannan-recognition domain, sDectin-2. The sequence of the codon-optimized E. coli expression construct with MmsDectin-2lyshis synthesized and cloned into pET-45B by GenScript is shown in FIG. 13. The 577 base pair long DNA sequence encodes a slightly modified 189 a.a. long sDectin-2 protein containing a vector specified N-terminal (His)₆ affinity tag, an additional flexible GlySer spacer, the sequence LysGlyLys with lysine residues for crosslinking, another flexible spacer followed by the C-terminal 166 a.a. long murine sDectin-2 domain. The modified sDectin-2 protein, DEC2 was expressed in E. coli and purified as described above for mouse sDectin-1 followed by an extra step of gel exclusion chromatography on Sephacryl S-100 HR (GE Healthcare, #17061210). The protein is shown on an SDS PAGE gel stained with Coomassie Blue in FIG. 14. Samples of sDectin-2 at 5 μg/uL in this same GuHCl buffer with freshly added 5 mM 2-mercaptoethanol were adjusted to pH 8.3 with 1 M pH 10 triethanolamine and reacted with the reactive succinimidyl ester NHS moiety of a 4-molar excess of DSPE-PEG-3400-NHS (1,2-Distearoyl-sn-Glycero-3-Phosphoethanolamine (DSPE) conjugated polyethylene glycol (PEG), from Nanosoft polymers, 1544-3400) for 1 hr at 23° C. to make DEC2-PEG-DSPE (Supplemental FIG. 51). Size exclusion chromatography through Bio-Gel P-6 acrylamide resin (Bio-Rad #150-0740) in renaturation and storage buffer RN #5 (0.1 M NaH2PO4, 10 mM Triethanolamine, pH 8.0, 1 M L-Arginine, 100 mM NaCl, 5 mM EDTA, 5 mM 2-mercaptoethanol) removed un-incorporated DSPE-PEG and GuHCl (51). BSA-PEG-DSPE was prepared from BSA (bovine serum albumin, Sigma, A-8022) by the same protocol, but with buffer lacking the GuHCl during DSPE-PEG labeling and L-arginine during Bio-Gel P6 chromatography. Rhodamine labeled sDectin-2 (DEC2-Rhod) was prepared by the same procedure used to make DEC2-PEG-DSPE in the same GuHCl buffer, but labeling was performed with a 4 molar excess of Rhodamine-NHS reagent (Thermo Fisher #46406) over sDectin-2. Hydrolyzed unbound rhodamine reagent and unwanted salts were removed from DEC2-Rhod using size exclusion chromatography on Bio-Gel P2 resin in RN #5 buffer. As with DEC2-PEG-DSPE, RN #5 hold DEC2-Rhod in a state in which is it readily renatured into an active carbohydrate binding form, when diluted into milder biological buffers.

Remote Loading of AmB, sDectin-2, BSA, and Rhodamine into Liposomes

Starting with sterile pegylated liposomes from FormuMax Sci. Inc. (DSPC:CHOL:mPEG2000-DSPE, FormuMax #F10203A) small batches of liposomes with 11 moles percent AmB (AmB, Sigma A4888) relative to 100% liposomal lipid were prepared to make AmB-LLs as described above (see Table 1).

TABLE 1

| | | | DEC2- | BSA- | AmB DOC |
|---|---|---|---|---|---|
| | Gilead's | AmB-LL | AmB-LL | AmB-LL | Micelles |
| Compound | AmBisome ® | This study | This study | This study | This study |
| Additions to liposomes remotely loaded | | | | | |
| for this study presented as moles percent | | | | | |
| of total liposomal lipids | | | | | |
| sDectin-2 (DEC2-PEG-DSPE) | | 0.0 | 1.0 | 0.0 | |
| BSA (BSA-PEG-DSPE) | | | | 0.3 | |
| Amphotericin B | 10.6 | 11.0 | 11.0 | 11.0 | 54.5 |
| Fluconazole | | | | | |
| Rhodamine Lissamine (DHPE-Rhodamine) | | 2.0 | 2.0 | 2.0 | |
| Moles percent in addition to total | 10.6 | 13.0 | 14.0 | 13.3 | 54.5 |
| liposomal lipids | | | | | |
| Lipids, lipidic vitamines, and | | | | | |
| PEG-conjugated lipids defined | | | | | |
| as 100% total liposomal lipids | | | | | |
| mPEG2000-DSPE (N-(Carbonyl- | | 5.0 | 5.0 | 5.0 | |
| methoxypolyethylene glycol 2000)- | | | | | |
| distearoyl-glycerophosphoethanolamine) | | | | | |
| alpha-Tocopherol (form of Vitamine E) | 0.0003 | 0.0 | 0.0 | 0.0 | |
| HSPC (soy phosphotidylcholaine) | 52.7 | 0.0 | 0.0 | 0.0 | |
| DPPC(1,2-Dipalmitoyl-sn-glycerol- | | | | | |
| 3-phosphocholine) | | | | | |
| DPPE ((1,2-Dipalmitoyl-sn-glycerol- | | | | | |
| 3-phosphoethanolamine) | | | | | |
| DSPC (1,2-Distearol-sn-glycerol- | | 50.0 | 50.0 | 50.0 | |
| 3-phosphocholine) | | | | | |
| DSPG (1,2-Distearoyl-sn-glycero- | 21.1 | 0.0 | 0.0 | 0.0 | |
| 3-phosphoglycerol) | | | | | |
| CHOL (Chlosterol) | 26.2 | 45.0 | 45.0 | 45.0 | |
| Sodium deoxycholate (DOC)in micelle | | | | | 100.0 |
| Total liposomal lipids or DOC = 100% | 100 | 100.0 | 100.0 | 100.0 | |

The DEC2-PEG-DSPE and BSA-PEG-DSPE conjugates in RN #5 buffer and PBS, respectively, were integrated via their lipid DSPE moiety into the phospholipid bilayer membrane of AmB-LLs by 30 min incubation at 60° C. to make DEC2-AmB-LLs and BSA-AmB-LLs, parallel to the protocol described above. During these same 60° C. incubations, 2 moles percent of the red fluorescent tag, Lissamine rhodamine B-DHPE (Invitrogen, #L1392) was also incorporated into sDectin-2- and BSA-coated liposomes and AmB-LLs. A duplicate sample of the DEC2-AmB-LLs was subjected to gel exclusion chromatography over Bio-Gel A 0.5 M resin (Bio-Rad, #151-0140). Fluorescent liposomes were efficiently excluded from the resin. Because no sDectin-2 (2.2 OD A₂₈₀/mg/mL) or rhodamine were detected in the low molecular weight fractions included in the gel, we concluded that both were efficiently loaded into liposomes. Fresh 2 mM BME was added to DEC2-AmB-LLs before each use in binding or killing assays. DEC2-AmB-LLs stored at 4° C. in RN #5 appeared to retain full fungal cell binding specificity and killing activity for at least 12 months.

### Microscopy of Liposomes and DEC2-Rhod Bound to Fungal Cells

Formalin fixed fungal cells were incubated with liposomes at 23° C. in liposome dilution buffer LDB2 (20 mM HEPES, 10 mM Triethoanolamine, 150 mM NaCl, 10 mM CaCl₂, 1 mM beta-mercaptoethanol (BME), 5% BSA pH 8.0), wherein the BME was added fresh. Liposomal stocks were diluted 1:200 before incubating with cells such that the sDectin-2 protein concentration was 0.5 μg/100 μL. After 15 min, 1 hr or longer incubations, unbound liposomes washed out with 4 changes of LDB2. Merged images of rhodamine red fluorescent liposomes, green fluorescent cells, and differential interference contrast (DIC) illuminated cells were taken of cells grown on microscope slides under oil immersion at 63× on a Leica DM6000B automated microscope. The DEC2-Rhod stock was also diluted 1:200 before incubating with cells such that the sDectin-2 protein concentration was also 0.5 μg/100 Bright field, DIC, and red (Ex560/Em645) and green (Ex500/Em535) fluorescent images were taken of cells on microtiter plates at 20× on an Olympus IX70 Inverted microscope using an Olympus PEN E-PL7 digital camera and the bight field and/or fluorescent colored layers merged in Photoshop. The area of liposome binding at 20× magnification was quantified by taking an 8-bit grey-scale copy of the unmodified red fluorescent TIF image into Image J (imagej.nih.gov/ij), using Image>Adjust>Threshold>Apply to capture just the red fluorescent areas illuminated by liposomes, and using Analyze>Measure to place the area data for each image in a file. Six to ten photographic images were generally analyzed and averaged for most area estimates. However, because the staining intensity varied widely among photographic fields C. neoformans cells, 90 images were analyzed for each treatment. Bright field, DIC, and fluorescent Images were also taken of cells grown in microsope chamber slides at 20× or under oil immersion at 63× on a Leica DM6000B automated microscope.

The glucuronoxylomannan (GXM) specific monoclonal antibody 18B7 was obtained from Sigma-Aldrich (MABF2069), used at a 1:200 dilution (0.5 ug/100 4), and visualized with secondary goat anti-mouse antibody Alexa488 (Life Technologies, A11001) also diluted 1:200 and photographed with GFP filters (Ex500/Em535).

### Growth Inhibition and Viability Assays Following Liposome Treatment

Liposomal stocks were stored at 615 to 800 μM AmB and typically diluted first 30 to 600-fold into liposome dilution buffer, LDB2 and then diluted 1:11 into growth media to achieve the indicated final fungicide concentrations ranging from 2 μM down to 0.1 μM. Control cells received an equivalent amount of LDB2. CellTiter-Blue (CTB) cell viability and metabolic activity assays of C. albicans and A. fumigatus were conducted as we recently described for A. fumigatus, incubating with CTB reagent for 3 to 4 hr and analyzing 96-well plates in a Bio-Tek Synergy HT fluorescent microtiter plate reader. The fluorescent background from control wells was subtracted from experimental wells. Data from eight wells were averaged for each data point. These assays had a lot of background and were less sensitive if the cells were assayed in VMM. We were unable to detect any fluorescent signal from CTB reagent, when performing parallel cell viability assays on C. neoformans using this protocol or another CTB protocol published for this species. As an alternative measure of C. neoformans and C. albicans cell viability, assays were conducted by growing 1 mL of cells in YPD, adding drug-loaded liposomes for the indicated times of growth, diluting the cells, plating on YPD, and counting colony forming units (CFUs). The fraction of dead C. neoformans cells among cells growing in YPD after treatment with fungicide loaded liposomes was assayed by adding propidium iodide to the media at 50 μg/mL and incubating for 60 min at 37° C. The media was removed and replaced with PBS for fluorescent microscopy using the red fluorescent protein channel (Ex560/Em595) and scoring the percent of dead stained cells relative to the total number of stained and unstained cells. In experiments with DEC1-AmB-LLs, all liposomes were diluted with LDB1 (PBS+5% BSA+1 mM BME) (51) instead of LDB2.

Dectin-2 is encoded by the human and mouse C-type LECtin receptor gene CLEC6A. Dectin-2 binds alpha-mannans and N-linked and O-linked mannans in mannoproteins (42-46). Dectin-2 is expressed in the plasma membrane of some lymphocytes with its mannan binding domain (sDectin-2) on the outside of these cells and it's signaling domain in the cytoplasm. Dectin-2 functions as an innate immune receptor that signals the host of an active fungal infection.

As set forth in Example 1, Dectin-1-coated Amphotericin B (AmB) loaded liposomes were targeted to the beta-glucans in the inner cell walls and bound efficiently to multiple cell types of Aspergillus fumigatus and to C. neoformans yeast cells. Dectin-1-coated AmB loaded liposomes (DEC1-AmB-LLs) efficiently inhibit and kill A. fumigatus cells. However, Dectin-1 liposomes bind poorly to C. albicans, presumably due to the presence of a thick mannan polysaccharide and mannoprotein outer layers masking their beta-glucans. Herein, AmB-loaded liposomes were coated with the mannan-binding domain of mouse Dectin-2, sDectin-2. The sDectin-2-coated AmB-loaded liposomes bound efficiently to C. albicans, C. neoformans and A. fumigatus and dramatically reduced cell growth and viability, relative to untargeted drug-loaded liposomes.

### Results

Preparation of Fungicide-Loaded sDectin-2-Coated Fluorescent Liposomes

A model of the fungicide-loaded, sDectin-2-coated liposomes constructed herein is shown in FIG. 15. Liposome construction methods and liposome composition paralleled closely that of sDectin-1 coated liposomes described above. 11 moles percent of Amphotericin B (AmB) relative to moles of liposomal lipids were remotely loaded into the membrane of pegylated liposomes to make AmB-loaded liposomes AmB-LLs. For reference, the widely used commercial AmB-loaded untargeted liposomal product AmBisome® contains 10.6 moles percent AmB relative to moles of liposomal lipid (Table 1). The murine sDectin-2 sequence was designed to contain a small lysine tag on its amino-terminal end (FIG. 13). It was expressed in E. coli (FIG. 14) and the purified sDectin-2 protein was conjugated via this lysine tag to NHS-PEG-DSPE, making DEC2-PEG-DSPE. DEC2-PEG-DSPE was then incorporated via its DSPE lipid moiety into AmB-LLs, at one mole percent protein molecules relative to moles of liposomal lipid (1,500 molecules sDectin-2 per liposome) to make DEC2-AmB-LLs. Similarly, and as a protein-coated liposomal control, 0.33 moles percent bovine serum albumin was incorporated via a lipid carrier into AmB-LLs to make BSA-AmB-LLs. This resulted in equivalent μg amounts of 22 kDa sDectin-2 and 66 kDa BSA proteins on the surface of these two sets of liposomes. Uncoated AmB-LLs, which closely resemble the commercial product AmBisome®, also served as a liposomal control. Two moles percent DHPE-rhodamine was also incorporated into the liposomal membrane of all three liposome preparations. Hence, all three sets of liposomes contained the same 11 moles percent of AmB and 2 moles percent of rhodamine. The composition of DEC2-AmB-LLs is compared to that of BSA-AmB-LLs, AmB-LLs, AmBisome®, and AmB/Micelles in Table 1.

sDectin-2-Coated Liposomes DEC2-AmB-LLs Bound More Strongly to Diverse Fungal Species than Control Liposomes

sDectin-2-coated, red fluorescent, DEC2-AmB-LLs bound strongly to C. albicans yeast cells, pseudo-hyphae and hyphae (FIG. 16). The vast majority of sDectin-2 coated liposomes bound in large clusters to the extracellular polysaccharide matrix associated with these cells. Furthermore, DEC2-AmB-LLs bound to a large subset of the extracellular matrix (Ex+) surrounding these cells, while some regions of the matrix clearly did not bind sDectin-2 coated liposomes (Ex−) (FIG. 16E). Although 100 nm liposomes are too small to be resolved by light microscopy, the estimated 3,000 rhodamine molecules per liposome (FIG. 15) allows the fluorescent signal from individual liposomes to be visualize. Individual DEC2-AmB-LLs (White arrows, FIG. 15A) or liposome clusters binding directly to C. albicans cell walls were rarely seen. By contrast, individual sDectin-1 coated liposomes bound frequently to the cell wall of A fumigatus cells.

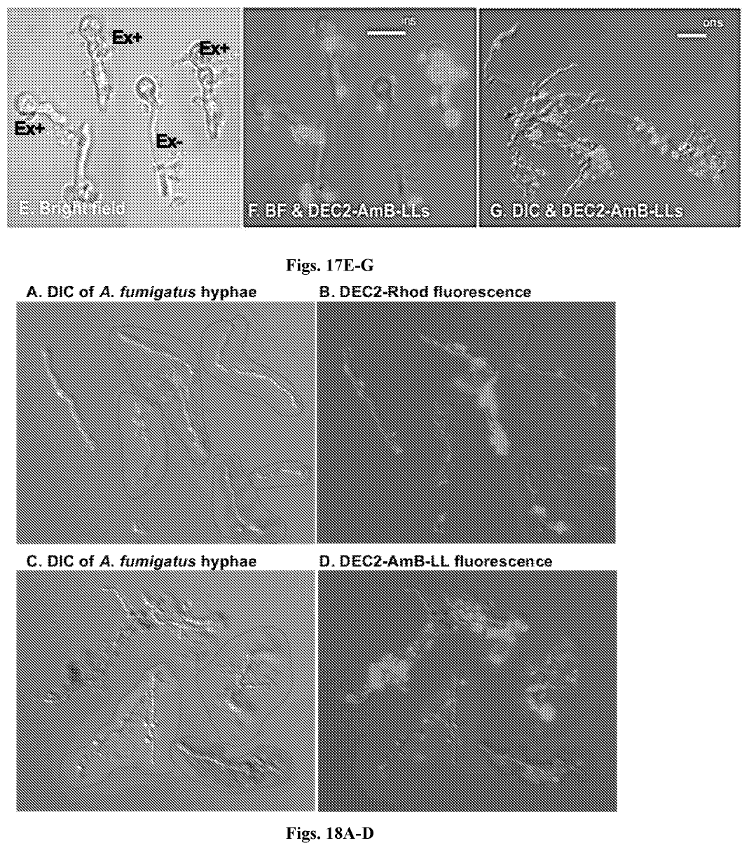

DEC2-AmB-LLs bound strongly to C. neoformans yeast cells (FIG. 17). Monoclonal antibody 18B7 is specific for the glucuronoxylomannan (GXM) found in the capsule and exopolysaccharide of C. neoformans. Antibody 18B7 stained most (FIG. 17B) but not all of the cell capsules and most but not all of the exopolysaccharide that were visible in bright field images (Ex+, FIG. 17A). DEC2-AmB-LLs co-stained strongly to most of the 18B7 stained GXM regions in the exopolysaccharide matrix, but did not stain the 187B-labeled GXM of the capsule (FIG. 17C, 17D). Furthermore, there were some regions of extracellular matrix that did not stain with either 18B7 or DEC2-AmB-LLs (Ex−/−, FIG. 17A).

DEC2-AmB-LLs also bound in large clusters to the exopolysaccharide matrices produced by A. fumigatus germinated conidia and hyphae (FIG. 17E, 17F, 17G). Again, little if any binding was associated with the cell wall itself. Also, there appear to be areas where the expolysaccharide matrix is not stained or poorly stained (Ex−, FIGS. 17E & 17F).

Because the DEC2-AmB-LLs bound poorly or not at all to mannans within the tightly crosslinked polysaccharide of cells walls of all three fungal species examined, we considered that the 100 nanometer diameter size of our liposomes restricted their access or that sDectin-2 was somehow restricted from full activity when presented in a liposomal membrane. The rotational diameter DEC2-AmB-LLs in solution would be even larger then their physical size estimate, due to their coating with sDectin-2 protein and associated water molecules. sDectin-2 itself has an atomic weight of 22 KDa, and hence, a rotational diameter in solution that may be estimated at ˜4 nanometers. Rhodamine coupled sDectin-2, DEC2-Rhod, was prepared. The atomic weight of rhodamine (0.48 kD) and one or two rhodamine coupled molecules will have little effect on this size estimate for DEC2-Rhod. Red fluorescent DEC2-Rhod bound strongly to most of the exopolysaccharide matrix surrounding A. fumigatus hyphal cells (FIG. 18, plates A & B). The pattern of binding and intensity of binding was indistinguishable from that of DEC2-AmB-LLs (FIG. 18, plates C and D).

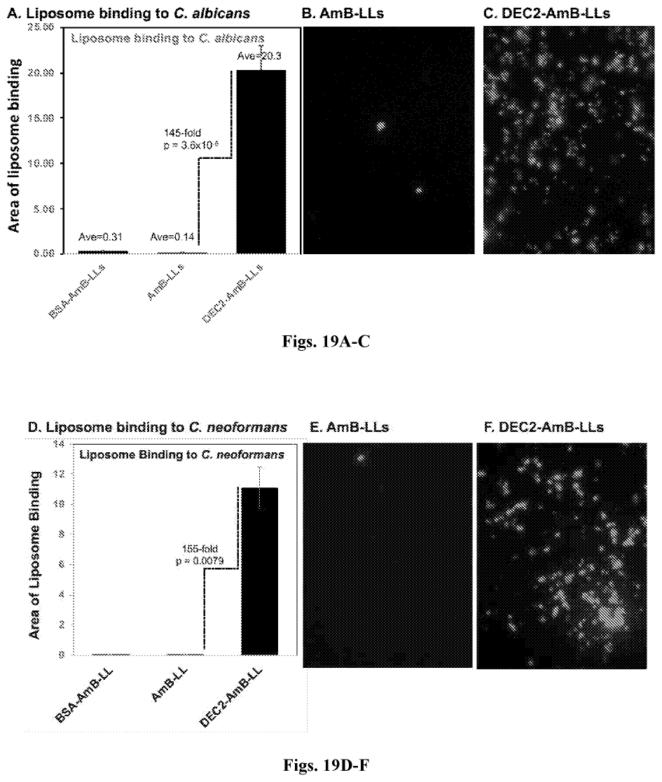

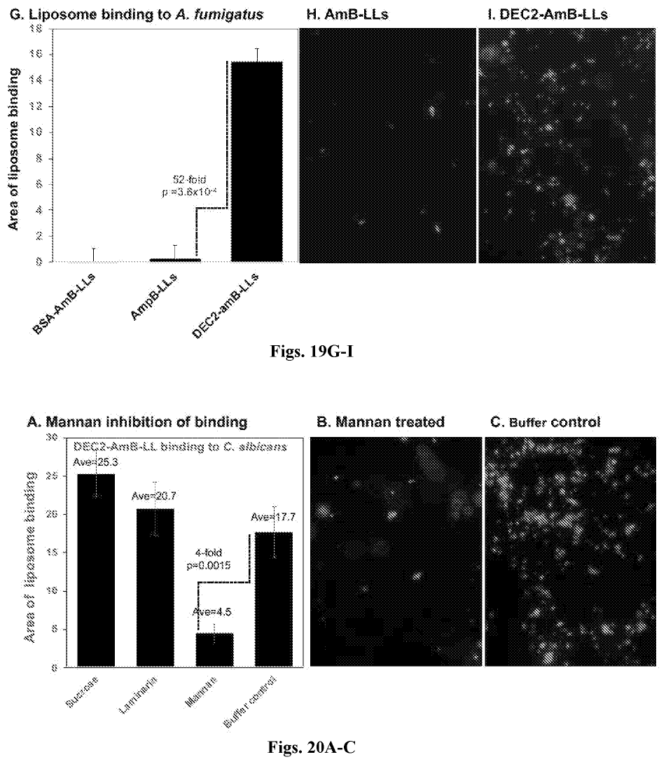

The efficiency of DEC2-AmB-LLs binding to fungal cells as compared to control uncoated AmB-LLs and BSA-coated BSA-AmB-LLs was quantified. The area of fluorescent liposome signal was measured from multiple red fluorescent photographic images taken of liposome stained cultures that were nearly confluent with fungal cells (FIG. 19). DEC2-AmB-LLs bound to C. albicans pseudo-hyphae and hyphae, C. neoformans yeast cells, and A. fumigatus hyphae 50-fold to 150-fold more efficiently than AmB-LLs or BSA-AmB-LLs (FIG. 19A, 19D, 19G). Examples of the photographic images of fluorescent liposomes quantified to make these measurements are presented adjacent to each bar graph (FIG. 19B, 19C, 19E, 19F, 19H, 19I).

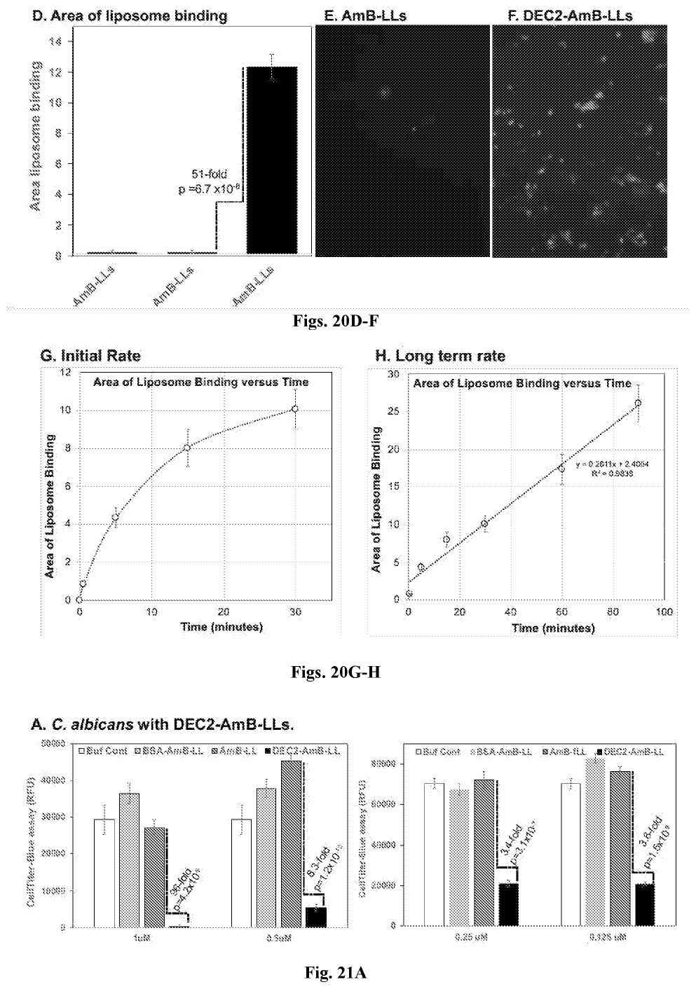

Using this same quantitative assay of the area of fluorescent liposome binding to C. albicans pseudo-hyphae and hyphae the specificity, stability, and rate of DEC2-AmB-LL binding (FIG. 20) was characterized. DEC2-AmB-LLs labeling was 75% inhibited by the inclusion of solubilized yeast mannans during the binding assay, but not by the same concentrations of the soluble beta-glucan laminarin or of the glucose-fructose containing disaccharide sucrose (FIG. 20A, 20B, 20C). This result confirmed that sDectin-2 targeted liposome binding to the extracellular matrix was mannan specific, in agreement with the published carbohydrate specificity of sDectin-2. The stability of DEC2-AmB-LLs binding to C. albicans cells was examined by taking DEC2-AmB-LL stained preparations examined in FIG. 19A, and storing them in the dark in phosphate buffered saline at 4° C. After 2 months, these cells were re-photographed and liposomal staining quantified. The fluorescent intensity of DEC2-AmB-LLs bound to cells remained strong and estimated to be 50-fold stronger than the non-specific binding of AmB-LLs (FIG. 20D, 20E, 20F). This result suggests that the DEC2-AmB-LLs themselves are relatively stable and that their binding to cells is also relatively stable. The rate of DEC2-AmB-LL binding was estimated by exposing dense fields of C. albicans pseudohyphal and hyphal cells to liposomes for periods ranging from 10 seconds to 90 min before washing off unbound liposomes (FIG. 20G, 20H). The area of DEC2-AmB-LLs labeling increased rapidly and exponentially for the first 15 min (FIG. 20G) and then slowed, but did not appear to be complete after 90 min (FIG. 20H).

In summary, Dectin-2-coated AmB loaded liposomes DEC2-AmB-LLs bound specifically, stably, and rapidly to fungal mannans present in the extracellular matrix of C. albicans grown in vitro, while little non-specific binding of control liposomes was observed. There were only trace amounts of DEC2-AmB-LL binding to the cell wall. DEC2-AmB-LLs also bound efficiently to portions of the extracellular matrices surrounding C. neoformans and A. fumigatus cells.

### Growth Inhibition and Killing by DEC2-AmB-LLs

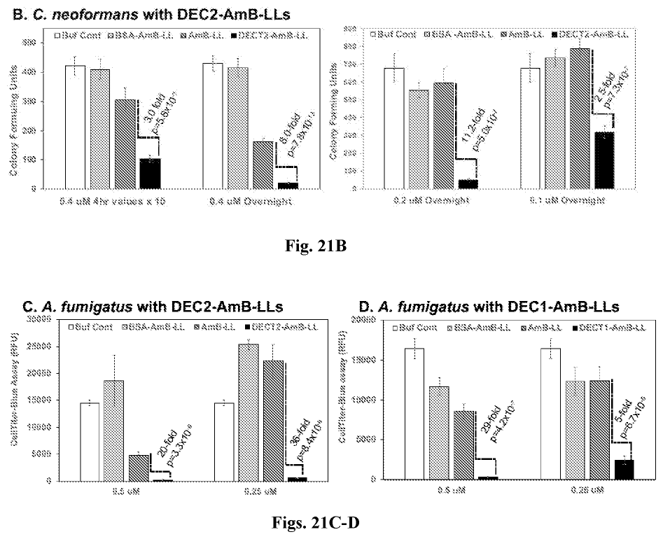

Various fungal cell growth and viability assays were performed after treating actively growing cultures of C. albicans, C. neoformans and A. fumigatus with sDectin-2-coated and control liposomes delivering AmB concentrations near the Minimum Inhibitory Concentrations (MICs) for this fungicide (FIG. 21). Depending upon the assay conditions and delivery method for AmB the MICs for AmB estimated for these species range from 0.06 to 1.3 μM.

4,000 C. albicans yeast cells were inoculated into individual wells of 96 well microtiter plates, grew them for 6 hours to the pseudohyphal and early hyphal stage, and treated cells with drug-loaded liposomes. After a 30 min incubation, liposomes were washed out and cells were grown for an additional 16 hours. FIG. 21A shows that targeted DEC2-AmB-LLs delivering from 1 μM AmB down to 0.125 μM AmB killed or inhibited of C. albicans cells from 90-fold to 3-fold more efficiently than uncoated AmB-LLs or BSA-AmB-LLs delivering the same concentrations of AmB. The difference is remarkable given that in these experiments, cells were exposed to liposomal drug for only 30 min. These data were obtained using CellTiter-Blue reagent to assess cytoplasmic reductase activity as a proxy for cell integrity and viability. Dead cells or metabolically inactive cells do not reduce the resazurin substrate to fluorescent resorufin product. Treating C. albicans cells continuously with liposomes for the entire 16 hr time period or with higher drug concentrations resulted in too much cell death for all three drug loaded liposome samples to clearly resolve differences among the different liposome preparations. Six month old preparations of DEC2-AmB-LLs retained their full antifungal activity, so long as they were freshly reduced. Viable cell numbers after liposome treatment were also assayed. C. albicans yeast cells growing in rich media in liquid culture were incubated with liposomes delivering 2 μM AmB. The liposomes were washed out after 30 min. After an additional 6 hr of growth, the cultures were diluted and assayed for colony forming units (CFUs) on agar plates with rich media. Based on CFUs, DEC2-AmB-LLs were 3-fold more effective at inhibiting or killing C. albicans yeast cells in liquid than AmB-LLs or BSA-AmB-LLs (FIG. 13A).

C. neoformans yeast cells growing in liquid were treated for 4 hours with liposomes delivering 0.4 μM AmB and overnight with liposomes delivering 0.4, 0.2 and 0.1 μM AmB (FIG. 21B). At the end of each treatment, cells were diluted and colony forming units (CFUs) were assayed on agar plates with rich media. DEC2-AmB-LLs were 2.5-fold to 11-fold more effective at killing C. neoformans than AmB-LLs or BSA-AmB-LLs, with the optimum treatment being 0.2 μM AmB overnight. As an alternative assay, C. neoformans yeast cells growing on microtiter plates were treated for 5 hours with liposomes delivering 1 μM AmB. Cells were immediately assayed for cell death by incubating them with propidium iodide. Propidium iodide enters dead, but not live cells, and fluoresces red when it intercalates into double stranded DNA or short regions of double stranded RNA. Propidium iodide assays showed that under these treatment conditions DEC2-AmB-LLs were 5-fold more efficient at killing C. neoformans cells than uncoated AmB-LLs (FIG. 13B, 13C, 13D).

A. fumigatus was treated with liposomes delivering AmB concentrations near and below MICs estimated for AmBisome®, 0.5 Conidia were germinated and grown until the very early germling stage, when the germ tube first began to emerge from 95% of conidia. Cells were then treated for 2 hr with AmB-containing liposomes or liposomal dilution buffer and unbound liposomes washed off with growth media. Cells were grown an additional 19 hr and assayed for viability and metabolic activity with CellTiter Blue reagent. DEC2-AmB-LLs delivering 0.5 μM and 0.25 μM AmB killed or inhibited the growth of A. fumigatus 20-fold and 36-fold more efficiently than AmB-LLs, respectively (FIG. 21C). It should be noted that the dilution buffer control treated A. fumigatus cells overgrow and generate a thick hyphal mat in the microtiter wells during this assay. Hence, the metabolic activity and CellTiter Blue signal from these control cells was low.

Dectin-1 targeted AmB-loaded liposome also bind efficiently to A. fumigatus swollen conidia, germlings, and hyphal cells and inhibit and kill these cells, although they are binding beta-glucans and not alpha-mannans. In this previous study, cells were incubated continuously with liposomes during the entire assay and not washed out. For a more direct comparison of the drug targeting efficiency of the two Dectins, DEC1-AmB-LLs were examined using the same assay design used herein for DEC2-AmB-LLs, washing out the liposomes after 2 hrs, except that liposomes were initially diluted into LDB1 and the cells were grown out for 16 hr. DEC1-AmB-LLs delivering 0.5 μM and 0.25 μM AmB killed or inhibited the growth of A. fumigatus 28-fold and 5-fold more efficiently than AmB-LLs, respectively (FIG. 21D). Using this assay condition, the results from AmB-loaded liposomes targeted by Dectin-1 and Dectin-2 are very similar.

Toxicity of DEC2-AmB-LLs to animal cells Rapidly growing cultures of human HEK 293 and human HT-29 cells were treated overnight with various liposomes and a deoxycholate micelle suspension each delivering 15 μM AmB. Based on CellTiter-Blue assay of metabolic activity and viability, DEC2-AmB-LLs were 10% to 20% more toxic than AmB-LLs or BSA-AmB-LLs and 2- to 5-fold less toxic than AmB deoxycholate (AmB/DOC) micelles (FIG. 14). When these cells were treated to receive lower AmB concentrations, for example, 3 μM AmB, only AmB/DOC micelles showed measurable toxicity. None of the three liposomal preparations appeared to have any particular affinity for either of these cell lines, when examined by fluorescence microscopy.

In summary, the N-terminal domain of Dectin-2, sDectin-2, was coupled to a lipid carrier and this conjugate was inserted into liposomes such that the C-terminal carbohydrate recognition domain (CRD) of each monomer faced outward from the liposomal membrane. Considering the efficient binding observed for these DEC2-AmB-LLs to the extracellular matrices of three diverse human fungal pathogens, the sDectin-2 monomers must have been conformationally free to form the functional dimers necessary for efficient mannan binding, C-type lectin receptors often bind some of their substrates weakly. The published estimated Effective Concentrations for 50% of sDectin-2 binding to mannan-related polysaccharides (EC50s) range from approximately 20 mM for mannose, 2 mM for mannan-a-1-2-mannan, down to 150 μM for mannoglycan. This is indeed weak binding as compared to Dectin-2's closest paralog, Dectin-1, which has EC50s for binding various beta-glucans ranging from 2 mM down to 2.2 picomolar. It is likely that the greater avidity created by having approximately 1,500 sDectin-2 monomers on each liposome resulted in the rapid, strong, and stable binding observed for DEC2-AmB-LL binding to fungal cells.

This data on the efficient binding of DEC2-AmB-LL to the extracellular matrix of all three species is consistent with the mannan rich content of their exopolysaccharides. However, some regions of the C. albicans, C. neoformans and A. fumigatus matrices did not stain or stained poorly with sDectin-2 coated liposomes, suggesting that either the distribution of mannans in the matrix is heterogeneous or the mannans in these regions are masked from exposure to liposomal sDectin-2. It was shown that the binding of DEC2-AmB-LLs to exopolysaccharide mannans was no more restricted than the binding of the much smaller sized DEC2-Rhod, suggesting size restriction may not be the primary limitation.

There was no convincing evidence that DEC2-AmB-LLs bound more than at trace levels to the cell walls of any of these fungal species. Although the cell wall mannan content varies widely based on growth media and methods of chemical analysis, the estimated cell wall mannan polysaccharide content of C. neoformans is 22%, of C. albicans is 40% and of A. fumigatus is 15 to 41%. Although Rhodamine labeled sDectin-2 DEC2-Rhod bound with similar intensity and pattern of to the exopolysaccharide matrix of A. fumigatus as DEC2-AmB-LLs, DEC2-Rhod did not stain the cell wall. Hence, again a size barrier for the much larger DEC2-AmB-LLs does not appear to explain their lack of cell wall binding. Hence, these findings suggest the cell wall mannans may be chemically masked from sDectin-2-mediated liposomal binding. This result parallels the masking of C. albicans cell wall beta-glucans from sDectin-1 binding and from DEC1-AmB-LL binding.

C. albicans reversibly switches among unicellular ovoid yeast and multicellular ellipsoid pseudo-hyphal and elongated hyphal morphologies. All three stages are capable of producing an extracellular matrix and adhering to host tissues. The matrices of all three bound DEC2-AmB-LLs, suggesting fungicide loaded Dectin-2 coated liposomal therapeutics have the potential to reduce the virulence of C. albicans.

DEC2-AmB-LL inhibited and killed C. albicans, C. neoformans and A. fumigatus far more efficiently than plain uncoated AmB-LLs or BSA coated BSA-AmB-LLs delivering the same concentrations of AmB. A combination of metabolic activity assays based on CTB reagent, cell growth assays based on CFUs, and propidium iodide staining of dead cells confirmed that both inhibition and killing of cells occurred. Incubation with DEC2-AmB-LLs for as little as 30 min to a few hours resulted in significant killing. DEC2-AmB-LLs were 3-fold to 90-fold more efficient at inhibiting or killing fungal cells, when delivering AmB concentrations near or below the MIC values reported for AmB, concentrations at which an AmBisome® equivalent uncoated AmB-LLs, had little or no impact on cell inhibition or survival. The DEC2-AmB-LL bound all three species 50- to 150-fold better than AmB-LLs and under some conditions tested inhibited and killed them 11- to 94-fold more efficiently than AmB-LLs.

A significant reduction in the MIC for AmB or other liposomal packaged therapeutic fungicides should result in reducing fungicide doses and the frequency of drug administration, which, in turn, should reduce host toxicity. Shown herein is that DEC2-AmB-LLs were not particularly toxic to animal cells, when delivering 15 μM AmB, which was 15- to 150-fold higher AmB concentrations than used here to kill fungal cells.

These studies were performed with the mouse sDectin-2 protein sequence to avoid problems of sDectin-2 immunogenicity during the future testing of sDectin-2 targeted antifungals in mouse models of candidiasis, aspergillosis, and cryptococcosis. The human sDectin-2 protein sequence is 72% identical to the mouse protein and is only two amino acids shorter. Therefore, manipulating the human protein to target fungicide loaded therapeutic liposomes for use in clinical studies is possible.

In summary, there is a dire need for new antifungal therapeutics. DEC2-AmB-LLs bound efficiently to the extracellular matrices produced by diverse cellular stages of C. albicans, C. neoformans, and A. fumigatus. DEC2-AmB-LLs delivering AmB concentrations near or below AmB's MICs for growth inhibition and killing of all three species showed sDectin-2 targeting of the liposome packaged drug improved the fungicidal effect by an order of magnitude or more over untargeted liposomal AmB. It is reasonable to propose that, drug-loaded liposomes targeted to fungal cells have significant potential as pan-antifungal therapeutics with a wide range of applications.

### Example 3

### Dectin-2 Coated Antifungal-Liposomes Suppress Fungal Burden in a Murine Model of Pulmonary Aspergillosis

### Fungal Cell Culture

The human clinical isolate of A. fumigatus CEA10 (CBS 144.89, ATCC MYA1163) has been used previously in mouse models of aspergillosis (See Desobeaux and Cray, “Rodent Models of Invasive Aspergillosis due to Aspergillus fumigatus: Still a Long Path toward Standardization,” Front Microbiol. 8, 841 (2017)). Conidia were prepared by growing CEA10 for 6 days at 37° C. on 1.5% agar plates containing Vogel's Minimal Media (VMM)+1% glucose+100 ug/mL each of Kanamycin and ampicillin. Conidia were harvested by gently shaking the plate with glass beads and phosphate buffered saline plus 0.05% Tween 20. Conidia were filtered through a sterile 40 micron nylon mesh filter (Fisher Sci. #22363547, Hampton N.H.), settled at 1×g overnight to concentrate conidia, and conidial cell density was determined in a hemocytometer. Germination rates were shown to be close to 100%.

### Antimetabolite and Steroid Preparation

A cyclophosphamide (Cayman #13849) stock of 35 mg/mL was prepared in saline pH 7.4 and delivered at 175 mg/kg mouse body weight. A suspension of cortisone acetate (Cayman Chemical Co., #23798, Ann Arbor, Mich.) was prepared at 22.4 mg/mL in di-water and delivered at 112 mg/kg. A stock of triamcinolone (Millipore Sigma #T6376, Burlington, Mass.) at 40 mg/mL was prepared in DMSO and stored at 4° C. This stock was diluted 1:4 v/v in PBS to prepare an aqueous suspension just prior to intraperitoneal injection of 40 mg/kg.

### Immunosuppression-Mediated Mouse Models of Pulmonary Aspergillosis

Seven-week-old outbred female CD1 (CD-1 IGS) Swiss mice were obtained from Charles River Labs (25 g to 30 g ea.). CD1 mice have been used in the majority of studies of experimental aspergillosis⁵⁶. Steroid model (FIG. 22A): Mice were immuno-suppressed (FIG. 22) by intraperitoneal injection of triamcinolone⁶³ of 40 mg/kg mouse weight on Day −1 (D-1) and on D3. Leukopenic model (FIG. 22B): Mice were immunosuppressed using a single IP injection of 175 mg/kg cyclophosphamide on D-3 and then a single subcutaneous injection of 40 mg/kg triamcinolone on D-1. Because these mice were to be euthanized on Day 4 to assay fungal burden, they were not given subsequent injections of immunosuppressants.

Immunosuppressed mice were infected by oropharyngeal aspiration of 50 μL conidial samples on D1 (FIG. 22). The progression of symptoms began first with a lack of grooming and a ruffled coat and then mild lethargy. Once mice showed severe lethargy and/or they had lost 25% of their body weight, they were declared clinically dead and euthanized by cervical dislocation following anesthesia. For fungal burden experiments all animals were euthanized on D4. All mouse protocols met guidelines for the ethical treatment of non-human animals outlined by the U.S. Federal government and University of Georgia's Institutional Animal Care and Use Committee (IACUC).

Fungal chitin in hand prepared section of mouse lung was stained with calcofluor white RC (Blankophor BBH SV-2560 Bayer, Corp.). A 25 mM stock was prepared by dissolving 5 mg in 218 μL of DMSO and storing in the dark at 4° C. Tissue was stained in a solution prepared by diluting the stock 1:1,000 into PBS+5% DMSO to a final concentration of 25 uM calcofluor white.

Fungal Burden Estimated by Colony Forming Units (CFUs) and Real Time Quantitative PCR (qPCR).

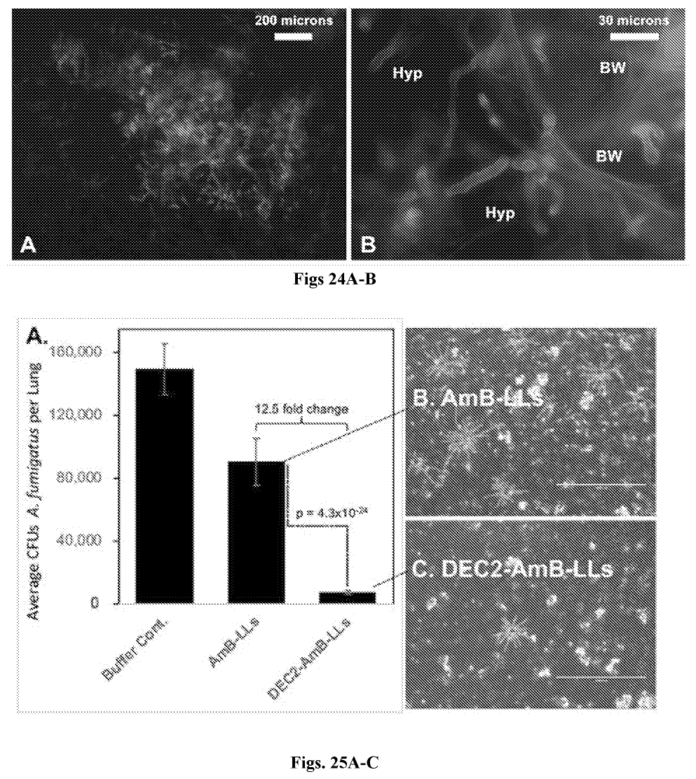

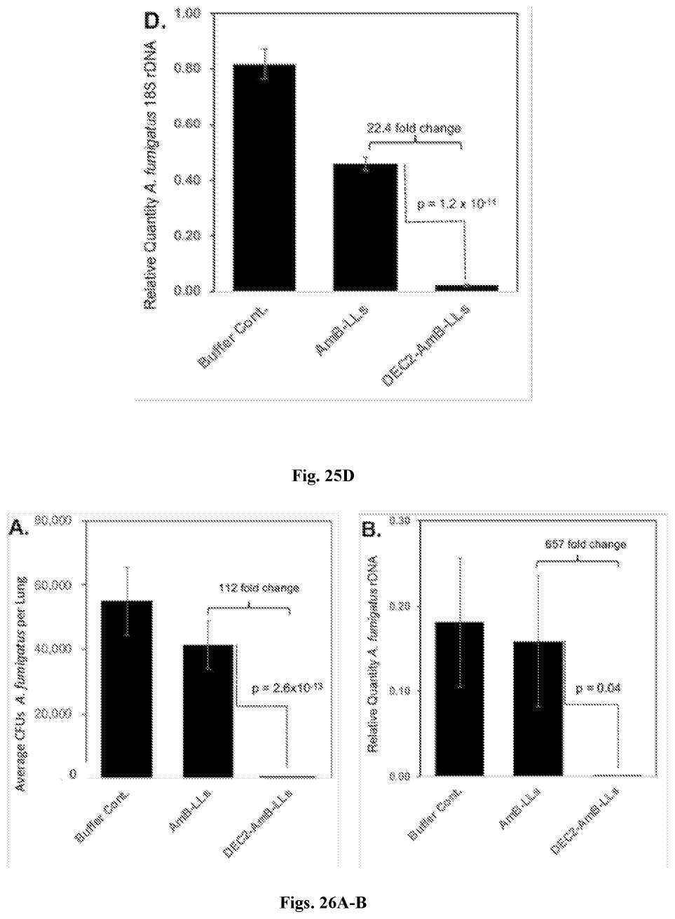

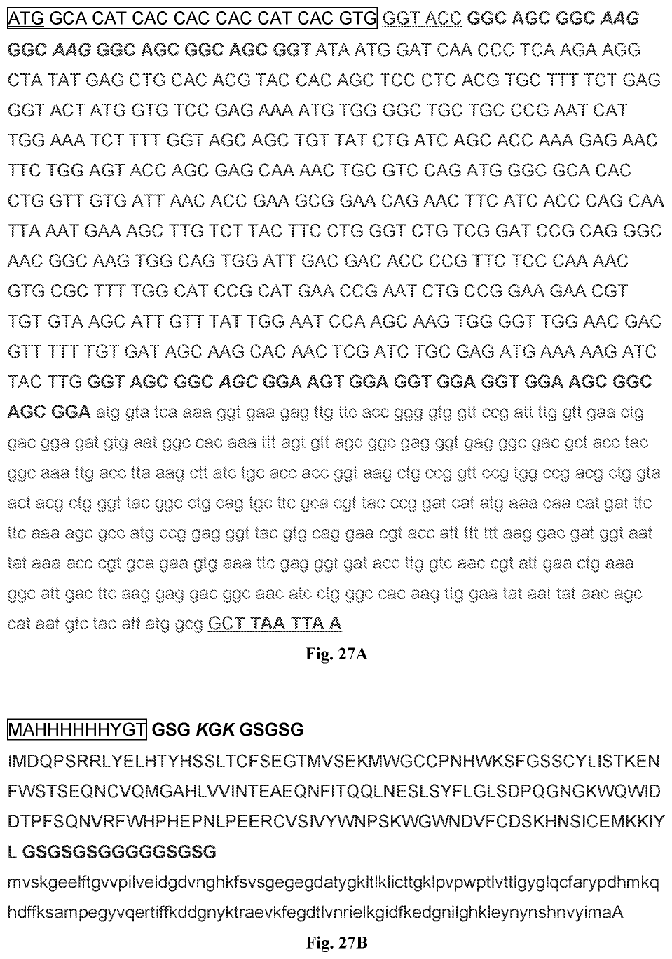

Excised lungs from animals surviving to D4 were weighed and minced into hundreds of approximately 1 mm³ diameter pieces and the pieces were mixed, such that the entire lung could be accurately sampled using a subset of the minced issue. CFUs: 25 mg of lung tissue was homogenized in 200 μL of PBS and spread by shaking with sterile glass beads on YPD (yeast potato dextrose) agar plates containing 100 μg/mL each of Kanamycin and Ampicillin. After a 16 hr incubation at 37° C., the micro fungal colonies were counted and some photographed on an EVOS imaging system at 4× magnification (FIG. 25B, 25C). The number of CFUs reported in FIG. 25A and FIG. 26A was corrected for the area of the entire plate relative to each microscopic field and the weight of each lung. qPCR: DNA was extracted from 25 mg parallel samples from each lung using Qiagen's DNeay® Blood & Tissue Kit (#69504, Hilden, Germany). As per the manufacturer's instructions, the tissue was mixed with 180 uL of buffer ATL and 20 uL proteinase K. At this point the protocol was modified to break fungal cell walls by adding glass beads and shaking the sample a bead beater (RETSCH MM300 Laboratory Mill) at moderate speed for 10 min at room temperature. The homogenate was a transparent liquid suggesting both lung and fungal cells were completely solubilized in the ATL buffer, but it contained some floating lipid. This material was filtered through a Qiagen Shredder Spin Column (Cat. #79654) to remove floating lipid. At this point we returned to the manufacturers protocol for DNA preparation beginning with the recommended 56° C. incubation for 10 min. 10 ug of DNA was typically obtained from 25 mg of lung tissue. Quantitative real-time PCR (qPCR) was used to estimate the amount of A. fumigatus rRNA repeat sequences in a 100 ng sample of lung DNA similar to previously described approaches⁶⁵. Several new primer pairs were designed for the intergenic spacer (IGS) in the rDNA genes of A. fumigatus. The optimal primer pair giving the lowest cycle threshold value (Ct) and a single dissociation peak had the following sequences (Af18SrRNA2S forward primer, 5′-GGATCGGGCGGTGTTTCTATGA and Af18SrRNA2A reverse primer 5′-TTCTTTAAGTTTCAGCCTTGCGACCAT). This primer pair gave no detectable product even after 45 cycles of PCR, when uninfected lung tissue was examined. The Relative Quantity (RQ) of fungal rDNA IGS was determined by normalizing all Ct values to the lowest Ct value determined for a Control infected lung sample using the dCt method (See Livak et al. “Analysis of relative gene expression data using real-time quantitative PCR and the 2(−Delta Delta C(T)) Method,” Methods 25, 402-408 (2001)).

### Results

As described herein, a new delivery system for antifungal drugs using the C-type lectin receptors Dectin-1 and Dectin-2 was developed to target drug loaded-liposomes to the glucans and mannans, respectively, polysaccharide found in the cell wall and exopolysaccharide matrices of most fungal pathogens. Compared to an untargeted AmBisome®-like liposomal drug, in some instances, the Dectin-2 coated-AmB liposomes reduced the in vitro effective dose of liposomal AmB for 90% inhibition and killing of A. fumigatus about 10- to 90-fold. Herein, both steroid and leukopenic models of immunosuppression-mediated mouse pulmonary aspergillosis were employed to study the in vivo efficacy of this new class of therapeutic agent. These experiments were performed near the minimum inhibitory concentration for AmB delivered by AmBisome®, 0.2 mg AmB/kg mouse weight reported in the literature. For both models, herein, it was shown that Dectin-2 targeted AmB-loaded liposomes reduced the fungal burden in lungs by more than one to two orders-of-magnitude relative to untargeted AmBisome®-like liposomes (AmB-LLs) delivering the same low AmB concentrations. By dramatically reducing the effective dose of antifungal drug, targeted pan-antifungal liposomes have the power to create a new clinical paradigm to more safely treat diverse fungal diseases, whether they are invasive or localized infections.

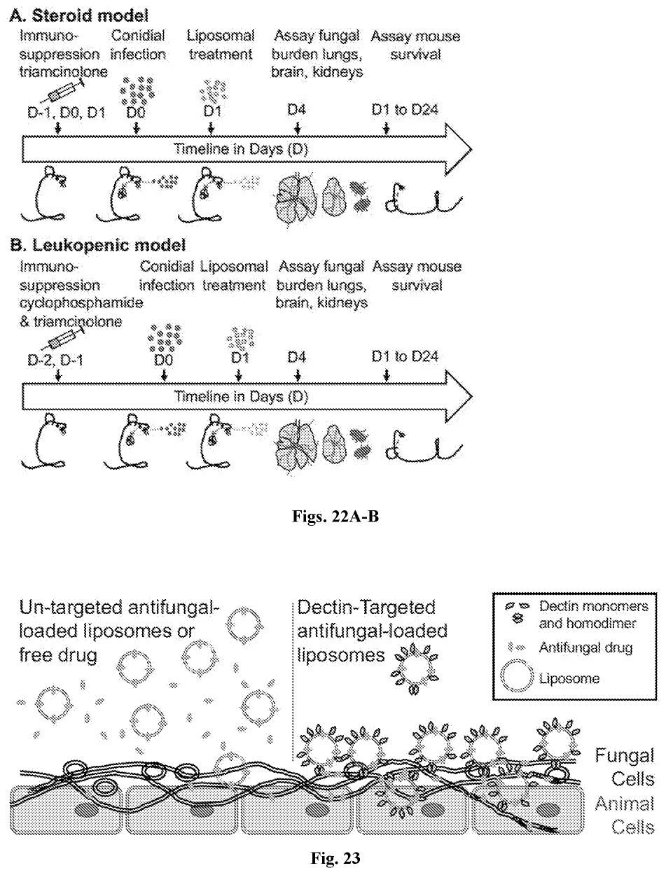

As described herein, the carbohydrate recognition domains of two C-type lectin receptors, Dectin-1 (DEC1) and Dectin-2 (DEC2), were used to target antifungal drug-loaded liposomes to fungal β-glucans and a-mannans, respectively. Dectin-targeted liposomes bind specifically to the cell walls and exopolysaccharide matrices of Aspergillus fumigatus, Candida albicans, and Cryptococcus neoformans. Untargeted liposomes do not bind specifically and can only passively deliver antifungals to fungal cells. As shown herein, DEC1- and DEC2-coated Amphotericin B (AmB)-loaded liposomes, DEC1-AmB-LLs and DEC2-AmB-LLs, bind efficiently to all stages developing A. fumigatus including un-swollen and swollen spores, germlings, and hyphae. They inhibit and/or kill A. fumigatus about 10-fold to about 90-fold more efficiently than AmBisome®-like liposomes AmB-LLs and reduce the in vitro effective doses of the drug by an order of magnitude. Whether this fungal cell targeting technology is effective in vivo in mouse models of aspergillosis was studied herein. The diagram in FIG. 23 (right side) illustrates Dectin-coated antifungal drug-loaded liposomes binding to fungal cells and their exopolysaccharide matrix, and hence, specifically targeting drug to the vicinity of fungal cells. This also concentrates antifungals away from the surface of mammalian cells. By contrast untargeted drugs passively deliver antifungals to all cells (FIG. 23, left side).

A. fumigatus is the principal causitive agent for aspergillosis, one of the four most life threatening fungal diseases. Globally there are estimated to be approximately 300,000 acute cases of aspergillosis. In 2017 alone, treatment costs for aspergillosis in the U.S. ranged from 58,000 to 105,000 US dollars per/patient with annual U.S. medical costs from Aspergillus infections totally 1.5 billion dollars. Aspergillosis accounts for 17% of the costs to treat all fungal infections. A. fumigatus is a common soil organism, but is also found in homes and work places. Most people breathe in thousands of spores every day without acquiring an infection. Patients at the greatest risk of developing life-threatening invasive fungal infections such as aspergillosis generally have weakened immune systems and/or have various lung diseases, increasing the chance of a fungal infection being established. Among immunocompromised patients, aspergillosis is the second most common fungal infection after candidiasis. Furthermore, the number of immunocompromised individuals, who are susceptible to various opportunistic fungal infections, is increasing due to the rise in cancer, stem cell, and organ transplant patients, who are on immunosuppressants.

Patients with aspergillosis are treated with antifungals such as Amphotericin B (AmB), caspofungin, and various azole drugs. Nearly all antifungal agents are hydrophobic and their low aqueous solubility presents a problem for drug delivery. Liposomal drug formulations with AmB intercalated into the bilipid membrane, such as AmBisome® or an equivalent such as AmB-LLs, penetrate more efficiently to various organ, penetrate the fungal cell wall and show reduced nephrotoxicity and less infusion toxicity at higher doses of AmB than detergent solubilized AmB (e.g., AmB-DOC). AmBisome® and their equivalent AmB-LLs are often used to kill A. fumigatus residing in biofilms. Dectin-targeting technology could improve antifungal activity in biofilms, because the Dectins bind to exopolysaccharide matrix that help compose biofilms. However, all antifungal agents, whether packaged in liposomes or not, have serious limitations due to a lack of sufficient fungicidal effect, increases in drug resistant fungal species, and host toxicity to cells and organs. For example, liposomal AmB causes renal toxicity (See, for example, Allen U. “Antifungal agents for the treatment of systemic fungal infections in children” Paediatrics & Child Health 15, 603-608 (2010); and Dupont B. “Overview of the lipid formulations of amphotericin B” J Antimicrob Chemother 49 Suppl 1, 31-36 (2002). The nickname “Amphoterrible” is frequently used by clinicians to describe amphotericin B, because of its side effects. Even with drug therapy, the case fatality rates for one year survival of aspergillosis patients generally ranges from 10% to only 90% depending upon the patient's underlying condition. For about 10% of patients invasive aspergillosis proceeds to cerebral aspergillosis, an infection of the brain, which has a mortality rate of 99%.

One of the goals for using the Dectin-based technology described herein was to demonstrate the increased efficacy of Dectin-2 coated liposomal drugs for the inhibition and killing of A. fumigatus in vivo in mouse models of immunosuppression-mediated aspergillosis (Desoubeaux et al., “Animal Models of Aspergillosis” Comp Med 68, 109-123 (2018)). Working near the minimum inhibitory concentration for AmB delivered by untargeted AmBisome®-like AmB-LLs, it was found that DEC2-AmB-LLs dramatically reduced the fungal burden in lungs, relative to AmB-LLs delivering the same AmB concentration.

Different publications suggest intranasal inoculum sizes ranging from 1×10⁴ to 1×10⁷ conidia of the A. fumigatus strain CEA10 are needed to induce lethal aspergillosis infection in immunosuppressed CD1 outbred Swiss mice (See Desoubeaux et al.; and Herbst et al. “A new and clinically relevant murine model of solid-organ transplant aspergillosis” Dis Model Mech 6, 643-651 (2013)). When delivered to mouse lungs, CEA10 appears to germinate faster and show more virulence than the more commonly used Af293 strain of A. fumigatus. The minimum inhibitory concentration (MIC) reported for AmB delivered as AmBisome® or AmBisome®-like AmB-LLs ranges widely from 0.06 to 1.0 mg/kg mouse weight in in vivo models and appears to vary widely depending upon the strain of A. fumigatus. By working at low doses of AmB for AmB-LLs we sought to demonstrate the maximum improved performance for Dectin-coated liposomes that are targeted to fungal cells in vivo relative to untargeted liposomes in vivo. Working near the in vitro MIC for AmB-LLs treatment of A. fumigatus growth and viability in culture that previously demonstrated one or two orders of magnitude improved performance of Dectin-targeted liposomes in vitro. Experiments described herein suggested that 0.2 mg AmB/kg delivered by AmBisome®-like AmB-LLs was sufficient to measure a slight drop in A. fumigatus levels in the lungs relative to infected control mice.

Heathy mice, like healthy humans, are naturally resistant to infection by Aspergillus spp. Hence, mice must be immunosuppressed to develop acute aspergillosis. Two different mouse models of immunosuppression, a steroid model and a leukopenic model, which differ in both in the quality of immune cell functions that are suppressed and in the severity of immunosuppression were used. Two protocols and time lines for examining the efficacy of Dectin-2 coated antifungal liposomes in a mouse model of pulmonary aspergillosis are shown in FIG. 22. Briefly, in the steroid model CD1 mice were immunosuppressed with the synthetic steroid triamcinolone or cortisone (FIG. 22A), while in the leukopenic model (FIG. 22B) mice were immunosuppressed with both an antimetabolite cyclophosphamide and a steroid. In the steroid- and leukopenc models of immunosuppression mediated aspergillosis, immunosuppressed mice received an oropharyngeal inoculation of 2×10⁶ and 100,000 to A. fumigatus conidia, strain CEA10 on Day 0 (D0), respectively. By D2 (Day 2 post infection) at least one large infection center of approximately 1 mm in diameter or larger was observed in nearly every lobe of the mouse lungs examined. These infection centers were composed of clusters of short hyphae (FIG. 24). On D1 post fungal inoculation (FIG. 22), infected mice were randomly separate into three groups. One treatment group received Dectin-2 coated DEC2-AmB-LLs delivering 0.2 mg AmB/kg mouse weight, a second liposome treatment group received untargeted AmB-LLs also delivering 0.2 mg AmB/kg, and the Control mock-treated group that received liposome dilution buffer only. All conidia and liposomes were administered to mice via an oropharyngeal delivery method as described previously for models of pulmonary diseases (See De Vooght et al. “Oropharyngeal aspiration: an alternative route for challenging in a mouse model of chemical-induced asthma,” Toxicology 259, 84-89 (2009)).

Fungal Burden Decreased Dramatically with DEC2-AmB-LL Treatment, Relative to Untargeted AmB-LLs.

The effect of antifungal liposome treatment on fungal burden in the lungs was examined. First, a steroid model of immunosuppression-mediated aspergillosis (FIG. 22A) was employed. CD1 Swiss mice were immunosuppressed with triamcinolone on D-1 and D3. On D0 they were infected with 2×10⁶ A. fumigatus conidia. Mice were treated on D1 with DEC2-AmB-LLs or with AmBisome®-like AmB-LLs liposomes delivering 0.2 mg AmB/kg mouse weight or with the Control buffer used to dilute the liposomes. Between D1 and D4 some of the animals in each treatment group had died, but at least three animals from each treatment group survived to D4. On D4 three randomly selected surviving animals from each group were euthanized and their lungs were harvested and weighed. Fungal burden was examined in the lungs by two methods (FIG. 25). Homogenized lung tissue was plated on rich growth media and 16 hr later the average number of Colony Forming Units (CFUs) per lung were estimated as shown in FIG. 25A. Mice treated with DEC2-AmB-LLs showed a 12.5-fold lower number of living fungal cells capable of forming colonies than AmB-LL treated mice and 20.5 fold lower than Control mice treated with liposome dilution buffer. Example images of the fields of fungal cell micro colonies used to generate these data are shown in FIG. 25B for AmB-LL treated mice and FIG. 25C for DEC2-AmB-LL treated mice. To have an independent estimate of the decrease in fungal burden generated by DEC2-AmB-LLs, quantitative polymerase chain reaction qPCR was used to measure the Relative Quantity of A. fumigatus ribosomal rDNA gene copies (FIG. 25D) on parallel samples of homogenized lung tissue from the same three mice in each of the three treatment groups. Based on an estimate of the Relative Quantity of rDNA, treatment with DEC2-AmB-LLs reduced fungal burden per lung 22-fold below the level for mice treated with AmBisome®-like AmB-LLs and 40-fold below the level in control mice. The concentration of AmB delivered in both fungal burden experiments, 0.2 mg/kg, is in the range of MICs reported for AmBisome® on A. fumigatus fungal burden in mouse models of aspergillosis. An approximately 40% drop in fungal burden was observed for AmB-LLs relative to control animals, suggesting concentration was at the high end of an MIC for the untargeted drug.

Second, a leukopenic mouse model of immunosuppression-mediated aspergillosis was employed. This model renders mice even more susceptible to aspergillosis and requires lower doses of conidia to establish an acute infection (FIG. 22B). CD1 Swiss mice were immunosuppressed with cyclophosphamide on D-3 and triamcinolone on D-1. On D0 they were infected with 5×10⁵ A. fumigatus conidia. Mice were treated on D1 with targeted DEC2-AmB-LLs or untargeted AmBisome®-like AmB-LLs liposomes delivering 0.2 mg AmB/kg mouse weight or with the control buffer used to dilute the liposomes. On Day 4 (D4) all the animals, but one, in all three treatment groups were still alive. One control buffer treated animal died in the morning of the 4th day and the remaining Control animals and one AmB-LL treated mouse showed reduced grooming. All the remaining animals in the AmB-LL and DEC2-AmB-LL treatment groups appeared relatively healthy. Three surviving animals from each treatment group were randomly selected, euthanized, and their lungs were harvested and fungal burden estimated by the two methods just described (FIG. 26). Mice treated with DEC2-AmB-LLs showed a 100-fold lower average number of Colony Forming Units (CFUs) per lung relative to AmB-LL treated mice (FIG. 26A). Clearly, targeting antifungal liposomes to fungal cells dramatically improved the efficacy of AmB packaged in liposomes. The average numbers of CFUs for the AmB-LL treated mice was lower, but was statistically indistinguishable from the control buffer treated animals. The high fungal burden in the AmB-LL treatment group was skewed by the fact that the one mouse that had showed reduced grooming had very high fungal titers. By contrast, one of the DEC2-AmB-LL treated mice had no detectable fungal colonies, contributing to the very low average CFUs for that treatment group. As an independent estimate of fungal burden, the Relative Quantity of A. fumigatus rDNA gene copies (FIG. 26B) on DNA prepared from parallel samples of homogenized lung tissue from the same mice was also examined. Treatment with DEC2-AmB-LLs reduced fungal burden per lung 600-fold below the level for mice treated with AmBisome®-like AmB-LLs. Plainly, using Dectin-2 to target liposomal AmB to fungal cells dramatically improves drug performance for reducing fungal burden in the lungs.

As described herein, an alternative to antibody targeting of liposomes was developed, by employing the C-type lectin carbohydrate recognition domain of Dectin-2 in a pan-fungal delivery system that targets liposomal drugs to mannans in the fungal cells and their exopolysaccharide matrices (FIG. 23). There was a concern that antibodies might not be as efficacious an approach for targeting liposomes to fungal pathogens as Dectin-coated liposomes, for two primary reasons. First, high affinity monoclonal antibodies may be too specific, reacting with only a particular subsets of glucan, mannan, xyloglucan crosslinks and mannoproteins, and hence, targeting only a small subset of fungal species or even a subset of fungal cells of the same species. The pan-antifungal activity of Dectin-targeted liposomes against diverse invasive fungal pathogens should make them more worthy of extensive and expensive clinical development. Second, less cumbersome methods were developed to produce the functional carbohydrate recognition domains of Dectin-1 and Dectin-2, DEC1 and DEC2, in E. coli at a few percent of the cost of producing the same molar concentrations of monoclonal antibodies. Again low reagent cost should encourage clinical development of a improved antifungal agents to treat acute invasive fungal diseases and superficial fungal infections.

The data presented herein demonstrate that DEC2-AmB-LLs have dramatically enhanced antifungal activity relative to untargeted AmBisome®-like AmB-LLs delivering the same low dose of AmB. Using two different models of immunosuppression-mediated mouse aspergillosis, DEC2-AmB-LLs reduced the fungal burden in the lungs by more by one to two orders of magnitude relative to an AmBisome® equivalent, AmB-LLs. It is expected that treatment with DEC2-AMB-LLs will provided improved rates of mouse survival in the mouse models described herein. Dramatic reductions in fungal burden should extrapolate into improved long term survival of patients.

Equally significant is the fact that a dramatic effect was shown using DEC2-AmB-LLs delivering only 0.2 mg AmB/kg mouse weight, concentrations near the low end of the MICs reported for liposomal AmB against A. fumigatus in vivo in mouse models of aspergillosis. In the studies described herein, using both models, AmB-LLs delivering 0.2 mg AmB/kg had a modest impact or no impact on fungal burden. Studies examining the efficacy of AmBisome® or AmB-LLs in pulmonary models of aspergillosis generally required concentrations of 5 to 20 mg AmB/kg to produce a maximum antifungal effect on fungal burden in the lungs and significantly improve mouse survival rates. Furthermore, multiple doses of AmB-LLs delivered over several days after infection were often necessary to insure a significant reduction in fungal burden and mouse survival. But herein we demonstrate that low doses of AmB delivered by targeted DEC2-AmB-LLs produced dramatic reductions in fungal burden, more dramatic than most reports of reduced fungal burden produced by AmBisome® in mouse models in the current literature. By significantly lowering the effective dose of AmB and the reducing the number of doses needed to control aspergillosis, Dectin-2-targeted liposomal AmB drugs, such as DEC2-AmB-LLs, should effectively control Aspergillus spp. infections, at concentrations that will not result in human organ toxicity.

Further, the data reported herein using Dectin-2 targeted liposomes delivering only 0.2 mg AmB/kg and obtaining orders of magnitude reductions in fungal burden, suggest that targeting antifungal loaded liposomes directly to fungal cells could be far more effective than targeting adjacent lung cells and tissues. It is likely that the close proximity of antifungal drug-loaded liposomes to fungal cells increases the local concentration of drug delivered to those cells. The in vitro data provided herein suggest that Dectin-2 primarily targets alpha-mannans in the exopolysaccharide matrix of A. fumigatus, and not the mannan content of its cell wall. We have not yet confirmed that Dectin-2 targets primarily the A. fumigatus exopolysaccharide matrix in vivo in mouse models.

There are a few different aspergillosis mouse models that mimic different types of immune suppression suffered by various patient populations, who are the most susceptible to aspergillosis. Herein, both steroid and leukopenic models of immunosuppression were used to mediate fungal infection (FIG. 22). In the steroid model, mice were pretreated with the synthetic glucocorticoid triamcinolone, which damages the adaptive immune response by suppressing some T cell and B cell activities, allowing increased proliferation of A. fumigatus hyphae into lung tissue. The steroid immunosuppression mouse model effectively mimics the immunodeficiency status of immunosuppressed patients receiving solid organ transplants and some patients receiving stem-cell transplants patients, who are particularly susceptible to aspergillosis (See Desoubeaux et al.). However, a disadvantage of this model is that there is excessive recruitment of neutrophils to affected tissue and particularly the lungs resulting in a dramatic inflammatory response. Because the immune system is only partially disabled, a relatively large fungal inoculum size was needed, at least 2×10⁶ conidia, to ensure that 100% of the mice developed acute aspergillosis. Most of the mice treated with smaller inoculum sizes ranging from 500,000 to 10⁶ conidia survived without antifungal treatment and at the other extreme nearly all those treated with 5×10⁶ all died by D3. The loss of some inoculated mice as early as D1 and D2 in the steroid model suggested to us that mice might be dying from inflammation and infection, before liposomal treatments had time to be effective. Nonetheless, DEC2-AmB-LLs targeted to fungal cells reduced fungal burden in the lungs relative to untargeted AmB-LLs delivering low doses of AmB.

In the next set of experiments, a leukopenic mouse model (FIG. 22B) was employed in which mice were pre-treated with cyclophosphamide and triamcinolone. Cyclophosphamide is a DNA synthesis inhibitor and an antimetabolite and induces apoptosis and rapid reduction in various leukocyte populations including B cells and memory T cells. This is added to the immunosuppressive effects of a steroid, triamcinolone. This model eliminates most of the innate and immune responses, and is far less restrictive of fungal cell proliferation than the steroid model. Severe leukopenia mimics the immunosuppressed status of hematopoietic stem cell transplant recipients. Because these mice will have severe leukopenia, smaller fungal inoculum sizes can be used, which slowed down the initial disease progression. It was hoped that slowing down the initial progression of infection would allow treatment with either AmB-LLs or DEC2-AmB-LLs to have the time to reduce fungal burden and in future experiments improve mouse survival.

Both models produced statistically convincing evidence that liposomes targeted to fungal cells reduced fungal burden in the lungs relative to untargeted liposomes delivering low doses of AmB. Such dramatic reductions in fungal burden in the lung at low doses of AmB as shown in FIG. 26 will undoubtedly extrapolate into improved rates of mouse survival and reduced animal toxicity.

As shown herein, DEC2-AmB-LLs bound efficiently to mannans in the exopolysaccharide matrices of in vitro grown A. fumigatus, C. albicans, and C. neoformans and improved the efficacy of killing all three fungal species. The experiments described above showed that Dectin-2 targeted liposome-mediated antifungal drug delivery dramatically improved the efficacy of inhibiting and killing A. fumigatus in vivo in two distinct mouse models of immunosuppression-mediated aspergillosis. Considering the in vitro data showing efficacious activity on multiple fungal pathogens and the in vivo mouse data presented herein on A. fumigatus, Dectin-2 targeting of antifungal drugs should have pan-antifungal applications against many other invasive fungal diseases including candidiasis and cryptococcosis and perhaps against dermatomycosis such as athlete's foot infections and onychomycosis of the nails caused by Trichophyton rubrum.

### Example 4

### Diagnostics

### Cell Culture

A. fumigatus strain CEA10 (ATCC MYA1163), was grown in Vogel's Minimal Media (VMM)+1% glucose⁷⁷ or RPMI 1640 media+1% glucose with no red indicator dye (ThermoFisher SKU— 11835-030, Waltham, Mass.) on 24 well polystyrene microtiter plates pre-coated with poly-L-lysine at 37° C. for 12 to 16 hr. Cells were washed with PBS (150 mM NaCl, 10 mM phosphate, pH 7.4.), fixed for 45 min with 4% formaldehyde and washed 3× with PBS before incubating with DEC2-BiFC reagent liposomes.

### Venus Fusion Proteins

The sequences of the codon-optimized E. coli expression constructs DEC2-VyN and DEC2-VC are shown in FIG. 27 along with their resulting protein sequences and some predicted protein properties. A 15 amino acid long GlySer rich flexible spacer separated the DEC2 sequence from the Venus fragment. Spacers as short as 2 amino acids are in common use in BiFC constructs such as pET-BiFC. It is understood that spacers of about 2, 3, 4, 5, 6, 7, 8, 9, 10, 11, 12, 13, 14, 15 amino acids or longer can be used in these constructs. In this example, a relatively long spacer was used to discourage spontaneous association of the complementary VyN and VC portions of the fusion proteins while placed in proximity on liposomes. Both gene sequences were synthesized and subcloned into the pET-45B expression vector by GenScript. The modified proteins were expressed in the BL21 strain of E. coli following 6 hr of IPTG induction at 37° C. Both were extracted from cells, purified on an Nickel affinity column and stored in denaturation buffer #1 (pH=8.0, 6 M GuHCl (Fisher BioReagents BP178), 0.1 M Na2HPO4/NaH2PO4, 10 mM Triethanolamine, 100 mM NaCl, 5 mM 2-mercaptoethanol, 0.1% Triton-X100) as described previously for mouse sDectin-1.

### DEC2-BiFC Reagent Construction

Samples of the two proteins at 5 μg/μL in this same GuHCl buffer with freshly added 5 mM 2-mercaptoethanol were adjusted to pH 8.3 with 1 M pH 10 triethanolamine and reacted with the reactive succinimidyl ester NHS moiety of a 4-molar excess of DSPE-PEG-3400-NHS (1,2-Distearoyl-sn-Glycero-3-Phosphoethanolamine (DSPE) conjugated polyethylene glycol (PEG), from Nanosoft polymers, 1544-3400) for 1 hr at 23° C. The PEG portion of the PEG-DSPE moiety makes these hydrophobic proteins slightly more soluble and DSPE is lipid that allows insertion into a liposomal membrane. Size exclusion chromatography through Bio-Gel P-6 acrylamide resin (Bio-Rad #150-0740) in renaturation and storage buffer RN #5 (0.1 M NaH2PO4, 10 mM Triethanolamine, pH 8.0, 1 M L-Arginine, 100 mM NaCl, 5 mM EDTA, 5 mM 2-mercaptoethanol) removed un-incorporated DSPE-PEG and GuHCl⁶⁹. The two modified proteins were stored at approximately 5 μg/μL in RN #5 until use in activity assays or incorporation into liposomes. SDS-PAGE was used to examine the level of protein purity after the GuHCl was removed by dialysis.

Remote-Loading of DEC2-VyN and DEC2-VC into Liposomes
Starting with sterile pegylated liposomes from FormuMax Sci. Inc.
(DSPC:CHOL:mPEG2000-DSPE, FormuMax #F10203A) small batches of liposomes were prepared by 30 min incubation at 60° C. in PBS by the addition of 0.5 moles percent each of DSPE-PEG-modified DEC2-VyN and DEC2-VC still in RN #5 buffer relative to 100% liposomal lipid to make DEC2-BiFC reagent (Table 2). Table 2 shows the chemical composition of DEC2-BiFC reagent liposomes discussed herein as moles DEC2-VyN and DEC2-VC, with the total amount of liposomal lipids representing 100 moles percent and compared to the amphotericin B loaded DEC2-AmB-LLs described recently. DEC2-BiFC reagent liposomes were diluted into liposome dilution buffer #2 (LDB2, 20 mM HEPES, 10 mM Triethoanolamine, 150 mM NaCl, 10 mM CaCl₂, 1 mM beta-mercaptoethanol (BME), 5% BSA pH 8.0) prior to use in fluorescence assays.

TABLE 2

| | DEC2-BiFC | |
|---|---|---|
| | reagent | DEC2-AmB-LLs |
| | liposomes | Ambati |
| Compound | this study | et al., 2019 |
| Additions to liposomes remotely | | |
| loaded for this study presented | | |
| as moles percent of total liposomal | | |
| lipids compared to previously | | |
| published DEC2-AmB-LLs | | |
| DEC2-PEG-DSPE | | 1.0 |
| DEC2-VyN-PEG-DSPE | 0.5 | 0.0 |
| DEC2-VC-PEG-DSPE | 0.5 | 0.0 |
| Amphotericin B | | 11.0 |
| Rhodamine Lissamine (DHPE- | 0.0 | 2.0 |
| Rhodamine) | | |
| Moles percent in addition to | 1.0 | 14.0 |
| total liposomal lipids | | |
| Lipids, lipidic vitamines, and | | |
| PEG-conjugated lipids defined | | |
| as 100% total liposomal lipids | | |
| mPEG2000-DSPE (N-Carbonyl- | 5.0 | 5.0 |
| methoxypolyethylene glycol 2000)- | | |
| distearoyl-glycerophosphoethanolamine) | | |
| DSPC (1,2-Distearol-sn-glycerol- | 50.0 | 50.0 |
| 3-phosphocholine) | | |
| CHOL (Chlosterol) | 45.0 | 45.0 |
| Total liposomal lipids = 100% | 100.0 | 100.0 |

### DEC2-BiFC Reagent Binding to Soluble Polysaccharides and Cells

Cell free assays. Polysaccharide stocks of soluble mannan from Saccharomyces cerevisiae (Sigma, Cat. #084K3789), laminarin from Laminaria digitate (Sigma-Aldrich, Cat. #L-9634), sucrose (Sigma), and Dextran-T40 (Pharmacosmos, Cat. #551000409007) purified from Lactobacillus spp. and size fractionated to 40,000 molecular weight were prepared at 10 mg/mL in PBS. These were then diluted 1:10 into LDB2 buffer containing DEC2-BiFC reagent liposomes before incubation at room temperature.

Cell based assays. Formalin fixed fungal cells were incubated with liposomes at 23° C. in liposome dilution buffer LDB2 (20 mM HEPES, 10 mM Triethoanolamine, 150 mM NaCl, 10 mM CaCl₂), 1 mM beta-mercaptoethanol (BME), 5% BSA pH 8.0), wherein the BME was added fresh. The DEC2-BiFC liposomes were diluted into LDB2 buffer before incubating with cells such that the concentration w/v of DEC2 protein component was between 2 μg/100 μL, 1.0 μg/100 μL or 0.5 μg/100 μL, respectively. Incubations were conducted at 23° C. or at 4° C. Images of fungal colonies stained with Venus green fluorescent DEC2-BiFC reagent liposomes were taken at 20× on an Olympus IX70 Inverted Microscope using GFP filters and parallel images were taken in bright field. Merge images were prepared in Adobe Photoshop by subtracting the green and blue channel data from the bright field image and adding back the green channel data from the green fluorescent channel.

### Results

Dectin-2 drifts as monomer in the membrane of lymphocytes but must form dimers for the extracellular C-type lectin receptor domain to bind alpha-mannans in fungal cell walls, exopolysaccharide matrices, biofilms, and polysaccharide fragments released into tissues and blood. Dimerization signals the immune system of a fungal infection. We employed Dectin-2's property of dimerization to engineer a cell-free pan-fungal detection system based on bimolecular fluorescence complementation (BiFC). The carbohydrate recognition domain of Dectin-2 (DEC2) was fused to the two complementary fragments of the green fluorescent protein VENUS, VyN and VC. DEC2-VyN. The DEC2-Venus fusion proteins were modified with a lipid carrier DSPE-PEG and floated together as monomers in a liposomal membrane to make DEC2-BiFC reagent. DEC2-BiFC reagent produced a fungal cell specific green fluorescent signal as it bound Aspergillus fumigatus. No fluorescent signal was produced that was not cell specific. The reagent produced a mannan-specific signal as it bound to soluble polysaccharides. Future efforts will explore the signal generated upon binding to Candida albicans, and Cryptococcus neoformans. DEC2-BiFC technology has potential as simple, rapid, one-step, diagnostic for life-threatening invasive fungal infections.

Invasive fungal infections are often misdiagnosed as non-fungal related diseases. A delay in antifungal drug therapy significantly increasing a patient's risk of mortality. Here are examples of the misdiagnosis of aspergillosis, candidiasis and cryptococcosis as non-fungal diseases. Many symptoms of aspergillosis and tuberculosis (TB) are the same, leading to an initial misdiagnosis and administration of anti-bacterial drugs to treat TB instead of treatment with antifungal drugs. Similarly, invasive candidiasis is often misdiagnosed as a bacterial infection and these patients are treated with broad spectrum antibacterial drugs. Furthermore, treatment of patients with antibacterial drugs encourages overgrowth by Candida spp. and increases a patient's chance of acquiring acute cases of invasive candidiasis. Cryptococcal meningitis is frequently misdiagnosed as brain cancer and patients are therefore not immediately treated with antifungal drugs. Provided herein is a reliable, rapid, sensitive pan-fungal diagnostic to identify fungal infections and distinguish them from non-fungal diseases with similar symptomology.

There are serious identifiable problems with the reliability, rapidity, and sensitivity of current methods of diagnosing fungal infections. The most common technique for diagnosing candidiasis is in vitro culture, which can detect as little as 1 cell per mL. And yet cell culture techniques and even PCR fail to detect candidiasis in 50% of patients with the disease, because shortly after infection Candida albicans yeast cells can hide out in host tissues evading fungal cell-based serum detection. Because Aspergillus and Candida species release fungal-specific polysaccharides into serum, sputum, bronchoalveolar lavage fluid, and urine, assays of fungal polysaccharides have been devised, in particular immunoassays of mannans and galactomannans and beta-glucans. However, current mannan and galactomannan assays for acute aspergillosis (e.g., Platelia Aspergillus, Bio-Rad) and beta-glucan pan-fungal assays (e.g., Fungitel, Beacon Diagnostics) are primarily multistep immunological ELISA sandwich assays. These commercial assays are not strictly reliable, either failing to detect infection or giving false positives for a significant fraction of patients. Furthermore, these assays are not easily adapted to point-of-care facilities, further delaying diagnosis. Cryptococcal meningitis is diagnosed using antibody based ELISA and lateral flow assays of Cryptococcal antigen and PCR assays for fungal DNA, but these assays have similar limitations, when applied to patient samples of cerebral spinal fluid. Again, early detection would save the lives of many patients with invasive fungal diseases. Clearly, more reliable, simple, rapid, one-step, point-of-care fungal diagnostics are desperately needed to reduce the mismanagement of patients with invasive fungal diseases.

Dectin-2 (CLEC6A gene) is C-type LECtin domain containing transmembrane receptor expressed on the surface of some classes of lymphocytes in mice and humans. Dectin-2 form dimers or multimers as it binds to fungal alpha-mannans and mannoproteins to signal the innate and adaptive immune system of fungal infection. Although affinity constants are hard to determine for the interaction of cell membrane receptors with high molecular weight polymorphic fungal polysaccharides. Estimates of the affinity of sDectin-2-protein fusions for model mannans taken from various publications suggest it has only a modest affinity for mannan containing polysaccharides. Yet, comparisons of the response of wild type to Clec4n^{-/-} null mice demonstrated Dectin-2 signaling responds maximally to 1 ng/mL concentrations of a mannan polymer, which implies affinity constants, Kds, in the sub-nanomolar range.

As shown herein, sDectin-2 (DEC2) coated liposomes bind very rapidly, efficiently, and relatively irreversibly to the exopolysaccharide matrices of Aspergillus fumigatus, Candida albicans, and Cryptococcus neoformans. Hence, development of a new pan-fungal diagnostic with DEC2 was undertaken. Candida, Cryptococcus and Aspergillus species belong to three evolutionarily disparate classes of fungi, the Saccharomycetes (phylum Ascomycota), Tremellomycetes (phylum Basidiomycota), and Eurotiomycetes (phylum Ascomycota), respectively. It is estimated that they separated from common ancestry relatively early in the evolution of the fungal kingdom, 0.8 to 1.3 billion years ago. Because DEC2 coated liposomes bound specifically to the extracellular matrix of all three, this suggests the mannans found in the extracellular matrix of most pathogenic fungi will be conserved enough in structure to be recognized by a DEC2-based diagnostic.

Herein, DEC2's property of dimer formation upon binding to mannan containing polysaccharides was used, to produce a fluorescent signal by Bimolecular Fluorescence Complementation (BiFC). DEC2 fused to the complementary fragments of the fluorescent protein VENUS were prepared and combined together in the same liposomes. The resulting DEC2-BiFC reagent liposomes produced a clear fluorescent signal when they bound to A. fumigatus cells. This technology could produce a simple and rapid one-step point-of-care assay for diverse pathogenic fungi, their exopolysaccharide matrices, biofilms, and released polysaccharides.

A model of a liposome-based one-step detection system using DEC2 is diagrammed in FIG. 28. It is based on DEC2's dimerization as it binds to fungal mannans and BiFC. Two separate sDectin-2 proteins were produced. The first was fused to the N-terminal portion of a green fluorescent protein Venus VyN (Venus residues 1-155, mutant T154M) to make DEC2-VyN (FIG. 28A, 28B). The VyN Venus mutant fragment has the advantage of reduced spontaneous association with the C-terminal Venus fragment, production of strong signal upon association with the C-terminal fragment, and few disadvantages relative to the N-terminal fragments of other fluorescent proteins or the unmodified VN fragment. The second protein produced was sDectin-2 fused to the C-terminal portion of Venus VC (Venus residues 155-238) to make DEC2-VC (FIG. 28A, 28B). This VC fragment has been partnered with VyN previously in successful BiFC applications. The exact sequences of the two encoding DNAs and two BiFC fusion proteins DEC2-VyN and DEC2-VC are shown in FIG. 27. Both encoding sequences were expressed in E. coli, the proteins extracted into a guanidine hydrochloride denaturing buffer, and purified by affinity chromatography using methods described herein. While still denatured, the complementary pairs of fusion proteins were each coupled via a free lysine residue to the amine reactive amphiphobic lipid reagent DSPE-PEG-NHS. The DSPE-PEG modified proteins were then inserted in equal molar ratios via their coupled DSPE moiety into the membrane of the same 100 nanometer diameter liposomes to make DEC2-BiFC reagent liposomes (FIG. 28A). The number of DEC2-fusion proteins inserted into each 100 nm liposome was limited to 1 mole percent DEC2 or 1,500 DEC2 molecules relative to the number of liposomal lipid molecules. In other words, there are approximately 750 DEC2-VyN and 750 DEC2-VC protein molecules in each liposome. The goal here was to use a concentration of the proteins that was low enough to avoid the spontaneous association of the two Venus fragments, which might generate a high background signal of fluorescence without any mannan binding, but to use a high enough concentration of DEC2 to promote effective binding, dimerization, and a strong signal when the reagent encountered fungal mannans. It was proposed that the DEC2-VyN and DEC2-VC monomers, floating together in a liposomal membrane, would form dimers or multimers almost exclusively as they bound fungal cell mannans. It was also proposed that DEC2 dimerization would promote the assembly of the complementary portions of Venus fluorescent protein −VyN and −VC to produce a BiFC signal.

A cell-free microtiter plate assay was preformed to demonstrate that the DEC2-BiFC reagent liposomes produced a fluorescent signal that was specific for the polysaccharide alpha-mannan. DEC2-BiFC reagent liposomes delivering 1 μg/100 μvenus L DEC2 protein to the media were incubated with soluble yeast mannan containing alpha-mannan, with laminarin a soluble beta-glucan, with sucrose a disaccharide of glucose and fructose, and with dextran an alpha-glucan. The signal was viewed in a fluorescent microtiter plate reader in the GFP channel. FIG. 29 shows that a statistically significantly higher levels of Venus green fluorescence were observed in the wells containing mannan (p=<0.01) relative to those with other polysaccharides. These results support the view that the signal is relatively mannan-specific as expected for Dectin-2. The maximum signal was observed after two hours and decreased with longer incubation.

A cell-based microscopic assay was preformed to demonstrate that the DEC2-BiFC reagent liposomes produced a fungal-cell specific fluorescent signal. A. fumigatus conidia were germinated on poly-L-lysine coated microtiter plates, grown overnight to the early hyphal stage forming small colonies of 300 to 500 microns in diameter. The cells were fixed in formalin and washed into liposome dilution buffer. The cells were incubated with DEC2-BiFC reagent delivering total DEC2 protein concentrations of 2 μg/100 μL, 1 μg/100 μL, and 0.5 μg/100 μL to the incubation media.

The BiFC signal was easily viewed in the GFP channel (Ex515/Em528) of an inverted fluorescent microscope. FIG. 30 shows the fluorescent signal formed, when DEC2-BiFC reagent (1 μg DEC2 protein/100 μL) bound to A. fumigatus colonies. The BiFC fluorescent signal is only observed in association with fungal cells, and hence, is fungal cell specific (FIG. 30B, D, F, H). Every one of over one hundred fungal cell colonies examined bound reagent. The signal was the strongest at the center of each fungal colony. Note, that the center of each colony has the thickest layer of cells and the oldest cells and has had the most time to produce an adhesive mannan-rich exopolysaccharide to aid in their adherence to the surface of the lysine coated plate. We've shown previously that DEC2 is particularly good at binding the expolysaccharide produced by A. fumigatus and exopolysaccharide deposition should enhance binding to the DEC2-BiFC reagent. There are long hyphae that stained over most of their length. The maximum signal was observed after two to three days of incubation. There were examples of fungal hyphae that did not show a fluorescent signal and presumably did not bind the reagent (White arrows, FIG. 30B). A similarly strong fungal cell-specific signal was also detected using liposomes delivering 2 μg DEC2/100 μL and a slightly weaker signal at 0.5 μg DEC2/100 μL. No fluorescence was observed in-between cells (FIG. 30B, D, F, H) or in control wells lacking fungal cells. Control cells incubated with buffer did not show a green fluorescent signal (FIGS. 30I & J).

### Venus as the Choice of Fluorescent Protein in the BiFC Construct

Venus was selected as the optimal fluorescent protein for constructing a BiFC pan-fungal diagnostic for two reasons. First, Venus was chosen, because out of several of the best studied yellow, green, and cerulean blue fluorescent proteins, the two fragments of Venus, the C-terminal fragment VC and the mutated N-terminal fragment VyN, had the lowest reported spontaneous rate of association, when not forced together by a partner protein. Spontaneous association produces a fluorescent signal independent of the fusion protein binding to the desired target and has been a problem with BiFC technology since its inception. A low spontaneous rate of association was considered essential to developing diagnostic reagent that did not produce false positives (i.e., fluorescent signal in the absence of fungal cell mannans). Second, Venus is one of the brightest fluorescent proteins and is derived from proteins in the very bright yellow fluorescent protein (YFP) family. Quantum yields (photons released per photon absorbed) are in the range of 0.5 to 0.6 (e.g., 50 to 60% of theoretical 100% yield)⁷⁰. This is similar to the quantum yield of rhodamine of 0.7, one of the most fluorescent low molecular weight fluorescent molecules in common use for fluorescent cytochemistry.

### BiFC Reagent Liposomes

DEC2-BiFC reagent liposomes containing both DEC2-VyN and DEC2-VC were designed instead of using the two soluble proteins DEC2-VyN and DEC2-VC free in solution for several reasons. First, DEC2 monomers are relatively insoluble and they are maintained in a soluble state by storing them in a 6 M guanidine hydrochloride buffer. Second, the addition of DSPE-PEG-moiety stabilizes and partially solubilized the DEC2 monomers and allows them to be stored for periods in mildly denaturing 1 M arginine containing buffers. This modification is essential for insertion of the modified proteins into liposomes. However, it is possible that this modified form of the two proteins could be used as a diagnostic reagent free of liposomes. Third, using liposomes the local concentration of DEC2 monomers in a stable reagent that was easier to manipulate was controlled. The goal here again was to achieve a high DEC2 concentration, but not one that was so high that it led to spontaneous association of the linked Venus fragments and a significant measurable fluorescent background. In these initial experiments, for example, it was found that 1,500 DEC2 monomers (i.e., 750 DEC2-VyN plus 750 DEC2-VC monomers) was effective. No spontaneous signal formation was observed even after the liposomes were stored for 2 months at 4° C. it is understood that the concentration of monomers can be increased or decreased. Fourth, and perhaps the most important, the property of avidity of the reagent liposomes should exponentially increase fluorescent signal strength. The potential for avidity is generated by having multiple mannan binding sites on each liposome such as observed for a pentameric IgM antibody. Once one DEC2-VyN and DEC2-VC pair bound to mannan in a mannan-rich fungal polysaccharide the signal should amplify as binding spreads to adjacent DEC2 molecules on the liposome binding to adjacent mannan moieties in a mannan rich polysaccharide. Avidity should also stabilize the signal, once it began to develop. A soluble BiFC diagnostic reagent cannot provide these advantages. The data presented herein were performed with the DEC2-BiFC reagent liposomes. The results are clearly positive, leaving little doubt that the fluorescent signal generated by the reagent is fungal-cell-specific.

In other examples, a shorter spacer can be used to force the VyN and VC fragments together more efficiently for more rapid signal formation. The systems described herein can be used to obtain a maximal signal within about 30 minutes, 40 minutes or 60 minutes of combining a DEC2-BiFC reagent with fungal samples.

The tight binding of Dectin-2's carbohydrate-recognition domain to alpha-mannans is concomitant with dimer formation in vivo on the surface of leukocytes. This property of dimer formation by the extracellular carbohydrate recognition domain DEC2 was successfully employed to develop a fungal-cell-specific diagnostic that works in vitro. DEC2 was fused to the two complementary fragments of the fluorescent protein VENUS to make DEC2-BiFC reagent liposomes. Simply by incubating the DEC2-BiFC reagent liposomes with A. fumigatus cells, they produced a clear fungal cell-specific green fluorescent signal. Hence, this technology can be used as a simple, one-step, point-of-care fungal diagnostic that will reduce the mismanagement of patients with invasive fungal diseases and perhaps a diagnostic for superficial fungal diseases.

Furthermore, there are dozens of C-type lectin binding proteins that form multimers as they bind diverse target fungal polysaccharides that could be employed to expand this diagnostic technology (See, for example, Hardison et al. “C-type lectin receptors orchestrate antifungal immunity,” Nat Immunol. 13, 817-822 (2012)).

1. A liposome comprising an antifungal agent and a targeting molecule that binds a target antigen on a fungal cell, wherein the targeting molecule is incorporated into the outer surface of the liposome and the antifungal agent is encapsulated in the liposome.

2. The liposome of claim 1, wherein the target antigen on the fungal cell is a fungal cell wall antigen or an antigen in an exopolysaccharide matrix associated with the fungal cell.

3. The liposome of claim 1, wherein the targeting molecule is a C-type lectin receptor, an antibody, a fungal cell wall binding protein, an exopolysaccharide binding protein, a chitin binding protein or a fragment thereof.

4. The liposome of claim 1, wherein the C-type lectin receptor is selected from the group consisting of Dectin-1, Dectin-2 and Dectin-3 or a fragment thereof.

5. (canceled)

6. The liposome of claim 1, wherein the antifungal agent is a polyene, azole or echinocandin antifungal agent.

7. (canceled)

8. The liposome of claim 1, wherein the targeting molecule or a fragment thereof is conjugated to a lipid or a pegylated lipid.

9. The liposome of claim 1, wherein the concentration of the antifungal drug is reduced as compared to the concentration of the antifungal drug encapsulated in a liposome that does not comprise a targeting molecule incorporated into the outer surface of the liposome.

10. The liposome of claim 1, wherein the liposome has decreased affinity for and/or is less toxic to an animal cell as compared to a liposome that does not comprise a targeting molecule incorporated into the outer surface of the liposome.

11. (canceled)

12. (canceled)

13. A plurality of liposomes according to claim 1.

14. A liposome comprising a targeting molecule that binds a target fungal cell antigen and a signal-generating molecule, wherein the targeting molecule is incorporated into the outer surface of the liposome and the signal-generating molecule generates a signal when the targeting molecule binds the target fungal cell antigen.

15. The liposome of claim 14, wherein the signal-generating molecule is linked to the targeting molecule.

16. The liposome of claim 14, wherein the signal-generating molecule is incorporated into or attached to the outer surface of the liposome.

17. (canceled)

18. (canceled)

19. A plurality of liposomes according to claim 14.

20. (canceled)

21. (canceled)

22. (canceled)

23. (canceled)

24. A pharmaceutical composition comprising the plurality of liposomes of claim 13.

25. A method of treating or preventing a fungal infection in a subject comprising administering to the subject having a fungal infection or at risk of developing a fungal infection an effective amount of the plurality of liposomes of claim 13.

26. (canceled)

27. The method of claim 25, wherein the fungal infection is an Aspergillus infection, a Cryptococcus infection, a Candida infection or a Trichophyton.

28. (canceled)

29. The method of claim 25, wherein the subject is immunocompromised.

30. The method of claim 25, wherein the subject has pneumonia, asthma, COPD, cystic fibrosis, tuberculosis, emphysema or sarcoidosis.

31. The method of claim 25, wherein the liposomes are administered topically, intranasally, systemically or via inhalation.

32. (canceled)

33. (canceled)

34. (canceled)

35. A method of making a plurality of liposomes comprising an antifungal agent and a targeting molecule that binds a target antigen on a fungal cell, wherein the targeting molecule is incorporated into the outer surface of each liposome and the antifungal agent is encapsulated in each liposome, the method comprising the steps of:
a) dissolving the antifungal agent in solvent for about 10 minutes to about 30 minutes, at about 60° C.;
b) encapsulating the antifungal agent into each liposome by mixing a plurality of liposomes in suspension with the antifungal/solvent solution of step a), for about 3 to about 5 hours, at about 60° C. or 37° C. for about 24-120 hours; and
c) incorporating the targeting molecule into the outer surface of each liposome by contacting the liposomes comprising the encapsulated antifungal agent with the targeting molecule, for about 45 minutes to about 90 minutes, at 60° C.

36. (canceled)

37. (canceled)

38. (canceled)

39. (canceled)

40. (canceled)

41. (canceled)

42. (canceled)

43. (canceled)

44. A method for detecting a fungal infection in a subject or a sample from a subject comprising:
a) contacting the subject or a sample from the subject with the plurality of liposomes of claim 14;
b) detecting a signal, wherein a signal indicates the presence of a fungal infection.

45.-49. (canceled)
